(19) United States
(12) Patent Application Publication
(10) Pub. No.: US 2004/0033973 A1
(43) Pub. Date: Feb. 19, 2004

(54) Compounds and oligomeric compounds comprising novel nucleobases

(76) Inventors: Muthiah Manoharan, Carlsbad, CA (US); Thazha P. Prakash, Carlsbad, CA (US); Kallanthottathil G. Rajeev, Solana Beach, CA (US)

Correspondence Address: WOODCOCK WASHBURN LLP, ONE LIBERTY PLACE - 46TH FLOOR, PHILADELPHIA, PA, 19103, US

(21) Appl. No.: 10/222,588

(22) Filed: Aug. 16, 2002

(51) Int. Cl. A61K 48/00; A61K 31/7072; A61K 31/7076; C07H 19/00; C07H 19/06

(52) U.S. Cl. 514/44; 514/45; 514/50; 536/27.2; 536/28.4

(57) ABSTRACT

The present invention relates to nucleoside compositions comprising novel nucleobases and oligomeric compounds comprising at least one such nucleoside. These oligomeric compounds typically have enhanced binding affinity properties compared to oligomeric compounds without the modification. The oligomeric compounds are useful, for example, for investigative and therapeutic purposes.

### FIELD OF THE INVENTION

The present invention relates to nucleoside compositions comprising novel nucleobases and oligomeric compounds comprising at least one such nucleoside. The oligomeric compounds of the present invention typically have enhanced binding affinity properties compared to oligomeric compounds without the modification. The oligomeric compounds are useful, for example, for investigative and therapeutic purposes.

### BACKGROUND OF THE INVENTION

Nearly all disease states in multicellular organisms involve the action of proteins. Classic therapeutic approaches have focused on the interaction of proteins with other molecules in efforts to moderate the proteins' disease-causing or disease-potentiating activities. In newer therapeutic approaches, modulation of the production of proteins has been sought. A general object of some current therapeutic approaches is to interfere with or otherwise modulate gene expression.

One method for inhibiting the expression of specific genes involves the use of oligonucleotides, particularly oligonucleotides that are complementary to a specific target messenger RNA (mRNA) sequence. Due to promising research results in recent years, oligonucleotides and oligonucleotide analogs are now accepted as therapeutic agents holding great promise for therapeutic and diagnostic methods.

Oligonucleotides and their analogs can be designed to have particular properties. A number of chemical modifications have been introduced into oligomeric compounds to increase their usefulness as therapeutic agents. Such modifications include those designed to increase binding affinity to a target strand, to increase cell penetration, to stabilize against nucleases and other enzymes that degrade or interfere with the structure or activity of the oligonucleotide, to provide a mode of disruption (terminating event) once the oligonucleotide is bound to a target, and to improve the pharmacokinetic properties of the oligonucleotide.

A variety of modified RNA nucleosides have been described in the art. Limbach, et al., Nucleic Acids Research, 1994, 22, 2183-96. It is know that such modifications can alter the properties of the nucloside. It has been observed, for example, that the modification of uridine (U) to 5,6-dihydrouridine (D) alters the nucleoside's sugar conformation. Agris, et al., Nucleic Acid Symposium Series, 1995, 33, 254-55.

A large number of nucleobase modifications, which were designed to enhance the binding affinity of antisense oligonucleotides to their complementary target strands, have been introduced (Beaucage, S. L.; Iyer, R. P. Tetrahedron 1993; 49, 6123-94; Cook, P. D. Annu. Rep. Med. Chem. 1998, 33, 313-325; Goodchild, J. Bioconjugate Chemistry, 1990; 1, 165-87; Uhlmann, E.; Peyman, A. Chem. Rev. 1990, 90, 543-84. For reviews see: Uhlmann, E.; Peyman, A. Chem. Rev. 1990, 90, 543-584; Milligan, J. F.; Matteucci, M. D.; Martin, J. C. J. Med. Chem. 1993, 36, 1923-37; Cook, P. D. Antisense Medicinal Chemistry; in Antisense Research and Application, A Handbook of Experimental Pharmacology (ed. Crooke, S. T.), pp. 51-101. Springer-Verlag, New York, 1998). Some heterocyclic modifications have been shown to enhance the binding affinity of nucleic acids through increased hydrogen bonding and/or base stacking interactions. Examples of such heterocyclic modifications include 2,6-diaminopurine, which allows for a third hydrogen bond with thymidine and replacement of the hydrogen atom at the C5 position of pyrimidine bases with a propynyl group, resulting in increased stacking interactions (Chollet, A.; Chollet-Damerius, A.; Kawashima, E. H. Chem. Scripta 1986, 26, 37-40; Wagner, R. W.; Matteucci, M. D.; Lewis, J. G.; Guttierrez, A. J.; Moulds, C.; Froehler, B. C. Science 1993, 260, 1510-1513).

More recently, several tricyclic cytosine analogs, such as phenoxazine, phenothiazine (Lin, K.-Y.; Jones, R. J.; Matteucci, M. J. Am. Chem. Soc. 1995, 117, 38733874) and tetrafluorophenoxazin (Wang, J.; Lin, K.-Y., Matteucci, M. Tetrahedron Lett. 1998, 39, 8385-8388), have been developed and have been shown to hybridize to guanine and, in case of tetrafluorophenoxazin, also with adenine. The tricyclic cytosine analogs have also been shown to enhance helical thermal stability by extended stacking interactions.

The helix-stabilizing properties of the tricyclic cytosine analogs are further improved with G-clamp, a cytosine analog with an aminoethoxy moiety attached to the rigid phenoxazine scaffold (Lin, K.-Y.; Matteucci, M. J. Am. Chem. Soc. 1998, 120, 8531-8532). Binding studies have demonstrated that a single G-clamp enhances the binding affinity of a model oligonucleotide to its complementary target DNA or RNA with a ΔTₘ of up to 18° relative to 5-methyl cytosine (dC5^{me}), the highest known affinity enhancement for a single modification. The gain in helical stability does not appear to compromise the binding specificity of the oligonucleotides, as the Tₘ data indicate an even greater discrimination between the perfectly matched and mismatched sequences as compared to dC5^{me}. The tethered amino group may serve as an additional hydrogen bond donor that interacts with the Hoogsteen face, namely the O6, of a complementary guanine. The increased affinity of G-clamp is thus most likely mediated by the combination of extended base stacking and additional hydrogen bonding.

The enhanced binding affinity of the phenoxazine derivatives together with their sequence specificity makes them potentially valuable nucleobase analogs for the development of more potent antisense-based drugs. Promising data have been derived from in vitro experiments demonstrating that heptanucleotides containing phenoxazine substitutions are capable of activating RNaseH, enhance cellular uptake, and exhibit an increased antisense activity (Lin, K.-Y.; Matteucci, M. J. Am. Chem. Soc. 1998, 120, 8531-8532). The activity enhancement was even more pronounced in the case of G-clamp, as a single substitution was shown to significantly improve the in vitro potency of a 20mer 2′-deoxyphosphorothioate oligonucleotide (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 35133518).

Despite these advances, a need exists in the art for the development of means to improve the binding affinity properties of oligomeric compounds.

### BRIEF SUMMARY OF THE INVENTION

The present invention concerns a compound of formula I:

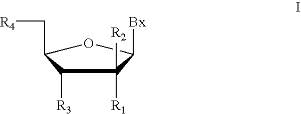

wherein:

R₁, R₂, and R₃ are selected such that:

R₃ is hydroxyl or protected hydroxyl, R₂ is H and R₁ is a sugar substituent group;

or R₃ is hydroxyl or protected hydroxyl, R₁ is H and R₂ is a sugar substituent group;

or R₂ is H, R₁ is hydroxyl or protected hydroxyl, and R₃ is a sugar substituent group;

R₄ is hydroxyl or protected hydroxy;

Bx has one of formulas II, III, IV, V, VI or VII:

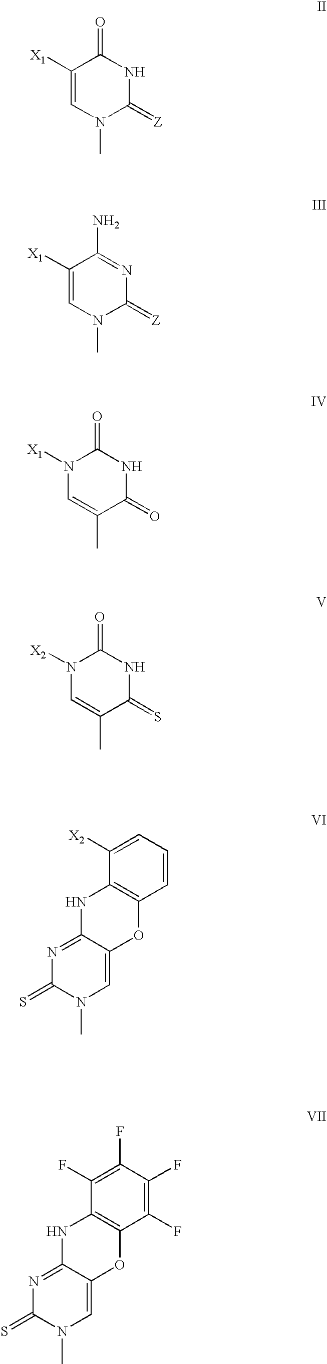

wherein:

X₁ is CH₂COOCH₃, CH₂COOCH₂CH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C≡CH, CH₂C(═Y)NRᵤRᵥ, or CH₂NRᵤRᵥ;

X₂ is H, CH₃, CH₂COOCH₃, CH₂COOCH₂CH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C≡CH, CH₂C(═Y)NRᵤRᵥ, or CH₂NRᵤRᵥ;

Y is S, O, or NH;

Z is S or O;

n is an integer from 1 to 10;

each Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached; and

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

In certain embodiments:

when Bx is formula II and Z is O then R₁ is not H, OH, OCH₃, OAc, protected hydroxyl or halogen; and

when Bx is formula II and Z is S then R₁ is not H, OH or protected hydroxyl;

when Bx is formula III, Z is O and X₁ is CH₂COOCH₃ then R₁ is not H;

when Bx is formula III, Z is O, X₁ is CH₂NH₂ then R₁ is not halogen; and

when Bx is formula III, Z is O and X₁ is CH₂C(═O)NRᵤRᵥ then at least one of Rᵤ and Rᵥ is not —(CH₂)₂NH₂.

In certain aspects of the invention, the sugar substituent group is C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)-Rₐ), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol or a polyether of the formula (—O-alkyl)ₘₐ;

wherein each Rₐ is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;

or said sugar substituent group has one of formula Iₐ or IIₐ:

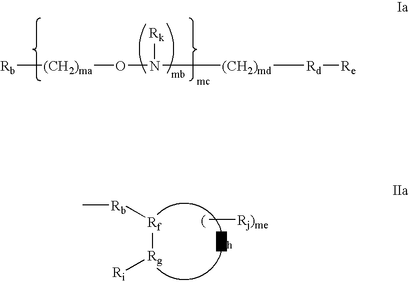

wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ;

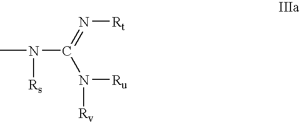

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or —RₓR_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rₗ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;

ma is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

In certain embodiments, the sugar substituent group is O(CH₂)₂OCH₃, O(CH₂)₂SCH₃, O(CH₂)₂ON(CH₃)₂, O(CH₂)₂O(CH₂)₂N(CH₃)₂, OCH₂C(═O)N(H)CH₃, OCH₃, O(CH₂)₂NH₂, O(CH₂)₂N(CH₃)₂, O(CH₂)₃NH₂, O(CH₂)₃N(H)CH₃, CH₂CH═CH₂, O(CH₂)₂S(O)CH₃, or fluoro.

In a further aspect, the invention concerns oligomeric compound comprising a plurality of nucleosides linked by internucleoside linking groups wherein at least one of said nucleosides is one of formulas VIII, IX or X:

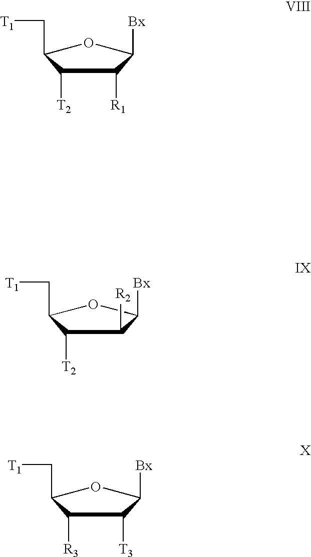

each T₁, T₂ and T₃ is, independently, hydroxyl, a protected hydroxyl or an internucleoside linking group covalently attaching a nucleoside, oligonucleoside, oligonucleotide or an oligomeric compound wherein at least one of T., T₂ and T₃ is an internucleoside linking group covalently attaching a nucleoside, oligonucleoside, oligonucleotide or an oligomeric compound;

each R₁, R₂ and R₃ is a sugar substituent group;

each Bx is one of formulas II, III, IV, V, VI or VII:

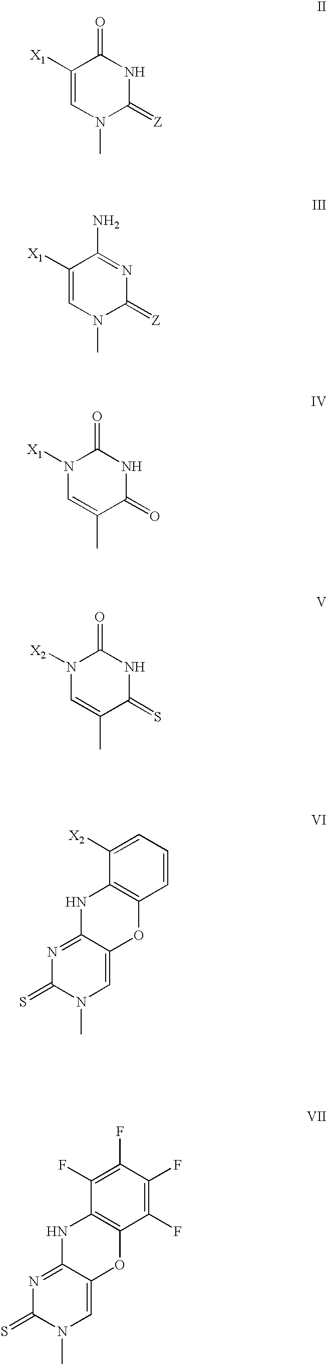

wherein:

X₁ is CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C≡CH, CH₂C(═Y)NRᵤRᵥ, or CH₂NRᵤRᵥ;

X₂ is H, CH₃, CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C≡CH, CH₂C(═Y)NRᵤRᵥ, or CH₂NRᵤRᵥ;

Y is S, O, or NH;

Z is S or O;

n is an integer from 1 to 10;

each Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached; and

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

In certain embodiments of the oligomeric compound:

when Bx is formula II and Z is O then R₁ is not H, OH, OCH₃, OAc, protected hydroxyl or halogen; and

when Bx is formula II and Z is S then R₁ is not H, OH or protected hydroxyl;

when Bx is formula III, Z is O and X₁ is CH₂COOCH₃ then R₁ is not H;

when Bx is formula III, Z is O, X₁ is CH₂NH₂ then R₁ is not halogen; and

when Bx is formula III, Z is O and X₁ is CH₂C(═O)NRᵤRᵥ then at least one of Rᵤ and Rᵥ is not —(CH₂)₂NH₂.

In certain embodiments, the oligomeric compounds are such that essentially each of the internucleoside linking groups contains a phosphorus atom. In some embodiments, the phosphorous containing internucleoside linking groups is, independently, selected from the group consisting of phosphodiester, phosphorothioate, chiral phosphorothioate, phosphorodithioate, phosphotriester, aminoalkylphosphotriester, methyl phosphonate, alkyl phosphonate, 5′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, selenophosphate and boranophosphate.

In other embodiments of the instant invention, the oligomeric compound is such that essentially each of the internucleoside linking groups is a non-phosphorus containing internucleoside linking group. In some embodiments, the non-phosphorus containing internucleoside linking groups is, independently, selected from the group consisting of morpholino, siloxane, sulfide, sulfoxide, sulfone, formacetyl, thioformacetyl, methylene formacetyl, thioformacetyl, sulfamate, methyleneimino, methylenehydrazino, sulfonate, sulfonamide, and amide. In certain embodiments, the non-phosphorus containing internucleoside linking groups is, independently, selected from the group consisting of CH₂—NH—O—CH₂—, —CH₂—N(CH₃)—O—CH₂—, —CH₂—O—N(CH₃)—CH₂—, —CH₂—N(CH₃)—N(CH₃)CH₂— and —O—N(CH₃)—CH₂—CH₂—.

In certain embodiments of the present invention, the oligomeric compound may comprise phosphorus and non-phosphorus containing internucleoside linking groups.

In further aspects of the present invention, the oligomeric compound comprises a gapmer, hemimer or inverted gapmer.

In a further aspect, the oligomeric compound comprises at least one of said monomeric subunits having a heterocyclic base moiety of formulas II, III, IV, V or VI comprises an arabinosy moiety. In a further embodiment, the oligomeric compound comprises a sugar substituent group selected from C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, -S-aralkyl, —N(H)-aralkyl, phthalimido (attached at N), halogen, amino, keto (—C(═O)—Rₐ), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol and a polyether of the formula (—O-alkyl)ₘₐ;

wherein each Rₐ is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;

or said sugar substituent group has one of formula Iₐ or IIₐ:

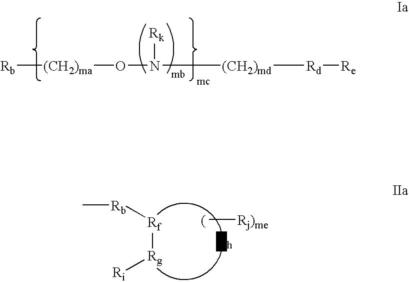

wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rr) or has formula IIIₐ;

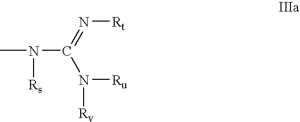

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or —Rₓ—R_{y};

Rₓ is a bond or a linking moiety;

R_{y} is a chemical functional group, a conjugate group or a solid support medium;

each Rₘ and Rₙ, is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;

ma is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

In another aspect, the oligomeric compound comprises one of formulas XII or XIII:

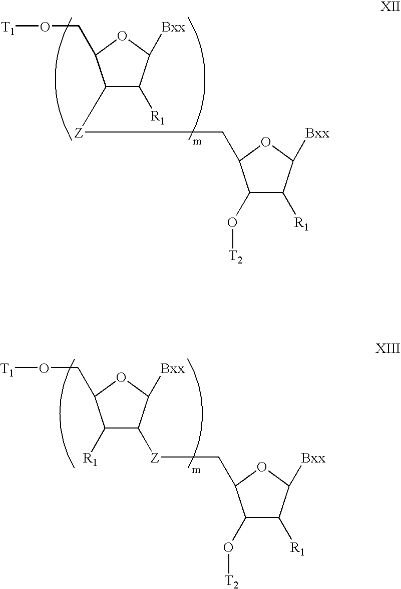

wherein:

each T₁ and T₂ is, independently, hydroxyl, a protected hydroxyl or an internucleoside linking group covalently attaching a nucleoside, oligonucleoside, oligonucleotide or an oligomeric compound;

each R₁ is a sugar substituent group;

m is from about 8 to about 80;

each Bxx is a heterocyclic base moiety with at least one heterocyclic base moiety having one of formulas II, III, IV, V, VI or VII:

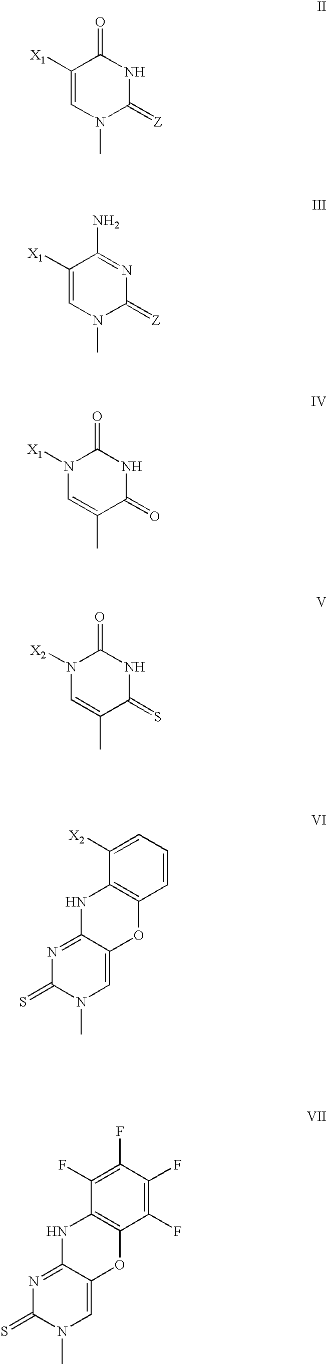

wherein:

X₁ is CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C═CH, CH₂C(═Y)NRᵤRᵥ, or CH₂NRᵤRᵥ;

X₂ is H, CH₃, CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C═CH, CH₂C(═Y)NRᵤRᵥ, or CH₂NRᵤRᵥ;

Y is S, O, or NH;

Z is S or O;

n is an integer from 1 to about 10;

each Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached; and

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

In certain embodiments:

when Bx is formula II and Z is O then R₁ is not H, OH, OCH₃, OAc, protected hydroxyl or halogen; and

when Bx is formula II and Z is S then R₁ is not H, OH or protected hydroxyl;

when Bx is formula III, Z is O and X₁ is CH₂COOCH₃ then R₁ is not H;

when Bx is formula III, Z is O, X₁ is CH₂NH₂ then R₁ is not halogen; and

when Bx is formula III, Z is O and X₁ is CH₂C(═O)NRᵤRᵥ then at least one of Rᵤ and Rᵥ is not —(CH₂)₂NH₂.

In some embodiments, the internucleoside linking group is a phosphorus-containing internucleoside linking group. In certain embodiments phosphorus-containing internucleoside linking group is selected from the group consisting of phosphorothioate, chiral phosphorothioate, phosphorodithioate, phosphotriester, aminoalkylphosphotriester, methyl phosphonate, alkyl phosphonate, 5′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, selenophosphate and boranophosphate.

In other embodiments, the internucleoside linking group is an amide, methylene (methylimino) or a formacetal.

In another aspect, the oligomer comprises heterocyclic base moieties in addition to said at least one having one of formulas II, III, IV, V, VI or VII are, independently, selected from the group consisting of adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

In certain embodiments of the oligomeric compound, m is from about 8 to about 50. In other emobidments, m is from about 12 to about 30.

In a further aspect, the invention concerns a pharmaceutical composition comprising at least one oligomeric compound described herein.

### DETAILED DESCRIPTION OF THE INVENTION

In the context of this invention, the term “oligomeric compound” refers to a polymeric structure capable of hybridizing a region of a nucleic acid molecule. This term includes oligonucleotides, oligonucleosides, oligonucleotide analogs, modified oligonucleotides and oligonucleotide mimetics. Oligomeric compounds can be prepared to be linear or circular and may include branching. They can be prepared single stranded or double stranded and may include overhangs. In general an oligomeric compound comprises a backbone of linked momeric subunits where each linked momeric subunit is directly or indirectly attached to a heterocyclic base moiety. The linkages joining the monomeric subunits, the monomeric subunits and the heterocyclic base moieties can be variable in structure giving rise to a plurality of motifs for the resulting oligomeric compounds including hemimers, gapmers and chimeras.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The terms “oligonucleotide analog” and “modified oligonucleotide” refers to oligonucleotides that have one or more non-naturally occurring portions which function in a similar manner to oligonulceotides. Such modified or substituted oligonucleotides are often preferred over native forms because of desirable properties such as, for example, enhanced cellular uptake, enhanced affinity for nucleic acid target and increased stability in the presence of nucleases.

In the context of this invention, the term “oligonucleoside” refers to nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts.

Representative United States patents that teach the preparation of the above-noted oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In the context of this invention, the term “oligonucleotide mimetic” refers to an oligonucleotide wherein the backbone of the nucleotide units has been replaced with novel groups. Although the term is intended to include oligomeric compounds wherein only the furanose ring or both the furanose ring and the internucleotide linkage are replaced with novel groups, replacement of only the furanose ring is also referred to in the art as being a sugar surrogate. Oligonucleotide mimetics can be further modified to incorporate one or more modified heterocyclic base moieties to enhance properties such as hybridization.

One oligonucleotide mimetic that has been reported to have excellent hybridization properties, is peptide nucleic acids (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative United States patents that teach the preparation of PNA compounds include, but are not limited to, U.S. Pat. No. 5,539,082; 5,714,331; and 5,719,262, each of which is herein incorporated by reference. Further teaching of PNA compounds can be found in Nielsen et al., Science, 1991, 254, 1497-1500.

PNA has been modified to incorporate numerous modifications since the basic PNA structure was first prepared. The basic structure is shown below:

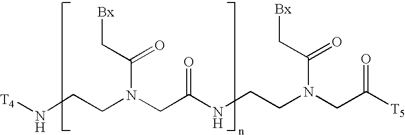

wherein

Bx is a heterocyclic base moiety;

T₄ is is hydrogen, an amino protecting group, —C(O)R₅, substituted or unsubstituted C₁-C₁₀ alkyl, substituted or unsubstituted C₂-C₁₀ alkenyl, substituted or unsubstituted C₂C₁₀ alkynyl, alkylsulfonyl, arylsulfonyl, a chemical functional group, a reporter group, a conjugate group, a D or L α-amino acid linked via the α-carboxyl group or optionally through the (ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₅ is —OH, —N(Z₁)Z₂, R₅, D or L α-amino acid linked via the α-amino group or optionally through the amino group when the amino acid is lysine or ornithine or a peptide derived from D, L or mixed D and L amino acids linked through an amino group, a chemical functional group, a reporter group or a conjugate group;

Z, is hydrogen, C₁-C₆ alkyl, or an amino protecting group;

Z₂ is hydrogen, C₁-C₆ alkyl, an amino protecting group, —C(═O)—(CH₂)ₙ-J-Z₃, a D or L α-amino acid linked via the ω-carboxyl group or optionally through the carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

each J is O, S or NH;

R₅ is a carbonyl protecting group; and

n is from 2 to about 50.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. A preferred class of linking groups have been selected to give a non-ionic oligomeric compound. The non-ionic morpholino-based oligomeric compounds are less likely to have undesired interactions with cellular proteins Morpholino-based oligomeric compounds are non-ionic mimics of oligonucleotides which are less likely to form undesired interactions with cellular proteins (Dwaine A. Braasch and David R. Corey, Biochemistry, 2002, 41(14), 4503-4510). Morpholino-based oligomeric compounds are disclosed in U.S. Pat. No. 5,034,506, issued Jul. 23, 1991. The morpholino class of oligomeric compounds have been prepared having a variety of different linking groups joining the monomeric subunits.

Morpholino nucleic acids have been prepared having a variety of different linking groups (L₂) joining the monomeric subunits. The basic formula is shown below:

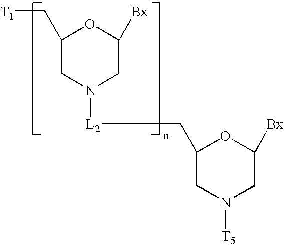

wherein

T₁ is hydroxyl or a protected hydroxyl;

T₅ is hydrogen or a phosphate or phosphate derivative;

L₂ is a linking group; and

n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. Coli RNase resulting in cleavage of the target RNA strand.

The general formula of CeNA is shown below:

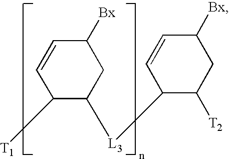

wherein

each Bx is a heterocyclic base moiety;

T₁ is hydroxyl or a protected hydroxyl; and

T₂ is hydroxyl or a protected hydroxyl.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

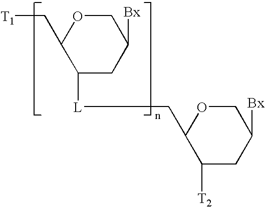

A further prefered modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage is preferably a methelyne (—CH2-)n group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. The basic structure of LNA showing the bicyclic ring system is shown below:

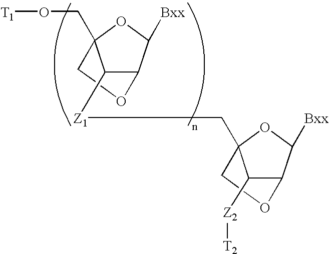

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 4453). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-modified oligonucleotides, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligonucleotides containing LNAs have been described (Wahlestedt et al., Proc. Natl. Acad. Sci. U.S. A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 36071-3630). LNAs and preparation thereof are also described in WO 98/39352 and WO 99/14226.

Analogs of INA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2‘-methylamino-LNA’s have been prepared and the thermal stability of their duplexes with complementary RNA and DNA strands has been previously reported.

Further oligonucleotide mimetics have been prepared to include bicyclic and tricyclic nucleoside analogs having the formulas (amidite monomers shown):

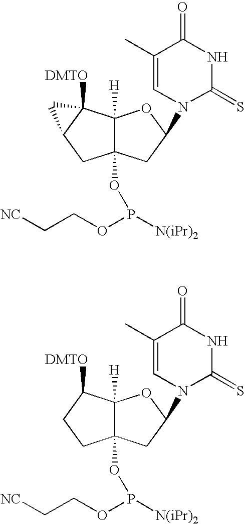

(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of Markush variables see: U.S. Pat. Nos. 5,874,553 and 6,127,346 herein incorporated by reference in their entirety) is shown below.

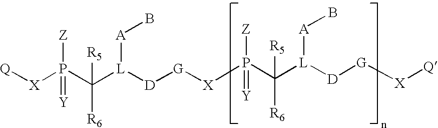

Another oligonucleotide mimetic has been reported wherein the furanosyl ring has been replaced by a cyclobutyl moiety.

The internucleotide linkage found in native nucleic acids is a phosphodiester linkage. This linkage has not been the linkage of choice for synthetic oligonucleotides that are for the most part targeted to a portion of a nucleic acid such as mRNA because of stability problems e.g. degradation by nucleases. Preferred internucleotide linkages and internucleoside linkages as is the case for non phosphate ester type linkages include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleoside linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In more preferred embodiments of the invention, oligomeric compounds have one or more phosphorothioate and/or heteroatom internucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester internucleotide linkage is represented as —O—P(═O)(OH)—O—CH₂—]. The MMI type internucleoside linkages are disclosed in the above referenced U.S. Pat. No. 5,489,677. Preferred amide internucleoside linkages are disclosed in the above referenced U.S. Pat. No. 5,602,240.

As used herein a sugar substituent group or nucleoside substituent group is a group that is covalently attached to the sugar portion of the nucleoside. Oligomeric compounds routinely incorporate modified nucleosides modified with nucleoside substituent groups to enhance one or more properties such as for example nuclease resistance or binding affinity. The 2′-position has been a preferred position for covalent attachment of nucleoside substituent groups. However, the 3′ and 5′-positions and the heterocyclic base moiety of selected nucleosides have also been modified with nucleoside substituent groups.

Representative substituent groups amenable to the present invention include C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, -S-aryl, —NH-aryl, —O-aralkyl, —S-aralkyl, —N(H)-aralkyl, phthalimido (attached at N), halogen, amino, keto (—C(═O)—Rₐ), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol or a polyether of the formula (—O-alkyl)ₘₐ. Each Rₐ is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group. Alkyl, alkenyl and alkynyl may be substituted or unsubstituted.

In some embodiments, the sugar nucleoside substituent group may be O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]2, where n and m are from 1 to about 10. Some embodiments may contain substituent groups such as heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466.786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative substituent groups include groups of formula Iₐ or IIₐ:

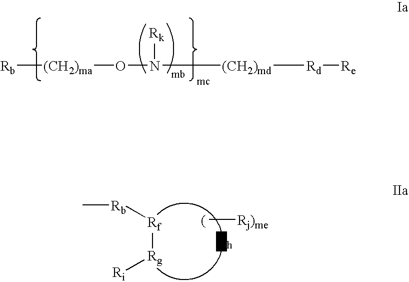

wherein:

R_{b} is O, S or NH;

R_{d} is a single bond, O or C(═O);

Rₑ is C₁-C₁₀ alkyl, N(Rₖ)(Rₘ), N(Rₖ)(Rₙ), N═C(Rₚ)(R_{q}), N═C(Rₚ)(Rr) or has formula IIIₐ;

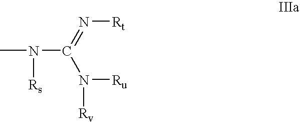

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyt, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;

Rₖ is hydrogen, a nitrogen protecting group or —Rₓ—R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;

ma is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Modified Oligonucleotides that are Conformationally Preorganized,” hereby incorporated by reference in its entirety.

Particularly preferred sugar substituent groups include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10.

Representative guanidino substituent groups are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 which is hereby incorporated by reference in its entirety.

Representative dimethylaminoethyloxyethyl substituent groups are disclosed in International Patent Application PCT/US99/17895, entitled “2′-O-Dimethylaminoethyloxyethyl-Modified Oligonucleotides”, filed Aug. 6, 1999, hereby incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain heterocyclic base moieties are particularly useful for increasing the binding affinity of the oligomeric compounds of the invention to complementary targets. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Id., pages 276-278) and are presently preferred base substitutions, even more particularly when combined with selected 2′-sugar modifications such as 2′-methoxyethyl groups.

Representative United States patents that teach the preparation of heterocyclic base moieties (modified nucleobases) include, but are not limited to, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, certain of which are commonly owned, and each of which is herein incorporated by reference, and commonly owned U.S. patent application Ser. No. 08/762,587, filed on Dec. 10, 1996, also herein incorporated by reference. Particularly useful bases are high affinity pyrimidine bases. These bases include those of formulas II, III, IV, V, VI and VII:

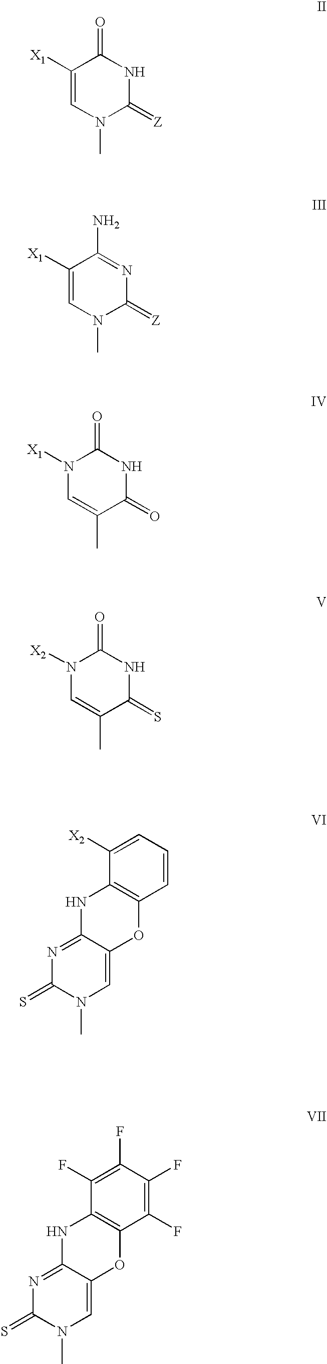

wherein:

X₁ is CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C≡CH, CH₂C(═Y)NRᵤRᵥ, or CH₂NRᵤRᵥ;

X₂ is H, CH₃, CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C—CH, CH₂C(═Y)NRᵤRᵥ, or CH₂NRᵤRᵥ;

Y is S, O, or NH;

Z is S or O;

n is an integer from 1 to 10;

each Rᵤ and Rᵥ is, independently, hydrogen, C(O)R_{w}, substituted or unsubstituted C₁-C₁₀ alkyl, substituted or unsubstituted C₂-C₁₀alkenyl; substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached; and

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl.

In certain embodiments of the invention:

when Bx is formula II and Z is O then R₁ is not H, OH, OCH₃, OAc, protected hydroxyl or halogen; and

when Bx is formula II and Z is S then R₁ is not H, OH or protected hydroxyl;

when Bx is formula m, Z is O and X₁ is CH₂COOCH₃ then R₁ is not H;

when Bx is formula III, Z is O, X₁ is CH₂NH₂ then R₁ is not halogen; and

when Bx is formula m, Z is O and X₁ is CH₂C(═O)NRᵤRᵥ then at least one of Rᵤ and Rᵥ is not —(CH₂)₂NH₂.

Other bases having polycyclic heterocyclic compounds in place of one or more heterocyclic base moieties may also be used in the present invention. A number of tricyclic heterocyclic comounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

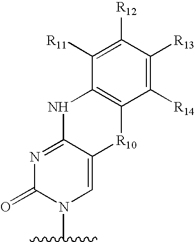

Representative cytosine analogs that make three hydrogen bonds with a guanosine in a second strand include 1,3-diazaphenoxazine-2-one (R₁₀₌O, R₁₁—R₁₄═H) [Kurchavov, et al., Nucleosides and Nucleotides, 1997, 16, 1837-1846], 1,3-diazaphenothiazine-2-one (R₁₀=S, R₁₁—R₁₄═H), [Lin, K.-Y.; Jones, R. J.; Matteucci, M. J. Am. Chem. Soc. 1995, 117, 3873-3874] and 6,7,8,9-tetrafluoro-1,3-diazaphenoxazine-2-one (R₁₀=O, R₁₁—R₁₄═F) [Wang, J.; Lin, K.-Y., Matteucci, M. Tetrahedron Lett. 1998, 39, 8385-8388]. Incorporated into oligonucleotides, these base modifications were shown to hybridize with complementary guanine and the latter was also shown to hybridize with adenine and to enhance helical thermal stability by extended stacking interactions.

Further helix-stabilizing properties have been observed when a cytosine analog/substitute has an aminoethoxy moiety attached to the rigid 1,3-diazaphenoxazine-2-one scaffold (R₁₀═O, R₁₁=—O—(CH₂)₂—NH₂, R₁₂₋₁₄=H) [Lin, K.-Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety. Such compounds include those having the formula:

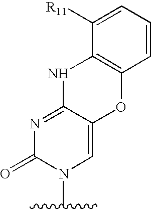

wherein R₁₁ includes (CH₃)₂N—(CH₂)₂—O—; H₂N—(CH₂)₃—; Ph-CH₂—O—C(═O)—N(H)—(CH₂)₃—; H₂N—; Fluorenyl-CH₂—O—C(═O)—N(H)—(CH₂)₃—; Phthalimidyl-CH₂—O—C(═O)—N(H)—(CH₂)₃—; Ph-CH₂—O—C(═O)—N(H)—(CH₂)₂—O—; Ph-CH₂—O—C(═O)—N(H)—(CH₂)₃—O—; (CH₃)₂N—N(H)—(CH₂)₂—O—; Fluorenyl-CH₂—O—C(═O)—N(H)—(CH₂)₂—O—; Fluorenyl-CH₂—O—C(═O)—N(H)—(CH₂)₃—O—; H₂N—(CH₂)₂—O—CH₂—; N₃—(CH₂)₂—O—CH₂—; H₂N—(CH₂)₂—O—, and NH₂C(═NH)NH—.

Also disclosed are tricyclic heterocyclic compounds of the formula:

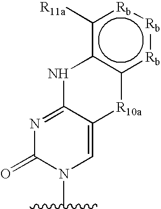

wherein

R₁₀ₐ is O, S or N—CH₃;

R₁₁ₐ is A(Z)ₓ₁, wherein A is a spacer and Z independently is a label bonding group bonding group optionally bonded to a detectable label, but R₁₁ₐ is not amine, protected amine, nitro or cyano;

X₁ is 1, 2 or 3; and

R_{b} is independently —CH═, —N═, —C(C₁₋₈ alkyl)=or —C(halogen)=, but no adjacent R_{b} are both —N═, or two adjacent R_{b} are taken together to form a ring having the structure:

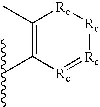

where R_{c} is independently —CH═, —N═, —C(C₁₋₈ alkyl)=or —C(halogen)=, but no adjacent R_{b} are both —N═.

The enhanced binding affinity of the phenoxazine derivatives together with their sequence specificity makes them valuable nucleobase analogs for the development of antisense-based drugs. In fact, promising data have been derived from in vitro experiments demonstrating that heptanucleotides containing phenoxazine substitutions are able to activate RNaseH, enhance cellular uptake and exhibit an increased antisense activity [Lin, K.-Y.; Matteucci, M. J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518].

Further tricyclic and tetracyclic heteroaryl compounds amenable to the present invention include those having the formulas:

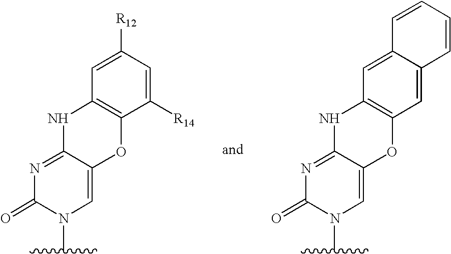

wherein R₁₄ is NO₂ or both R₁₄ and R₁₂ are independently —CH₃. The synthesis of these compounds is dicslosed in U.S. Pat. No. 5,434,257, which issued on Jul. 18, 1995, U.S. Pat. No. 5,502,177, which issued on Mar. 26, 1996, and U.S. Pat. No. 5,646, 269, which issued on Jul. 8, 1997, the contents of which are commonly assigned with this application and are incorporated herein in their entirety.

Further polycyclic heterocyclic base moieties having the formula:

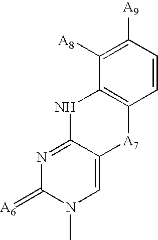

wherein:

A₆ is O or S;

A₇ is CH₂, N—CH₃, O or S;

each A₈ and A₉ is hydrogen or one of A₈ and A₉ is hydrogen and the other of A₈ and A₉ is selected from the group consisting of:

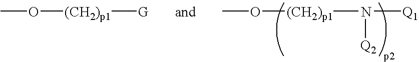

wherein:

G is —CN, —OA₁₀, —SA₁₀, —N(H)A₁₀, —ON(H)A₁₀ or —C(═NH)N(H)A₁₀;

Qᵢ is H, —NHA₁₀, —C(═O)N(H)A₁₀, —C(═S)N(H)A₁₀ or —C(═NH)N(H)A₁₀;

each Q₂ is, independently, H or Pg;

A₁₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids;

Pg is a nitrogen, oxygen or thiol protecting group;

each p1 is, independently, from 2 to about 6;

p2 is from 1 to about 3; and

p3 is from 1 to about 4;

are disclosed in Unites States patent application Ser. No. 09/996,292 filed Nov. 28, 2001, which is commonly owned with the instant application, and is herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in United States patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, modified oligonucleotides, oligonucleotide analogs, oligonucleosides and/or oligonucleotide mimetics as described above. Such compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

The compounds described herein may have asymmetric centers. Unless otherwise indicated, all chiral, diastereomeric, and racemic forms are included in the present invention. Geometric isomers may also be present in the compounds described herein, and all such stable isomers are contemplated by the present invention. It will be appreciated that compounds in accordance with the present invention that contain asymmetrically substituted carbon atoms may be isolated in optically active or racemic forms or by synthesis.

The present invention includes all isotopes of atoms occurring in the intermediates or final compounds. Isotopes include those atoms having the same atomic number but different mass numbers. By way of example, and without limitation, isotopes of hydrogen include tritium and deuterium.

In preferred embodiments of the invention, oligomeric compounds are synthesized on support media derivatized with a thioester group according to standard oligonucleotide synthesis procedures. In some embodiments of the invention, the support-bound oligonucleotides are then treated with spermine plus thiophenol in aqueous MeCN to yield polyethyleneamine-conjugated oligomeric compounds. In other embodiments of the invention, the support-bound oligonucleotides are treated with polyethyleneimines and thiophenol, the reaction mixture is diluted with concentrated aqueous ammonium hydroxide, and the solution is heated and evaporated. The residue is dissolved in water and neutralized with aqueous AcOH, resulting in precipitation of oligonucltotide conjugates complexed with excess polyethyleneimine. The precipitate is washed with MeCN and ether and re-dissolved in a mixture of piperidine and DMSO. The polyethyleneamine-conjugated oligomeric compounds are then purified on a Sephadex G25 column, and then further purified by reverse-phase HPLC.

Standard procedures for the synthesis of oligomeric compounds involve attachment of a first nucleoside or larger nucleosidic synthon to support media followed by iterative elongation of the nucleoside or nucleosidic synthon to yield a final oligomeric compound. In some embodiments of the invention, oligomeric compounds are synthesized by attaching a 5′-O-protected nucleoside to a solid support derivatized with a thioester group, deprotecting the 5′-hydroxyl of the nucleoside with a deprotecting reagent, reacting the deprotected 5′-hydroxyl with a 5′-protected activated phosphorus compound to produce a covalent linkage therebetween, oxidizing or sulfurizing the covalent linkage, and repeating the deprotecting, reacting, and oxidizing steps to produce an oligomer attached to the derivatized support media.

Support media can be selected to be insoluble or to have variable solubility in different solvents, which allows the growing oligomer to be kept out of or in solution as desired. Traditional solid supports are insoluble, while soluble supports have recently been introduced. Soluble polymer supports allow the bound oligomer to be precipitated or dissolved at desired points in the synthesis (Gravert et al., Chem. Rev., 1997, 97, 489-510).

Representative support media amenable to the present invention include, without limitation, controlled pore glass (CPG); oxalyl-controlled pore glass (see, e.g., Alul, et al., Nucleic Acids Research 1991, 19, 1527); TENTAGEL Support, (see, e.g., Wright, et al., Tetrahedron Letters 1993, 34, 3373); and POROS, a copolymer of polystyrene/divinylbenzene available from Perceptive Biosystems. Use of poly(ethylene glycol) of molecular weight between 5 and 20 kDa as a soluble support media for large-scale synthesis of phosphorothioate oligonucleotides is described in Bonora et al., Organic Process Research & Development, 2000, 4, 225-231. Equipment for such synthesis is sold by several vendors including, for example, Applied Biosystems (Foster City, Calif.).

Other means for synthesis of oligomeric compounds may additionally or alternatively be employed. Techniques for synthesizing oligonucleotides, such as phosphorothioates and alkylated derivatives, are familiar to those of ordinary skill in the art.

Activated phosphorus compositions (e.g. compounds having activated phosphorus-containing substituent groups) may be used in coupling reactions for the synthesis of oligomeric compounds. As used herein, the term “activated phosphorus composition” includes monomers and oligomers that have an activated phosphorus-containing substituent group that reacts with a hydroxyl group of another monomeric or oligomeric compound to form a phosphorus-containing internucleotide linkage. Such activated phosphorus groups contain activated phosphorus atoms in p^{III} valence state. Such activated phosphorus atoms are known in the art and include, but are not limited to, phosphoramidite, H-phosphonate, phosphate triesters and chiral auxiliaries. A preferred synthetic solid phase synthesis utilizes phosphoramidites as activated phosphates. The phosphoramidites utilize p^{III} chemistry. The intermediate phosphite compounds are subsequently oxidized to the p^{V} state using known methods to yield, in a preferred embodiment, phosphodiester or phosphorothioate internucleotide linkages. Additional activated phosphates and phosphites are disclosed in Tetrahedron Report Number 309 (Beaucage and Iyer, Tetrahedron, 1992, 48, 2223-2311).

A representative list of activated phosphorus-containing monomers or oligomers include those having the formula:

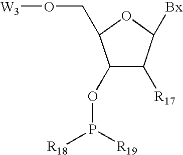

wherein

each Bx is, independently, a heterocyclic base moiety or a blocked heterocyclic base moiety; and

each R₁₇ is, independently, H, a blocked hydroxyl group, a sugar substituent group, or a blocked substituent group;

W₃ is an hydroxyl protecting group, a nucleoside, a nucleotide, an oligonucleoside or an oligonucleotide;

R₁₈ is N(L₁)L₂,

each L₁ and L₂ is, independently, C₁₋₆ alkyl;

or L₁ and L₂ are joined together to form a 4- to 7-membered heterocyclic ring system including the nitrogen atom to which L₁ and L₂ are attached, wherein said ring system optionally includes at least one additional heteroatom selected from O, N and S; and

R₁₉ is X₁;

X₁ is Pg-O—, Pg-S—, C₁-C₁₀ straight or branched chain alkyl, CH₃(CH₂)ₚ₅—O— or R₂₀R₂₁N—;

p5 is from 0 to 10;

Pg is a protecting group;

each R₂₀ and R₂₁ is, independently, hydrogen, C₁-C₁₀ alkyl, cycloalkyl or aryl;

or optionally, R₂₀ and R₂₁, together with the nitrogen atom to which they are attached form a cyclic moiety that may include an additional heteroatom selected from O, S and N; or

R₁₈ and R₁₉ together with the phosphorus atom to which R₁₈ and R₁₉ are attached form a chiral auxiliary.

Groups attached to the phosphorus atom of internucleotide linkages before and after oxidation (R₁₈ and R₁₉) can include nitrogen containing cyclic moieties such as morpholine. Such oxidized internucleoside linkages include a phosphoromorpholidothioate linkage (Wilk et al., Nucleosides and nucleotides, 1991, 10, 319-322). Further cyclic moieties amenable to the present invention include mono-, bi- or tricyclic ring moieties that may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkylsulfonyl, nitro, sulfide, sulfone, sulfonamide, thiol and thioalkoxy. A preferred bicyclic ring structure that includes nitrogen is phthalimido.

In the context of this specification, alkyl (generally C₁-C₂₀), alkenyl (generally C₂-C₂₀), and alkynyl (generally C₂-C₂₀) groups include, but are not limited to, substituted and unsubstituted straight chain, branch chain, and alicyclic hydrocarbons, including methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and other higher carbon alkyl groups. Further examples include 2-methylpropyl, 2-methyl-4-ethylbutyl, 2,4-diethylbutyl, 3-propylbutyl, 2,8-dibutyldecyl, 6,6-dimethyloctyl, 6-propyl-6-butyloctyl, 2-methylbutyl, 2-methylpentyl, 3-methylpentyl, 2-ethylhexyl and other branched chain groups, allyl, crotyl, propargyl, 2-pentenyl and other unsaturated groups containing a pi bond, cyclohexane, cyclopentane, adamantane as well as other alicyclic groups, 3-penten-2-one, 3-methyl-2-butanol, 2-cyanooctyl, 3-methoxy-4-heptanal, 3-nitrobutyl, 4-isopropoxydodecyl, 4-azido-2-nitrodecyl, 5-mercaptononyl, 4-amino-1-pentenyl as well as other substituted groups. Representative alkyl substituents are disclosed in U.S. Pat. No. 5,212,295, at column 12, lines 41-50, hereby incorporated by reference in its entirety.

A number of chemical functional groups can be introduced into compounds of the invention in a blocked form and subsequently deblocked to form a final, desired compound. Such groups can be directly or indirectly attached at the heterocyclic bases, the internucleoside linkages and the sugar substituent groups at one or more of the 2′, 3′ and 5′-positions. Protecting groups can be selected to block functional groups located in a growing oligomeric compound during iterative oligonucleotide synthesis while other positions can be selectively deblocked as needed. In general, a blocking group renders a chemical functionality of a larger molecule inert to specific reaction conditions and can later be removed from such functionality without substantially damaging the remainder of the molecule (Greene and Wuts, Protective Groups in Organic Synthesis, 3rd ed, John Wiley & Sons, New York, 1999). For example, the nitrogen atom of amino groups can be blocked as phthalimido groups, as 9-fluorenylmethoxycarbonyl (FMOC) groups, and with triphenylmethylsulfenyl, t-BOC or benzyl groups. Carboxyl groups can be blocked as acetyl groups. Representative hydroxyl protecting groups are described by Beaucage et al., Tetrahedron 1992, 48, 2223. Preferred hydroxyl protecting groups are acid-labile, such as the trityl, monomethoxytrityl, dimethoxytrityl, trimethoxytrityl, 9-phenylxanthine-9-yl (Pixyl) and 9-(p-methoxyphenyl)xanthine-9-yl (MOX).

Chemical functional groups can also be “blocked” by including them in a precursor form. Thus, an azido group can be considered a “blocked” form of an amine since the azido group is easily converted to the amine. Further representative protecting groups utilized in oligonucleotide synthesis are discussed in Agrawal, et al., Protocols for Oligonucleotide Conjugates, Eds, Humana Press; New Jersey, 1994; Vol. 26 pp. 1-72.

Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p=-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Examples of thiol (sulfur) protecting groups include, but are not limited to, benzyl, substituted benzyls, diphenylmethly, phenyl, t-butyl, methoxymethyl, thiazolidines, acetyl and benzoyl. Further thiol protecting groups are illustrated in Greene and Wuts, ibid.

Additional amino-protecting groups include but are not limited to, carbamate-protecting groups, such as 2-trimethylsilylethoxycarbonyl (Teoc), 1-methyl-1-(4-biphenylyl)ethoxycarbonyl (Bpoc); t-butoxycarbonyl (BOC), allyloxycarbonyl (Alloc), 9-fluorenylmethyloxycarbonyl (Fmoc), and benzyloxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl. Equivalents of these amino-protecting groups are also encompassed by the compounds and methods of the present invention.

Some preferred amino-protecting groups are stable to acid treatment and can be selectively removed with base treatment, which makes reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p.1), and various substituted sulfonylethyl carbamates exemplified by the Nsc group (Samukov et al., Tetrahedron Lett, 1994, 35:7821; Verhart and Tesser, Rec. Trav. Chim. Pays-Bas, 1987, 107:621).

In some especially preferred embodiments, the nucleoside components of the oligomeric compounds are connected to each other by optionally protected phosphorothioate internucleoside linkages. Representative protecting groups for phosphorus containing internucleoside linkages such as phosphite, phosphodiester and phosphorothioate linages include β-cyanoethyl, diphenylsilylethyl, δ-cyanobutenyl, cyano p-xylyl (CPX), N-methyl-N-trifluoroacetyl ethyl (META), acetoxy phenoxy ethyl (APE) and butene-4-yl groups. See for example U.S. Pat. Nos. 4,725,677 and Re. 34,069 (β-cyanoethyl); Beaucage, S. L. and Iyer, R. P., Tetrahedron, 49 No. 10, pp. 1925-1963 (1993); Beaucage, S. L. and Iyer, R. P., Tetrahedron, 49 No. 46, pp. 10441-10488 (1993); Beaucage, S. L. and Iyer, R. P., Tetrahedron, 48 No. 12, pp. 2223-2311.

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which-is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of a particular target gene is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds can be prepared to hybridize to nucleic acids encoding a particular protein, enabling sandwich and other assays to easily be constructed to exploit this fact. Hybridization of the antisense oligonucleotides of the invention with a nucleic acid encoding a particular protein can be detected by means known in the art. Such means may include conjugation of an enzyme to the oligonucleotide, radiolabelling of the oligonucleotide or any other suitable detection means. Kits using such detection means for detecting protein levels in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. No. 08/886,829 (filed Jul. 1, 1997), Ser. No. 09/108,673 (filed Jul. 1, 1998), Ser. No. 09/256,515 (filed Feb. 23, 1999), Ser. No. 09/082,624 (filed May 21, 1998) and Ser. No. 09/315,298 (filed May 20, 1999), each of which is incorporated herein by reference in their entirety;

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The compositions of the present invention may be prepared and formulated as emulsions. Emulsions are typically heterogenous systems of one liquid dispersed in another in the form of droplets usually exceeding 0.11 μm in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents-are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporinA into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765). Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-Ser. No.-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 120 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′-isothiocyano-stilbene-2,2′-disulfonic acid (Miyao et al., Antisense Res. Dev., 1995, 5, 115121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

### EXAMPLES

### Example 1

Scheme 1 is the synthetic scheme for monomers and intermediates described in Examples 1-12 and 120.

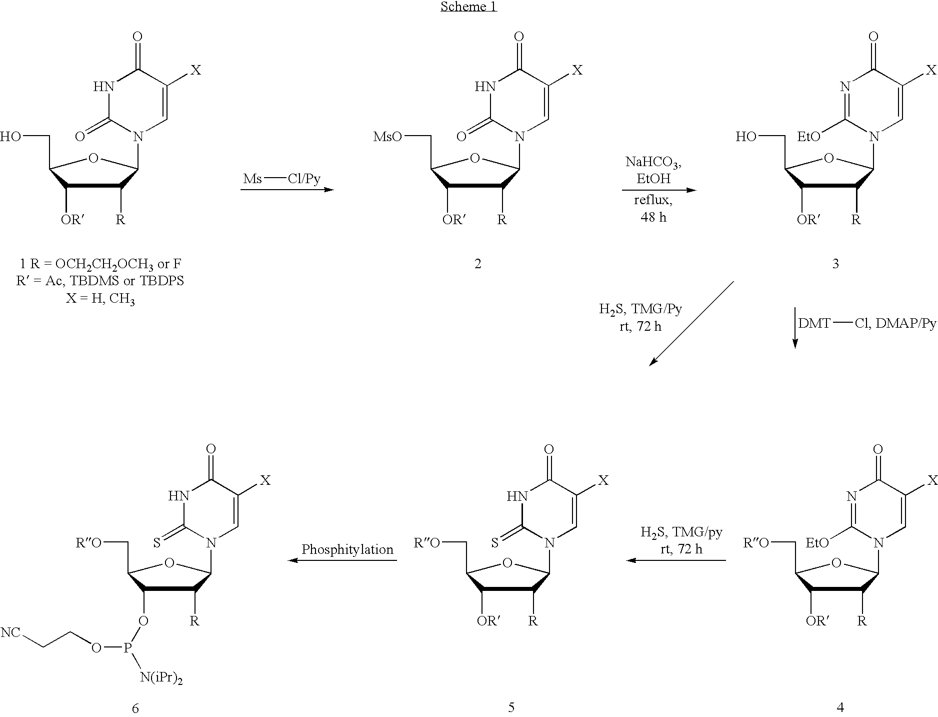

Compound 3 (R=OCH₂CH₂OCH₃, R′=H, X=CH₃, Scheme 1).

Compound 2 (R=OCH₂CH₂OCH₃, R′=OAc, X=CH₃) was prepared from 2′-O-(2-methoxy)ethyl-3′-O-thymidine (prepared as reported, Martin P. Helvetica Chimica Acta, 1995, 78, 486-504) and methanesulfonyl chloride according to standard procedure. Compound 2 (10.0 g, 22.94 mmol) after drying over P₂O₅ under vacuum was refluxed in absolute ethanol (100 mL) in the presence of anhydrous sodium bicarbonate (4.82 g, 57.37 mmol, 2.5 molar eq.) under argon for 30 h. Progress of the reaction was monitored by TLC. After cooling to room temperature, reaction mixture was diluted with ethyl acetate and the precipitated sodium salt was removed by filtration. Filtrate was concentrated to a white solid and was purified by silica gel column chromatography: eluent, 4% MeOH in DCM, to obtain compound 3 (5.95 g, 75.4%) as a white solid. ¹H NMR (200 MHz, DMSO-d₆): δ 7.93-7.92 (d, 1H), 5.79-5.77 (d, 1H, J=4.40 Hz), 5.23-5.18 (t, 1H, exchangeable with D₂O), 5.09-5.06 (d, 1H, exchangeable with D₂O), 4.40-4.28 (m, 2H), 4.15-4.06 (m, 1H), 4.01-3.96 (t, 1H), 3.90-3.84 (m, 1H), 3.75-3.53 (m, 4H), 3.45-3.40 (t, 2H), 3.32 (s, 1H exchangeable with D₂O), 3.20 (s, 3H), 1.78-1.77 (d, 3H), 1.33-1.25 (t, 3H). ¹³C NMR (50 MHz, DMSO-d₆): δ 170.2, 154.6, 133.8, 115.8, 87.6, 85.1, 82.0, 71.2, 69.2, 68.0, 64.2, 60.1, 58.1, 14.0, 13.4. FAB-Glycerol MS: Calc. for C₁₅H₂₄N₂O₇ 344.16, Found 345 (MH⁺).

### Example 2

Compound 4 (R=OCH₂CH₂OCH₃, R′=H, R″=DMT, X=CH₃, Scheme 1). Compound 3 (5.60 g, 16.279 mmol), after drying over P₂O₅ under vacuum, was reacted with DMT-Cl (6.06 g, 17.88 mmol, 1.1 molar eq.) in the presence of DMAP (0.20 g, 1.64 mmol) in anhydrous pyridine under argon atmosphere at ambient temperature for 4 h. Removed pyridine from the reaction mixture and the residue suspended in ethyl acetate (50 mL) was washed with saturated sodium bicarbonate solution (20 mL) and water (20 mL). The organic phase was evaporated to dryness and the residue loaded on a silica gel column was eluted out with 4% MeOH in DCM to obtain compound 4 (9.5 g, 90.33%) as a white foam. ¹H NMR (200 MHz, DMSO-dc): δ 7.61(s, 1H), 7.41-7.24 (m, 9H), 6.91-6.87 (d, 4H), 5.80-5.78 (d, 1H, J=4.0 Hz), 5.23-5.20 (d, 1H, exchangeable with D₂O), 4.39-4.21 (m, 3H), 4.15-4.11 (m, 1H), 4.02 (bm, 1H), 3.76-3.49 (m, 8H), 3.49-3.44 (t, 2H), 3.31-3.21 (m, 5H), 1.38 (s, 3H), 1.32-1.25 (t, 3H). ¹³C NMR (50 MHz, DMSO-d₆): δ 170.1, 158.2, 154.6, 144.6, 135.3, 135.1, 133.3, 129.8, 127.9, 127.7, 126.8, 116.0, 113.3, 88.2, 86.0, 83.1, 81.8, 71.4, 69.5, 68.6, 64.3, 62.8, 58.2, 55.1, 14.0, 12.8. FAB MS: Calc. for C₃₆H₄₂N₂O₉ 646.29, Found 647 (MH⁺).

### Example 3

Compound 5 (R=OCH₂CH₂OCH₃, R′=H, R″=DMT, X32 CH₃, Scheme 1). Compound 4 (6.0 g, 9.28 mmol, R=OCH₂CH₂OCH₃, R′=H, R″=DMT, X=CH₃, Example 1) after thorough drying over P₂O₅ under vacuum was placed in a 250 mL round bottom flask (RB) under argon atmosphere and cooled over a freezing bath. A solution of anhydrous 1,1,3,3-tetramethylguanidine (TMG, 11.7 mL, 93.25 mmol) in pyridine (100 mL) was flushed with argon and cooled to 0° C. over a freezing bath. After cooling the pyridine solution was saturated with hydrogen sulfide for 45 min by maintaining the temperature of the bath below 0° C. The solution was then transferred into the pre-cooled flask containing compound 5 under argon pressure. Temperature of the flask was slowly brought up to room temperature and stored for 72 h. H₂S was gently flushed out into a chlorox bath and then pyridine was removed from the reaction mixture under vacuum. Residue suspended in ethyl acetate was subjected to water wash followed by standard workup. The desired product was purified by column chromatography using ethyl acetate and hexane (1:1) as eluent to yield compound 4 as a white foamy solid (4.03 g, 66.2%). ¹H NMR (200 MHz, DMSO-d₆): δ 12.64 (bs, 1H, exchangeable with D₂O), 7.61 (s, 1H), 7.38-7.20 (m, 9H), 6.89-6.84 (d, 4H), 6.61-6.59 (d, 1H, J=3.4 Hz), 5.13-5.10 (d, 1H, exchangeable with D₂O), 4.28-4.20 (m, 1H), 4.08-3.96 (m, 2H), 3.90-3.69 (m, 8H), 3.51-3.46 (t, 2H), 3.30-3.20 (bm, accounted for 14H, 5H+water from the solvent), 1.31 (s, 3H). ¹³C NMR (50 MHz, DMSO-d₆): δ 174.8, 160.6, 158.2, 144.6, 136.0, 135.3, 135.0, 129.8, 128.0, 127.7, 126.9, 115.3, 113.3, 91.5, 86.0, 82.8, 82.1, 71.4, 69.9, 68.7, 62.5, 58.2, 55.1, 11.9. FAB-NBA MS: Calc. for C₃₄H₃₈N₂O₈S 634.23, Found 635 (MW). FAB-NBA/LiCl M.⁷Li⁺ 641. HRMS: Calc. for C₃₄H₃₈N₂O₈S. ⁷Li 641.250893, Found 641.252500.

### Example 4

Compound 3 (R=OCH₂CH₂OCH₃, R′=—Si[TBDP], R″=OH, X=CH₃, Scheme 1). Compound 2 (R=OCH₂CH₂OCH₃, R′=OSiTBDP, X=CH₃) was prepared from 2′-O(2-methoxy)ethyl-3′-O-(t-butyldiphenyl)silyl-thymidine and methanesulfonyl chloride according to standard procedure. Compound 2 (4.7 g, 7.44 mmol) was refluxed with anhydrous sodium bicarbonate (950 mg, 11.31 mmol, 1.52 molar eq.) in absolute ethanol under argon for 48 h, followed by standard workup and purification (silica gel column chromatography: eluent 2% MeOH in DCM) as reported in Example 1, to obtain compound 3 (3.0 g, 69.3%) as a white foam. ¹H NMR (200 MHz, DMSO-d₆): δ 7.80 (s, 1H), 7.70-7.65 (m, 4H), 7.61-7.57 (m, 6H), 5.90-5.88 (d, 1H, J=5.0 Hz), 5.22-5.17 (t, 1H, exchangeable with D₂O), 4.33-4.23 (m, 3H), 3.99-3.97 (bm, 1H), 3.68-3.55 (m, 2H), 3.423.17 (m, 5H), 3.17 (s, 3H), 1.74-1.73 (d, 3H), 1.29-1.22 (t, 3H), 1.04 (s, 9H). ¹³C NMR (50 MHz, DMSO-d₆): δ 170.0, 154.5, 135.5, 135.3, 133.5, 132.9, 132.8, 130.0, 129.9, 127.8, 127.7, 115.8, 86.9, 85.0, 81.3, 71.1, 70.6, 69.0, 64.2, 59.8, 58.2, 26.7, 18.9, 13.9, 13.4. FAB-NBA MS Calc. for C₃₁H₄₂N₂O₇Si 582.28, Found 583 (MH⁺).

### Example 5

Compound 5 (R=OCH₂CH₂OCH₃, R′=TBDPS, R″=H, X=CH₃, Scheme 1). Compound 5 (as specified) was prepared from compound 3 (0.4 g, 0.69 mmol, from Example 4) and TMG-H₂S as described in Example 3. Yield 0.25 g, 63.8%. ¹H NMR (200 MHz, DMSO-d₆): δ 12.59 (s, 1H, exchangeable with D₂O), 7.97 (s, 1H), 7.71-7.57 (m, 4H), 7.48-7.32 (bm, 6H), 6.81-6.79 (d, 1H, J=4.4 Hz), 5.29 (bt, 1H), 4.32-4.27 (bt, 1H), 4.01 (bs, 1H), 3.79-3.59 (bm, 2H), 3.38-3.20 (m, 5H), 3.16 (s, 3H), 1.76 (s, 3H), 1.04 (s, 9H).

### Example 6

Compound 3 (R=OCH₂CH₂OCH₃, R′=TBDPS X=H, Scheme 1). Compound 2 (R=OCH₂CH₂OCH₃, R′=TBDPS, X=H) was prepared from 2′-O-(2-methoxy)ethyl-3′-O-(tbutyldiphenyl)silyl-uridine and methanesulfonyl chloride according to standard procedure. Compound 2 (4.186 g, 6.77 mmol) was refluxed with anhydrous sodium bicarbonate (1.14 g, 13.57 mmol, 2 molar eq.) in absolute ethanol under argon for 60 h, followed by standard workup and purification (silica gel column chromatography: eluent 5% MeOH in DCM) as reported in Example 1, to obtain compound 3 (2.2 g, 57.2%) as a white foam. ¹H NMR (200 MHz, DMSO-d₆): δ 7.95-7.91 (d, 1H, J=7.6 Hz), 7.70-7.56 (m, 4H), 7.50-7.36 (m, 6H), 5.89-5.87 (d, 1H, J=4.4 Hz), 5.82-5.78 (d, 1H, J=7.8 Hz), 5.20-5.15 (t, 1H, exchangeable with D₂O), 4.36-4.23 (m, 3H), 4.00-3.98 (bm, 1H), 3.64-3.57 (m, 2H), 3.43-3.21 (m, 22H, accounts for 5H and water present in the solvent), 3.13 (s, 3H), 1.29-1.22 (t, 3H), 1.04 (s, 9H). ¹³C NMR (50 MHz, DMSO-d₆): δ 169.5, 154.8, 137.8, 135.5, 135.3, 132.9, 132.8, 130.0, 129.9, 127.9, 127.7, 107.9, 87.2, 85.0, 81.5, 71.1, 70.5, 69.0, 64.3, 59.7, 58.2, 26.7, 19.0, 13.8.

### Example 7

Compound 5 (R=OCH₂CH₂OCH₃, R′=TBDPS, R″=H, X=H, Scheme 1). Compound 5 (as specified) was obtained from compound 3 (2.15 g, 3.79 mmol, from Example 6) as described in Example 3. White solid, 1.60 g (76.0% yield). ¹H NMR (200 MHz, DMSO-d₆): δ 12.64 (s, 1H exchangeable with D₂O), 8.06-8.02 (d, 1H, J=8.2 Hz), 7.71-7.58 (m, 4H), 7.50-7.36 (m, 6H), 6.84-6.82 (d, 1H, J=4.6 Hz), 6.00-5.94 (dd, 1H, J′=8.2, J″=1.8 Hz), 5.26-5.22 (t, 1H, exchangeable with D₂O), 4.34-4.29 (t, 1H), 4.00-3.96 (bm, 1H), 3.66-3.54 (m, 3H, accounts for 2H and water from the solvent), 3.34-3.17 (m, 5H), 3.15 (s, 3H), 1.04 (s, 9H). ¹³C NMR (50 MHz, DMSO-d₆): δ 176.2, 159.3, 140.6, 135.5, 135.4, 132.9, 132.8, 130.0, 129.9, 127.8, 127.7, 106.8, 90.0, 85.0, 81.6, 71.2, 70.9, 69.4, 59.6, 58.2, 26.7, 18.9. FAB-NBA MS Calc. for C₂₈H₃₆N₂O₆SiS: 556, Found: 557 (MH⁺).

### Example 8

Compound 6 (R=OCH₂CH₂OCH₃, X=CH₃, R″=DMT, Scheme 1). Compound 5 (0.33 g, 0.52 mmol) from Example 3 was dried over anhydrous P₂O₅ under vacuum along with tetrazole diisopropylammonium salt (0.09 g, 0.53 mmol) overnight and then suspended in anhydrous MeCN (5 mL) under argon atmosphere. 2-Cyanoethyl tetraisopropylphosphrodiamidite (0.33 mL, 1.04 mmol) was added into the suspension at ambient temperature and stirred for 6 h. Removed MeCN from the reaction mixture, residue in ethyl acetate (20 mL) was washed with saturated sodium bicarbonate followed by standard workup. Compound 6 was purified by column chromatography, eluent: ethyl acetate/hexane (1:1) to yield 0.41 g (94.4% yield). 31P NMR (80.95 MHz, CDCl₃): δ 151.6, 150.74. HRMS Calc. for C₄₃H₅₆N₄O₉PS 835.350565, Found 835.351090.

### Example 9

Compound 2 (R=F, R′=Ac, X=Me, Scheme 1): Compound 1 (R=F, X=Me, 750 mg, 2.48 mmol, prepared as reported, Condington, J. F. et. al. J. Org. Chem. 1964, 29, 558-564) was treated with methanesulfonylchloride (0.4 mL, 5.16 mmol) in pyridinedichoromethane (1:1, 5 mL) at −20° C. bath temperature for 1 hour. Solvents were removed from the reaction mixture and the residue, suspended in water (10 mL), was extracted with ethylacetate (25 mL), washed with saturated NaHCO₃ solution (10 mL) and brine (10 mL). The product extracted was purified by flash column chromatography to obtain the desired compound 2 as a white foam, eluent: 5% MeOH in dicholoromethane; yield: 930 mg, (98.5%). ¹H NMR (200 MHz, DMSO-d₆): δ 11.47 (s, exchangeable with D₂O), 7.57 (s, 1H), 5.96-5.84 (dd, 1H, H1′, J′=2.20, J″=21.80 Hz), 5.65-5.62 (m, 0.5H), 5.37-5.21 (m, 1.5H), 4.54-4.34 (m, 3H), 3.20 (s, 3H), 2.11 (s, 3H), 1.77 (s, 3H).

### Example 10

Compound 4 (R=F, R′=H, R″=DMT, X=Me, Scheme 1): Compound 2 (900 mg, 2.37 mmol) obtained from Example 9 was mixed with anhydrous NaHCO₃ (500 mg, 5.95 mmol) and dried over P₂O₅ under vacuum overnight. The mixture was then suspended in absolute ethanol (200 proof, 10 mL) and refluxed as reported in Example 1 to obtain the 2-O-ethyl derivative, which was subsequently reacted with DMT-Cl (800 mg, 2.36 mmol) in the presence catalytic amount of DMAP (30 mg, 0.25 mmol) in anhydrous pyridine as reported in Example 2 to obtain the desired compound 4. The product was purified by flash chromatography; eluent: DCM/EtOAc (1:4); yield: 400 mg (28.6%). ¹H NMR (200 MHz, CDCl₃): δ 7.64-7.64 (d, 1H), 7.43-7.15 (m, 9H), 6.87-6.81 (m, 4H), 6.11-6.02 (dd, 1H, H1′, J′=2.50 and J″=15.30 Hz), 5.21-5.17 (m, 0.5H, H2′), 4.95-4.91 (m, 0.5H, H2′), 4.60-4.46 (m, 3H), 4.18-4.14 (m, 1H), 3.79-3.65 (m, 6H), 3.65-3.43 (m, 2H), 1.51 (s, 3H), 1.38-1.29 (t, 3H).

### Example 11

Compound 5 (R=F, R′=H, R″=DMT, X=Me, Scheme 1): Compound 4 (300 mg, 0.51 mmol) obtained from Example 10 was taken in a 25 mL RB and dried over P₂O₅ under vacuum overnight, sealed the flask under argon and cooled over an ice bath under argon pressure. Anhydrous pyridine (10 mL) was placed on an ice bath under argon atmosphere and after cooling the dry H₂S gas was bubbled through the solvent for 30 min. The pyridine-H₂S solution was then transferred into the flask containing compound 4 under cold. The reaction mixture sealed and placed on 60° C. oil bath for 72 h. Removed pyridine and the residue taken in EtOAc (25 mL) was washed with water and bicarbonate solution. After evaporation of EtOAc, the residue was subjected to flash column chromatography to obtain the desired 2-thio-2′-fluoro nucleoside 5 as a white solid. Eluent: Hexane:EtOAc 3:1; yield: 140 mg (47.65). ¹H NMR (200 MHz, CDCl₃+DMSO-d₆+D₂O): δ 7.95 (s, 1H), 7.39-7.27 (m, 9H), 6.87-6.83 (m, 4H), 6.71-6.63 (d, 1H, H1′, J=15.60 Hz), 5.28 (bs, 0.5, H2′), 5.01 (bs, 0.5H, H2′), 4.60-4.42 (bm, 1H), 4.24-4.19 (bm, 1H), 3.80 (s, 6H), 3.69-3.47 (m, 2H), 1.27 (s, 3H).

### Example 12

Compound 6 (R=F,R″=DMT, X=Me, Scheme 1): Compound 5 from Example 11 is phosphitylated as reported in Example 8 to obtain the desired phosophoramidite 6.

### Example 13

Schemes 2a is the synthetic scheme for monomers and intermediates described in Examples 13-24 and 27.

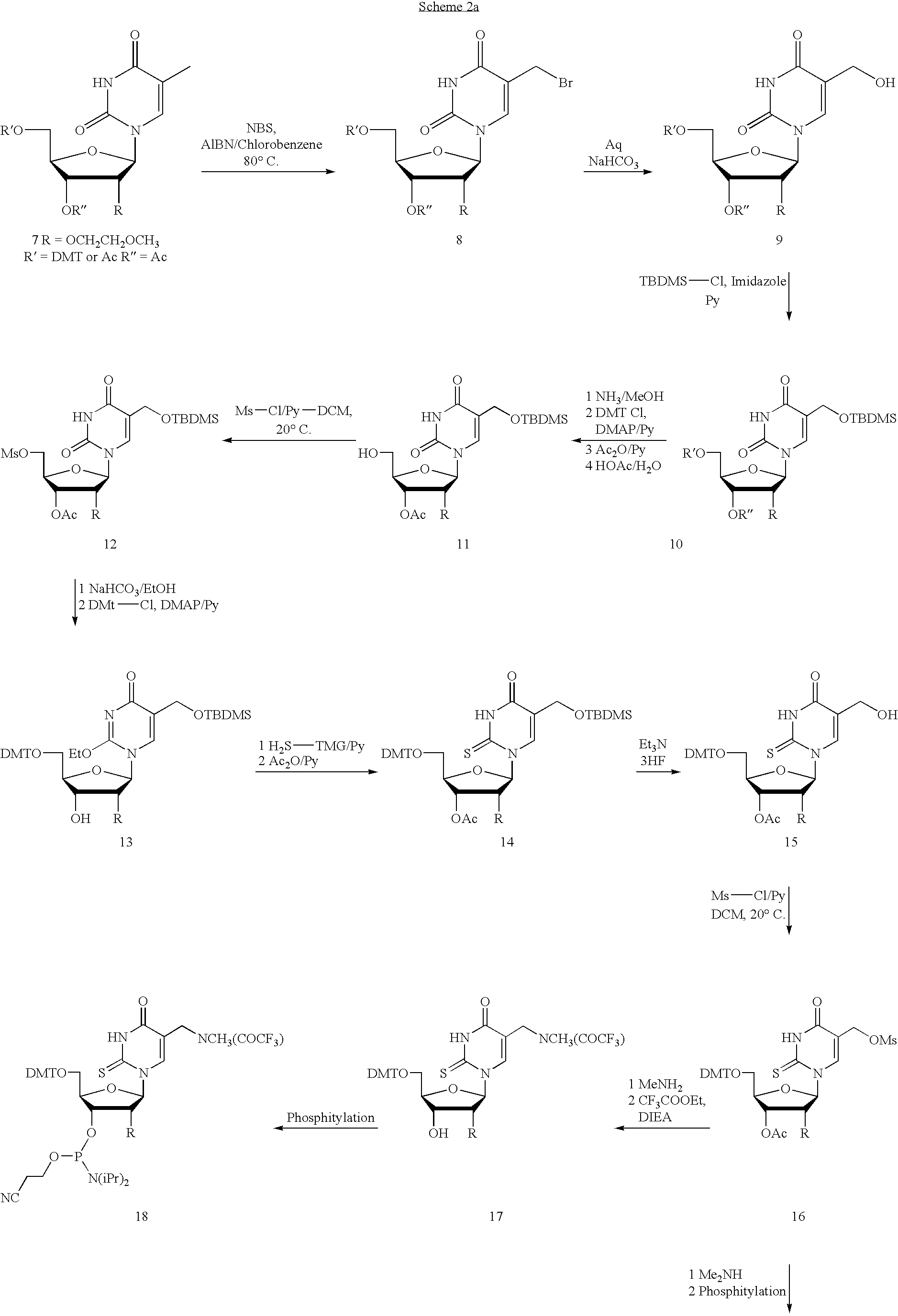

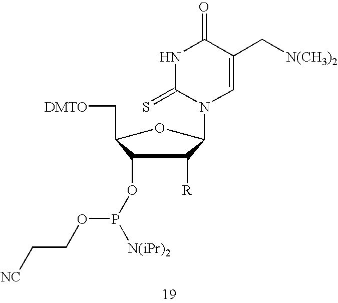

Compound 8 (R′, R″=Ac, R=OCH₂CH₂OCH₃, Scheme 2a): Compound 7 (16.5 g, 41.25 mmol, R′, R″=Ac, R=OCH₂CH₂OCH₃, Example 2a) was co-evaporated with chlorobenzene and subsequently redissolved in chlorobenzene (200 mL). The solution was thoroughly deoxygenated by gentle flushing of anhydrous argon through the solution for 10 min. Finally powdered NBS (11.02 g, 61.91 mmol, 1.5 mol eq.) was added into the solution under argon. The reaction mixture was again flushed with argon for 5 min and then placed over a pre-heated oil bath of 80° C. under constant stirring. AIBN (100 mg, 0.6089; 1 mol %) was added into the hot reaction mixture, the pale golden yellow reaction mixture turned to brown after the addition of AIBN and the brown coloration disappeared after ten min. The stirring was continued for 30 minute and the mixture turned to brown again. TLC after 15 min and after 30 min of addition of AIBN showed about 60% product formation. The reaction mixture was cooled to room temperature and the precipitated succinimide was filtered off, washed with chlorobenzene. The filtrate after concentration under vacuum was directly loaded on column of silica gel and the bromo derivative 8 was eluted out with ethyl acetate/hexane (1:1) to obtain 11.05 g (55.6%) as a white solid. ¹H NMR (200 MHz, DMSO-d₆): δ 11.96 (s, 0.2H, exchangeable with D₂O, minor rotamer), 11.71 (s, 0.6H, exchangeable with D₂O, major rotamer), 8.03 (s, 0.75H, major rotamer), 7.96 (s, 0.25H, minor rotamer), 5.83-5.78 (m, 1H), 5.17-5.10 (m, 1H), 4.38-4.19 (m, 6H), 3.66-3.48 (m, 3H), 3.40-3.33 (m, 2H), 3.19-3.16 (m, 3H), 2.08-2.04 (m, 6H). ¹H NMR (200 MHz, DMSO-d₆+D₂O; after 2 h): δ 7.52 (s, 1H), 5.88-5.85 (d, 1H, J=6.2 Hz), 5.195.15 (m, 1H), 4.31-4.15 (m, 6H), 3.57-3.51 (m, 2H), 3.33-3.30 (m, 2H), 3.15-3.14 (m, 3H), 2.09-2.07 (m, 6H).

### Example 14

Compound 9 (R′, R″=Ac, R=OCH₂CH₂OCH₃, Scheme 2a): Compound 8 (4.2 g, 8.77 mmol) in 20 mL ethyl acetate was mixed with 5 mL of 10% aq. NaHCO₃ and stirred at ambient temperature for 3 h. After 3 h, the hydroxy compound formed was repeatedly extracted from the aqueous layer with EtOAc (6×25 mL) as the product was soluble in both aqueous and organic phase. Evaporated the organic layer and the residue obtained was subjected to silica gel column chromatography due to mild contamination of succinimide from the NBS reaction (Scheme 9). Eluent: 4% MeOH in DCM; Compound 9: 2.49 g (68.3%, white foam). ¹H NMR (200 MHz, DMSO-d₆): δ 11.47 (s, 1H, exchangeable with D₂O), 7.53 (s, 1H), 5.90-5.87 (d, 1H, J=6.4 Hz), 5.21-5.18 (m, 1H), 5.13-5.08 (t, 1H, exchangeable with D₂O), 4.33-4.16 (m, 6H), 3.59-3.52 (m, 2H), 3.373.31 (m, 2H), 3.17-3.16 (m, 3H), 2.09-2.07 (m, 6H). ¹³C NMR (50 MHz, DMSO-d₆): δ 170.8, 170.2, 162.7, 150.7, 136.2, 115.2, 87.0, 79.6, 79.0, 71.7, 70.9, 70.0, 63.6, 58.4, 56.0, 20.81, 20.79.

### Example 15

Compound 10 (R′, R″=Ac, R=OCH₂CH₂OCH₃, Scheme 2a): Compound 9 (2.3 g, 5.53 mmol), TBDMS-Cl (1.25 g, 8.29 mmol) and imidazole (1.13 g, 16.6 mmol) were stirred in anhydrous pyridine at ambient temperature for overnight. Removed pyridine from the reaction mixture followed by standard workup. The residue obtained was passed through a column of silica gel-to remove excess TBDMS-Cl to obtain compound 10 as a white foam (2.35 g, 80.2%). ¹H NMR (200 MHz, CDCl₃): δ 8.72 (s, 1H, exchangeable with D₂O), 7.45-7.44 (d, 1H), 5.90-5.88 (d, 1H, J=4 Hz), 5.04-4.98 (t, 1H, J′=5.8, J″=6.0 Hz), 4.50-4.49 (m, 2H), 4.41-4.24 (m, 4H), 3.77-3.67 (m, 2H), 3.50-3.45 (t, 2H, J′=4.6, J″=4.4 Hz), 3.31 (s, 3H), 2.15-2.12 (d, 6H), 0.91 (s, 9H), 0.11 (s, 6H).

### Example 16

Compound 11 (R=OCH₂CH₂OCH₃, Scheme 2a): Compound 10 (2.2 g, 4.15 mmol) was subjected to methanolic ammonia treatment at ambient temperature for 4 h. Progress of the deacetylation was monitored by TLC and after complete deprotection, ammonia and methanol were removed under vacuum. The residue was repeatedly evaporated with DCM and then dried over anhydrous P₂O₅ under vacuum. The anhydrous residue was then treated with DMT-CL (1.68 g, 4.96 mmol) and DMAP (120 mg, 0.98 mmol) as reported in Example 2. Acetic anhydride (1 mL, excess) was added into the reaction mixture after overnight treatment with DMT-Cl in pyridine to acetylate 3′-hydroxyl function of the sugar moiety. The reaction mixture was stirred for 4 h. Methanol was added into reaction to quench excess anhydride. Removed pyridine, the residue in ethyl acetate (30 mL) was washed with saturated NaHCO₃ solution. After evaporating ethyl acetate, the solid obtained was dissolved in 80% aqueous acetic acid and stirred at ambient temperature for 4 h. Acetic acid was removed from the reaction mixture under vacuum and the residue in ethyl acetate (40 mL) was washed with water and aqueous bicarbonate solution. Compound 11 was then purified by silica gel column chromatography. Eluent: 4% methanol in DCM, 1.25 g (61.7%, white foam, hygroscopic). ¹H NMR (200 MHz, DMSO-d₆): δ 11.47 (s, 1H, exchangeable with D₂O), 7.80 (s, 1H), 5.92-5.88 (d, 1H, J=6.8 Hz), 5.27-5.18 (m, 2H) [Note: After D₂O exchange: δ 5.24-5.20, m, 1H], 4.33 (s, 2H), 4.24-4.18 (t, 1H), 4.07=4.05 (m, 1H), 3.60-3.49 (m, 4H), 3.34-3.31 (m, 2H), 3.15 (s, 3H), 2.09 (s, 3H), 0.86 (s, 9H), 0.07 (s, 6H).

### Example 17

Compound 12 (Scheme 2a): Compound 11 (1.1 g, 2.25 mmol) was taken in 10 mL of anhydrous DCM-Pyridine (1:1) and stirred at −20° C. Methanesulfonyl chloride (0.5 mL, 6.46 mmol) was added into the stirring solution drop wise and the stirring was continued for 2 h at −20° C. Removed pyridine from the reaction mixture under diminished pressure and standard workup in ethyl acetate was followed. The sulfonate 12 was passed through a column of silica gel; eluent DCM/EtOAc (3:2), to obtain the desired product as a white foam, yield 1.28 g (quantitative). ¹H NMR (200 MHz, CDCl₃): δ 9.18 (s, 1H, exchangeable with D₂O), 7.36 (s, 1H), 5.80-5.78 (d, 1H, H1′, J=4.40 Hz), 5.17-5.11 (t, 1H), 4.51-4.38 (m, 6H), 3.73-3.68 (m, 2H), 3.49-3.45 (m, 2H), 3.31 (s, 3H), 3.07 (s, 3H), 2.16 (s, 3H), 0.93 (s, 9H), 0.13 (s, 6H). ¹³C NMR (200 MHz, CDCl₃): 170.1, 162.1, 150.0, 137.3, 115.0, 91.5, 79.6, 79.3, 72.0, 71.0, 70.5, 67.6, 58.9, 58.0, 37.7, 25.9, 20.6, 18.4.

### Example 18

Compound 13 (Scheme 2a): Compound 12 (1.25 g, 2.2 mmol) was mixed with anhydrous NaHCO₃ (470 mg, 5.59 mmol) and dried over P₂O₅ under vacuum overnight. The mixture was then suspended in absolute ethanol (200 proof, 10 mL) and refluxed as reported in Example 1 to obtain the 2-O-ethyl derivative, which was subsequently reacted with DMT-Cl (750 mg, 2.21 mmol) in the presence catalytic amount of DMAP (27 mg, 0.22 mmol) in anhydrous pyridine as reported in Example 2 to obtain the desired compound 13. The product was purified by flash chromatography; eluent: EtOAc; yield: 1.02 g (59.5%). ¹H NMR (200 MHz, DMSO-d₆): δ 7.46-7.20 (m, 10H), 6.88-6.83 (d, 4H), 5.83-5.82 (d, 1H, H1′, J=1.80 Hz), 5.25 (bs, 1H, exchangeable with D₂O), 4.38-4.16 (m, 3H), 4.02-3.92 (bm, 4H), 3.72-3.65 (m, 8H), 3.47-3.42 (m, 2H), 3.31-3.19 (m, 7H, became 5H after D₂O exchange, the additional 2H could be due to the presence of water from DMSO-d₆ or from the compound), 1.31-1.24 (t, 3H), 0.73 (s, 9H), −0.07 (s, 3H), −0.10 (s, 3H). ¹³C NMR (200 MHz, DMSO-d₆): δ 168.9, 158.3, 155.0, 144.8, 135.6, 135.4, 134.3, 129.9, 128.1, 127.8, 127.0, 119.5, 113.4, 88.9, 86.0, 82.9, 81.7, 71.6, 69.7, 69.1, 64.9, 63.5, 58.7, 58.4, 55.2, 25.9, 18.1, 14.1, -5.3, -5.4.

### Example 19

Compound 14 (Scheme 2a): Compound 13 (950 mg, 1.22 mmol) was reacted with H₂S in the presence of TMG (1.54 mL, 12.27 mmol) in anhydrous pyridine as reported in Example 3 to obtain the corresponding 2-thio derivative. The 2-thioderivative after workup was purified by flash column chromatography. Eluent: 30% EtOAc in hexane, yield: 760 mg (81.3%, white solid). ¹H NMR (200 MHz, DMSO-d₆): δ 12.78 (s, 1H, exchangeable with D₂O), 7.54 (s, 1H), 7.42-7.24 (m, 9H), 6.89-6.85 (d, 4H), 6.68 (s, 1H, H1′), 5.22-5.20 (d, exchangeable with D₂O), 4.16-3.96 (m, 4H), 3.86-3.72 (m, 8H), 3.513.45 (m, 2H), 3.31-3.15 (m, 6H), 0.75 (s, 9H), −0.06-0.10 (m, 6H). Acetylation of the compound thus obtained with acetic anhydride in pyridine yields the desired product 14.

### Example 20

Compound 15 (Scheme 2a): Treatment of compound 14 with triethylamine trihyrdofluoride in THF yields compound 15.

### Example 21

Compound 16 (Scheme 2a): Compound 15 is reacted with methanesulfonyl chloride as reported in Example 17 to obtain compound 16.

### Example 22

Compound 17 (Scheme 2a): Compound 16 is stirred with methylamine at low temperature and subsequently treated with ethyl trifluoroacetate in the presence of DIEA to obtain compound 17.

### Example 23

Compound 18 (Scheme 2a): Phosphitylation of compound 17 under the conditions as reported in Example 8 yields the phosphoramidite 18.

### Example 24

Compound 19 (Scheme 2a): Treatment of compound 16 with dimethylamine followed phosphitylation as reported in Example 8 to obtain the required amidite 19.

### Example 25

Scheme 2b is the synthetic scheme for monomers and intermediates described in Examples 25 and 26.

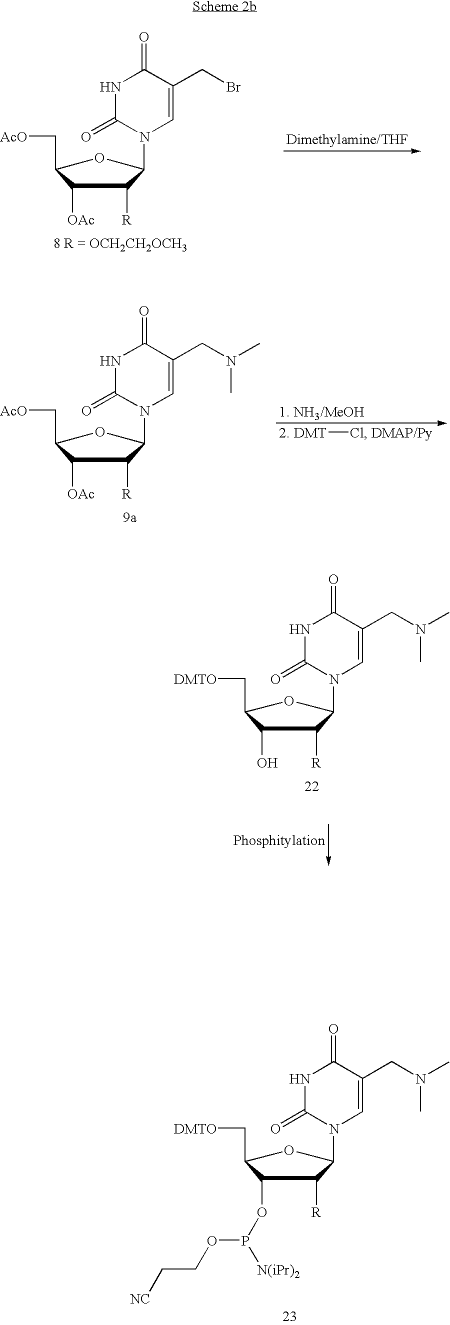

Compound 22 (Scheme 2b): Compound 8 (R′, R″=acetyl, Example 2a, 5.2 g, 10.86 mmol, purity about 90%) was treated with 2M dimethylamine in anhydrous THF (30 mL) for 10 minute at ambient temperature. Removed excess amine and THF in vacuo, and the residue were extracted into ethyl acetate. Removed the solvent in vacuo and dried under vacuum overnight to obtain compound 9a. ¹H NMR (200 MHz, DMSO-d₆+D₂O): δ 7.53 (s, 1H), 5.83-5.80 (d, 1H, H1′, J=6.20 Hz), 5.18-5.14 (m, 1H), 4.33-4.19 (m, 4H), 3.563.51 (m, 2H), 3.35-3.15 (m, 2H), 3.15-3.12 (m, 5H), 2.14 (s, 6H), 2.06-2.05 (d, 6H).

Compound 9a was treated with methanolic ammonia for 4 h at ambient temperature to remove the acetyl protection. After ammonia treatment the residue was dried over P₂O₅ under vacuum overnight. The dried residue was treated with DMT-Cl (3.4 g, 10.03 mmol) and DMAP (25 mg, 0.20 mmol) in anhydrous pyridine under argon atmosphere to obtain compound 22. After removing pyridine from the reaction mixture the product was extracted into ethyl acetate (50 mL). The separation of aqueous and organic phase took longer time due to the presence of tertiary amino moiety in the product. The aqueous layer was re extracted with dichloromethane (50 mL) and combined the organic phase, evaporated to dryness in vacuo. The product was purified by flash column chromatography to obtain compound 22 as a yellowish solid. Eleunt: EtOAc/MeOH (1:1); isolated yield: 1.3 g (18.1%). ¹H NMR (200 MHz, DMSO-d₆): δ 11.52 (bs, exchangeable with D₂O), 7.54 (s, 1H), 7.41-7.15 (bm, 9H), 6.89-6.85 (d, 4H), 5.84-5.82 (bd, 1H, H1′, J=4.00 Hz), 5.16-5.13 (d, 1H, exchangeable with D₂O), 4.17-4.00 (bm, 3H), 3.72 (bs, 8H), 3.49-3.45 (bm, 2H), 3.22-3.03 (bm, 6H), 2.86-2.84 (bd, 1H), 2.04 (s, 6H). ¹³C NMR (50 MHz, DMSO-d₆): δ 163.9, 158.8, 150.8, 145.1, 140.6, 136.0, 135.9, 130.5, 128.6, 128.4, 127.7, 113.9, 109.1, 87.9, 86.5, 83.4, 81.7, 71.9, 70.0, 69.3, 63.7, 58.8, 55.7, 55.2, 53.9, 44.2.

### Example 26

Compound 23 (Scheme 2b): Compound 23 was prepared from compound 22 (1.2 g, 1.82 mmol), 2-cyanoethyl tetraisopropylphosphorodiamidite (1 mL, 3.15 mmol) and tetrazole diisopropylammonium salt (310 mg, 1.81 mmol) as reported in Example 8. Due to the presence of the dimethylaminomethyl moiety in the amidite, standard chroamtographic purification was not successful. So the amidite was initially precipitated from dichloromethane-hexane and subsequently purified by flash column chromatography using 30% acetone in dichloromethane as eluent under anhydrous condition to obtain the pure phosphoramidite 23 as a pale yellow solid. Isolate yield 1.16 g (77.0%). ³¹P NMR (80.96 MHz, CDCl₃): δ 151.36, 151.14.

### Example 27

Compound 8 (R=O(CH₂)₂OCH₃, R′=Ac, R″=DMT, Scheme 2a): 5-Me-2′-O-MOE-3′-O-acetyl-5′-O-DMT-U (7) is reacted with NBS under free radical conditions as reported in Example 13 to obtain the corresponding bromo compound 8.

### Example 28

Scheme 3 is the synthetic scheme for monomers and intermediates described in Examples 28-30.

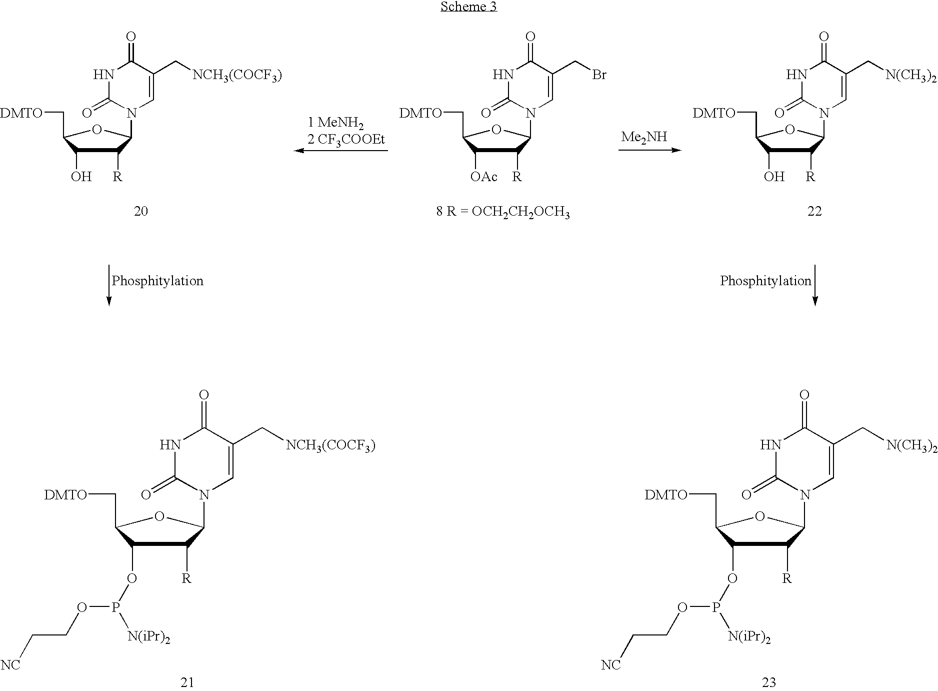

Compound 20 (R=O(CH₂)₂OCH₃, Scheme 3): Reaction of compound 8 from Example 25 with anhydrous methylamine in TUF followed by ethyl trifluoroacetate in the presence of DIEA gives compound 20.

### Example 29

Compound 21 (R=O(CH₂)₂OCH₃, Scheme 3): Phosphitylation of compound 20 under the conditions reported in Example 8 yields compound 21.

### Example 30

Compound 23 (R=O(CH₂)₂OCH₃, Scheme 3): Treatment of the bromo compound 8 with dimethylamine followed by phosphitylation as reported in Examples 25 and 26 yields compound 23.

### Example 31

Scheme 4 is the synthetic scheme for monomers and intermediates described in Examples 31-34.

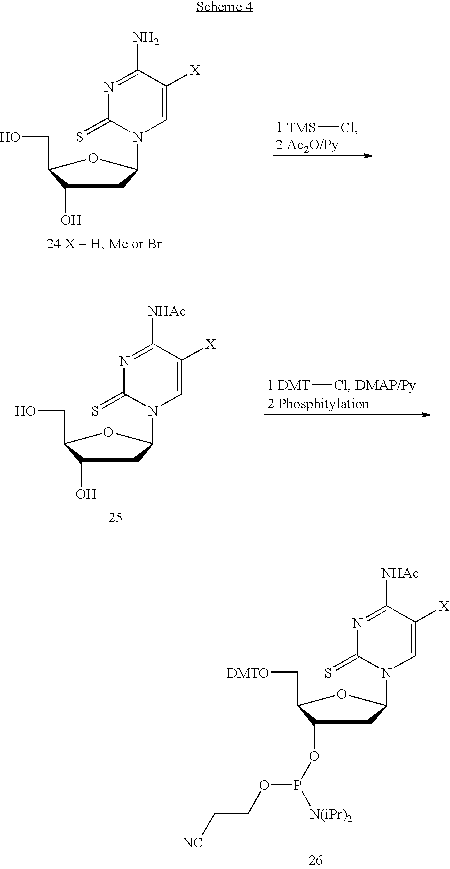

Compound 25 (X=Me, Scheme 4): Compound 24 is prepared from 5-methyl-2-thiocytosine and 1-chloro-3,5-di-O-p-toluyil-2-deoxyribofuranose as reported in the literature (Bretner et. al., Nucleosides and Nuceotides, 1995, 14, 657-660). Transient protection of the sugar hydroxyl functions of compound 24 with TMS-Cl and subsequent reaction of the silylated derivative with acetic anhydride in pyridine gives the N-acylated derivative 25.

### Example 32

Compound 26 (X=Me, Scheme 4): Reaction of compound 25 with DMT-Cl in the presence of DMAP as reported in Example 2 gives the corresponding 5′-O-DMT protected nucleoside. The 3′-hydroxyl of which is phosphitylated under the conditions reported in Example 8 to obtain the phosphoramidite 26.

### Example 33

Compound 26 (X=H, Scheme 4): Phosophoramidite 26 of 2-mercapto-2′-deoxycytidine is prepared from 2-thiocytosine and 1-chloro-3,5-di-O-p-toluyil-2-deoxyribofuranose as reported in Examples 31 and 32.

### Example 34

Compound 26 (X=Br, Scheme 4): Phosphoramidite of 5-Bromo-2-thiocytidine 26 is prepared from corresponding 5-bromo-2-mercaptocytosine and 1-chloro-3,5-di-O-p-toluyil-2-deoxyribofuranose as reported in Examples 31 and 32.

### Example 35

Scheme 5 is the synthetic scheme for monomers and intermediates described in Examples 35-41.

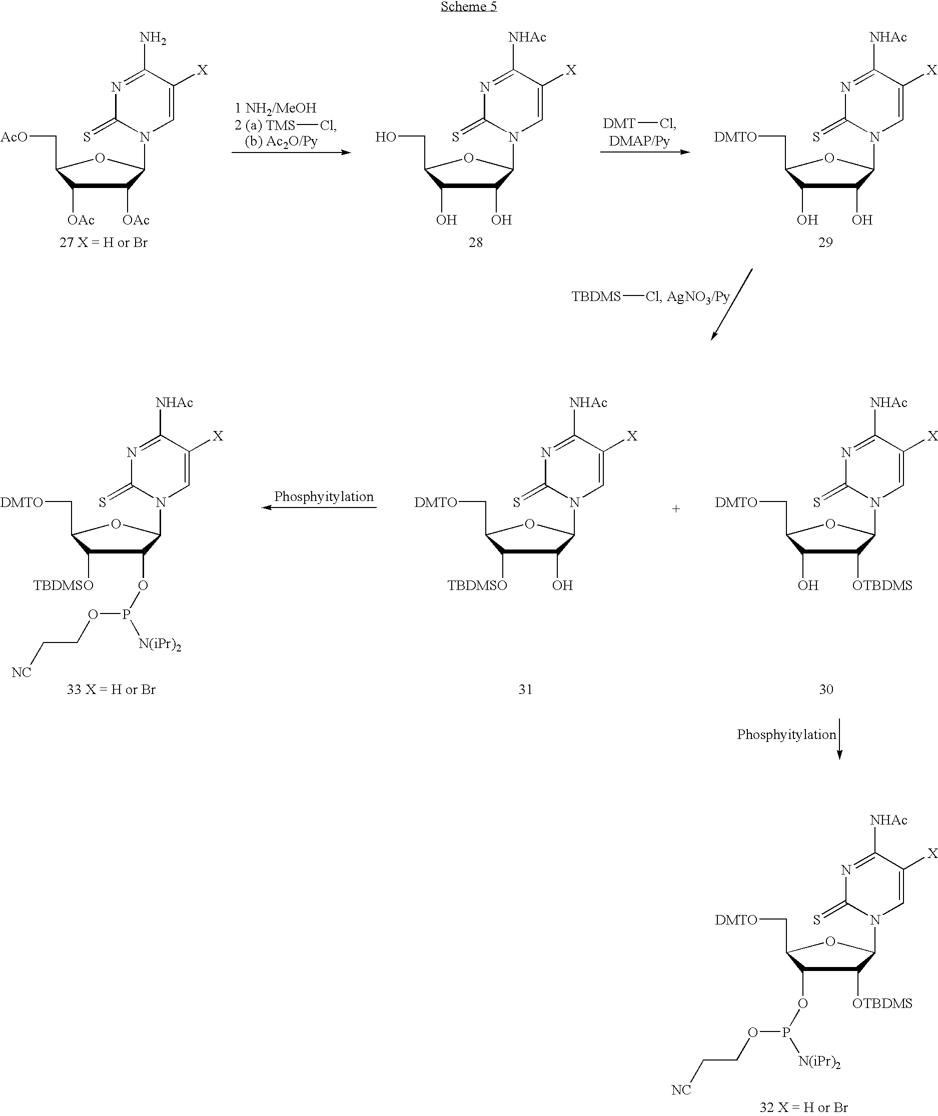

Compound 28 (X=H, Scheme 5): Compound 25 as defined is obtained from 2-mercapto-cytosine and 1,2,3,5-tetra-O-acetyl-p-D-ribofuranose as reported in the literature (Rajeev and Broom, Org. Lett., 2000, 2, 3595-3598). Compound 27 is stirred with methanolic ammonia at 0° C. to deblock the acetyl protection from the sugar moiety of compound 27. After thorough drying of the unprotected nucleoside the hydroxyl functions are transiently protected as its triemethylsilyl derivative by treatment with TMS-CI. The sugar-protected nucleoside thus obtained is reacted with acetic anhydride in pyridine to obtain compound 28.

### Example 36

Compound 29 (X=H, Scheme 5): Compound 28 is reacted with DMT-Cl as reported in Example 2 to obtain compound 29.

### Example 37

Compound 30 and 31 (X=H, Scheme 5): Reaction of compound 29 with TBDMS-Cl in THF-pyridine in the presence of AgNO₃ yields mostly the 2′-O-TBDMS derivative 30 along with its 3′-O-TBDMS derivative 31 (Milicki et. al., Tetrahedron, 1999, 55, 6603-6622). Both the isomers are separated by silica gel column chromatography.

### Example 38

Compound 32 (X=H, Scheme 5): Phosphitylation of compound 30 under the conditions reported in Example 8 yields the phosphoramidite 32.

### Example 39

Compound 33 (X=H, Scheme 5): Compound 31 is phosphitylated as reported in Example 8 to obtain compound 33.

### Example 40

Compound 32 (X=Br, Scheme 5): The 5-bromo-2-thio derivative of cytidine phosphoramidite is prepared from the corresponding 5-bromo-2-thiocytosine and 1,2,3,5-tetra-O-acetyl-p-D-ribofuranose as reported in Examples 35, 36, 37 and 38.

### Example 41

Compound 33 (X=Br, Scheme 5): The desired phosphoramidite 33 is obtained from corresponding 5-halo/H-2-mercaptocytosine and 1,2,3,5-tetra-O-acetyl-β-D-ribofuranose as reported in Examples 35, 36, 37 and 38.

### Example 42

Scheme 6 is the synthetic scheme for monomers and intermediates described in Examples 42-47.

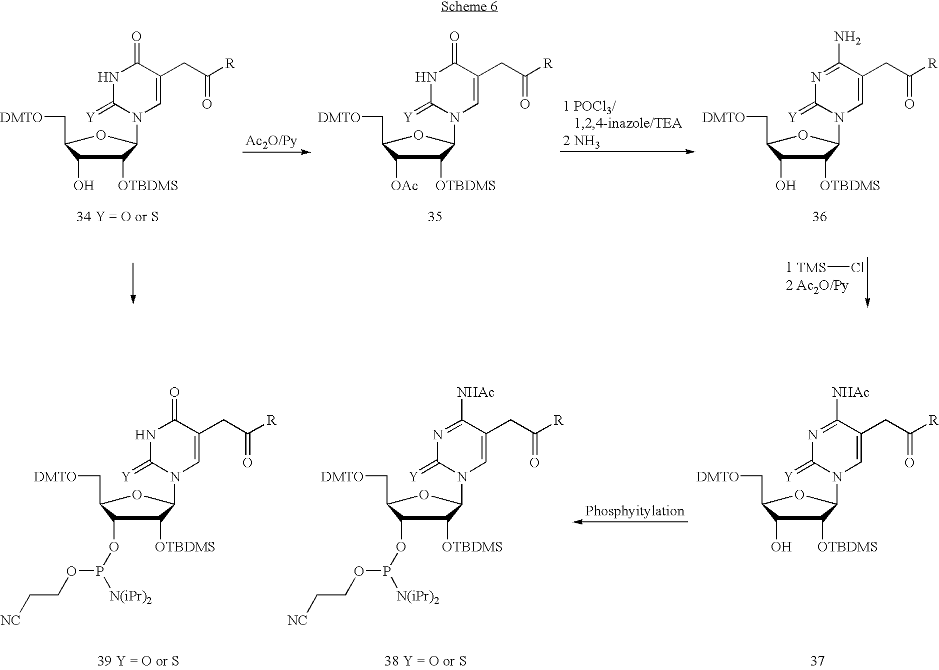

Compound 35 (R=OCH₃, Y=S, Scheme 6): Compound 34 is prepared according to the literature procedure (Bajji and Davis, Org. Lett., 2000, 2, 3865-3868). Treatment of compound 34 with acetic anhydride gives compound 35.

### Example 43

Compound 36 (R=NH₂, Y=S, Scheme 6): Compound 35 in anhydrous acetonitrile is added dropwise into a cold stirring mixture of POCl₃, TEA and 1,2,4-triazole in anhydrous acetonitrile at −20° C. After the addition of compound 35, the reaction mixture is stirred at −20° C. for 3 h. Acetonitrile is removed from the reaction and the residue is extracted with EtOAc, washed with water and bicarbonate solution. After evaporation of EtOAc, the residue is treated with ammonia to obtain compound 36 (Shigeta et. al., Antiviral Chem., 1999, 10, 195-209).

### Example 44

Compound 37 (R=NH₂, Y=S, Scheme 6): The free 3′-hydroxyl group of compound 36 is transiently protected using TMS-Cl and then treated with acetic anhydride in pyridine to obtain compound 37.

### Example 45

Compound 38 (R=NH2, Y=S, Scheme 6): Phosphitylation of compound 37 under the conditions reported in Example 8 yields compound 38.

### Example 46

Compound 38 (R=N1H₂, Y=O, Scheme 6): Phosphoramidite 38 of the cytidine derivative as defined is synthesized from the corresponding cytidine precursor 34 (Y=O) as reported in Examples 42, 43, 44 and 45. Compound 34 is obtained according to literature procedure (Bajji and Davis, Org. Len., 2000, 2, 3865-3868).

### Example 47

Phosphoramidite 39 (R=OMe, Y=O or S, Scheme 6): The phosphoramidite is obtained from compound 34 as reported by Bajji and Davis (Or. Lett., ,2000, 2, 38653868).

### Example 48

Scheme 7 is the synthetic scheme for monomers and intermediates described in Examples 48-53.

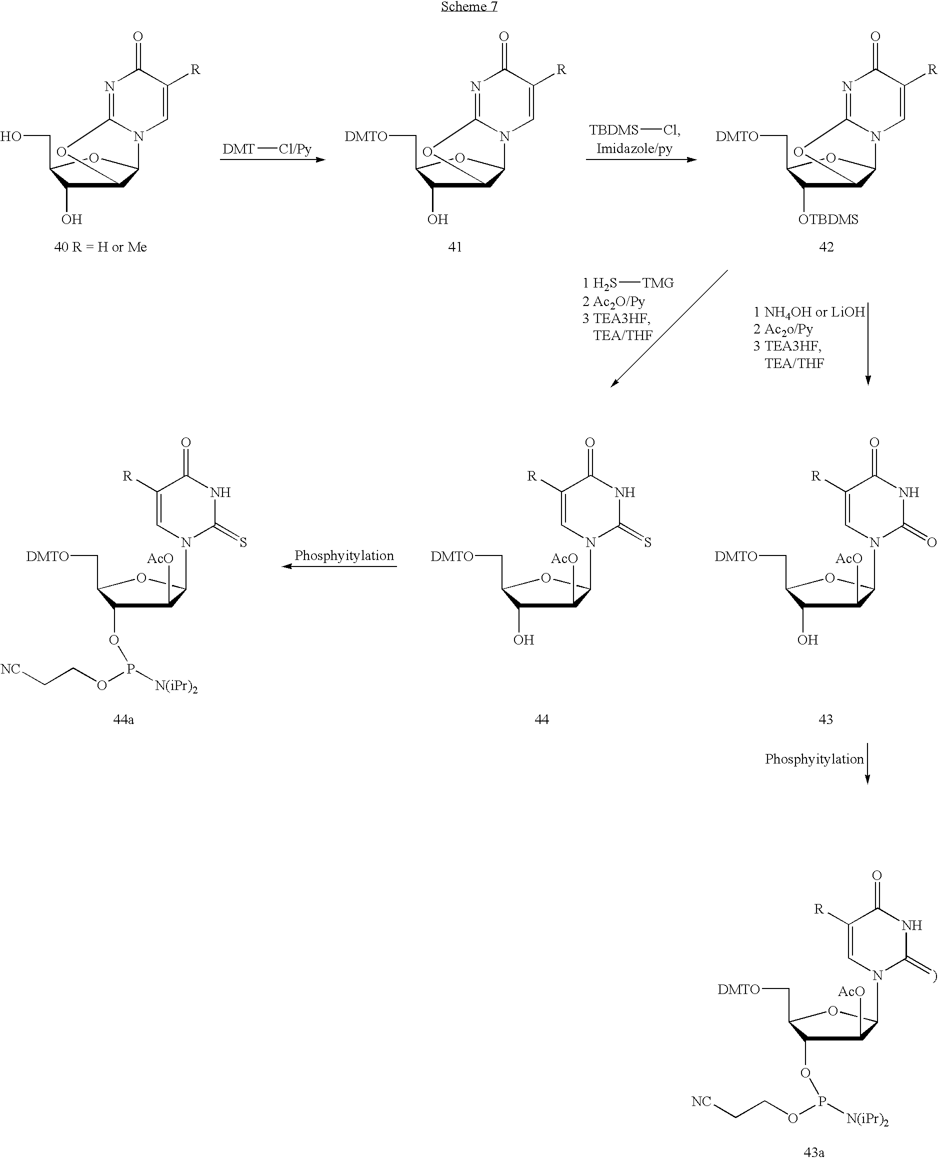

Compound 41 (R=Me, Scheme 7): 2,2′-anhydrouridne 40 is prepared from 5-methyluridine according to the literature procedure (Sebasta et. al., Tetrahedron, 1996, 52, 14385-14402). Reaction of compound 40 with DMT-Cl in the presence of DMAP in pyridine yields compound 41 (McGee et. al., J. Org. Chem., 1996, 61, 781-785).

### Example 49

Compound 42 (R=Me, Scheme 7): Silylation of compound 41 with TBDMS-Cl in the presence of imidazole in pyridine yields compound 42.

### Example 50

Compound 43 (R=Me, Scheme 7): Treatment of the 2,2′-anhydro nucleoside derivative 42 with ammonium hydroxide (Gazz. Chim. Ital., 1990, 120, 661-2) or with LiOH (Collect. Czech. Chem. Commun., 1990, 55, 1801-11) yields the corresponding arabino nucleoside. The arabino nucleoside thus obtained is treated with acetic anhydride in pyridine and subsequent treatment with triethylamine trihydrogenfluoride yields compound 43.

### Example 51

Compound 43a (R=Me, Scheme 7): Phsophitylation of compound 43 as reported in Example 8 yields the phosphoramidate 43a.

### Example 52

Compound 44 (R=Me, Scheme 7): Treatment of compound 42 with hydrogen sulfide in the presence of TMG in pyridine yields the 2-mercapto arabino nucleoside (Jpn. Kokai Tokkyo Koho, 093019931, 25 Nov 1997, Heisei). The arabino nucleoside thus obtained is treated with acetic anhydride in pyridine and subsequent treatment with triethylamine trihydrogenfluoride yields compound 44.

### Example 53

Compound 43a (R=Me, Example 7): Phsophitylation of compound 44 as reported in Example 8 yields the phosphoramidate 44a.

### Example 54

Scheme 8 is the synthetic scheme for monomers and intermediates described in Examples 54-62.

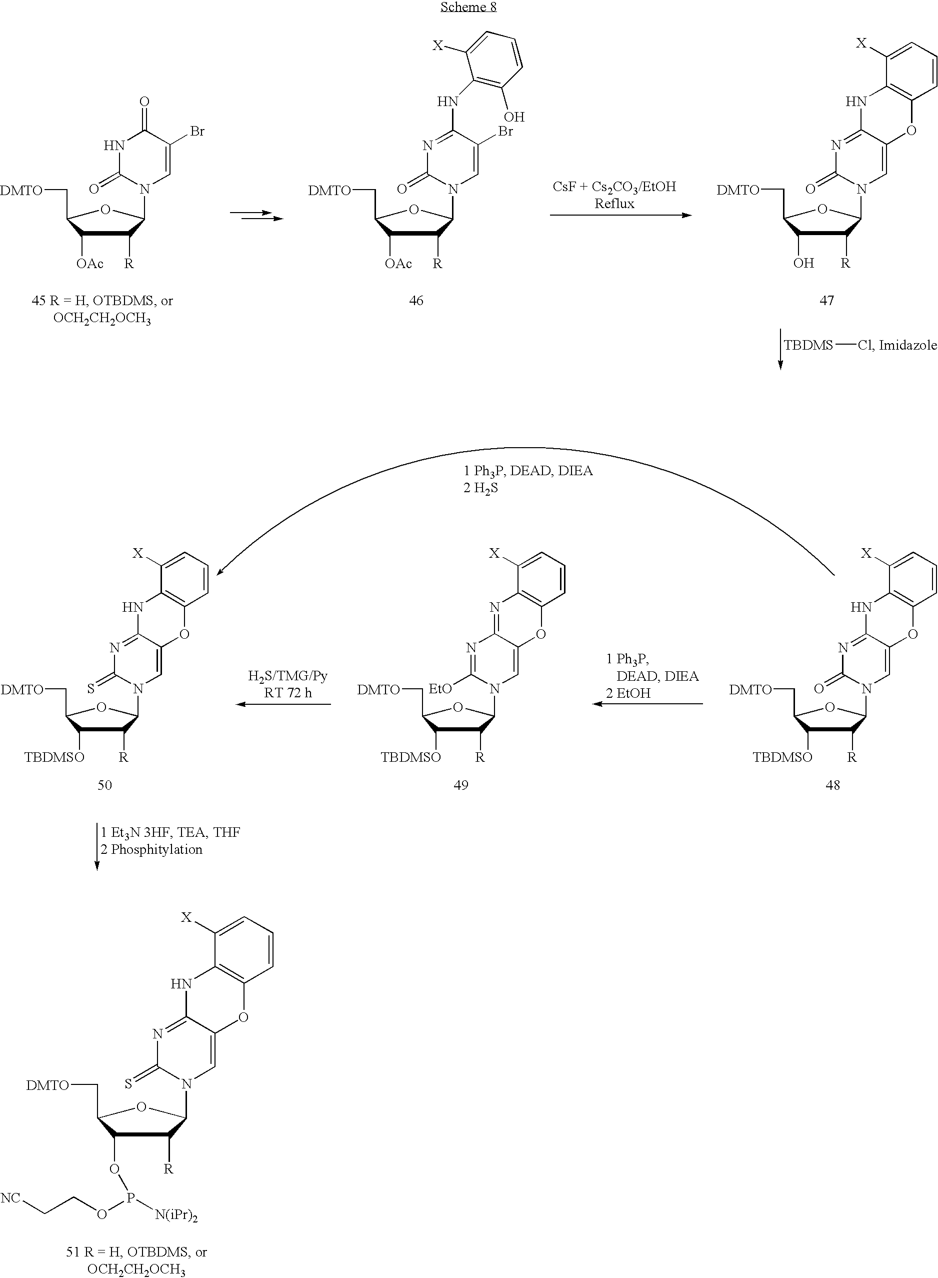

Compound 46 (Scheme 8). Cytidine derivative 46 with desired combination of R(H or OTBDMS or O(CH₂)₂OCH₃) and X (H or O-alkylamino) is synthesized from the corresponding 5-bromo-3′-O-Ac-5′-O-DMT-dU (45) according to the literature procedure by Lin and Matteucci (J. Am. Chem. Soc., 1998, 120, 8531-8532).

### Example 55

Compound 47 (R=H, X=H, Scheme 8). Compound 46 after thorough drying over P₂O₅ is refluxed in absolute ethanol in the presence of 10 molar excess of CsF and 2 molar excess of Cs₂CO₃ to obtain compound 47.

### Example 56

Compound 48 (R=H, X=H, Scheme 8). Silylation of compound 47 with TBDMS-CL as reported in Example 15 yields compound 48.

### Example 57

Compound 49 (R=H, X=H, Scheme 8). Reaction of compound 48 (1 mmol) with ethanol (1 mmol) under Mitsunobu alkylation condition (Ph₃P and DEAD 1 mmol each) in presence of DIEA in acetonitrile yields compound 49.

### Example 58

Compound 50 (R=H, X=H, Scheme 8). Compound 49 (1 mmol) after thorough drying over P₂O₅ under vacuum is taken in a reaction flask under argon. TMG (10 mmol) in anhydrous pyridine, placed on a freezing bath, is saturated with anhydrous H₂S for 45 min. After 45 min, the resulting solution is transferred into the precooled pressure reactor containing compound 49 under argon and is sealed. The sealed vessel is then brought to ambient temperature and is stored at ambient temperature for 3 days. Bubbles off the H2S into a chlorox bath and removes pyridine from the reaction mixture under vacuum. The residue after standard work up and purification yields compound 50.

### Example 59

Compound 50 (R=H, X=H, Scheme 8). Compound 48 is treated with Ph₃P and DEAD in acetonitrile in the presence of DIEA under anhydrous condition and under argon for 1 h. After one hour, anhydrous H₂S gas is passed through the reaction mixture for 10 minute and the mixture is allowed to stir at ambient temperature for overnight to obtain compound 50 in one step from 47.

### Example 60

Compound 51 (R=H, X=H, Scheme 8). Compound 50 is treated with TBAF or triethylamine trihydrofluoride in THF to remove the 3′-O-TBDMS group. The resulting 3′-OH group is subjected to phosphitylation under the conditions described in Example 8 to obtain compound 51.

### Example 61

Compound 51 (R=OTBDMS or O(CH₂)₂OCH₃, X=H, Scheme 8). The ribonucleoside or the 2′-O-MOE phosphoramidite 51 is prepared from the corresponding nucleoside precursor 46 as reported in Examples 56-60.

### Example 62

Compound 51 (R=H or OTBDMS or O(CH₂)₂OCH₃, X=O(CH₂)₂NH₂, Scheme 8). The desired 2-mercapto ‘G-clamp’ (Lin and Matteucci, J. Am. Chem. Soc., 1998, 120, 8531-8532) phosphoramidite 51 is synthesized from the appropriate precursor 46 as reported in Examples 56-60.

### Example 63

Scheme 9 is the synthetic scheme for monomers and intermediates described in Examples 63 and 64.

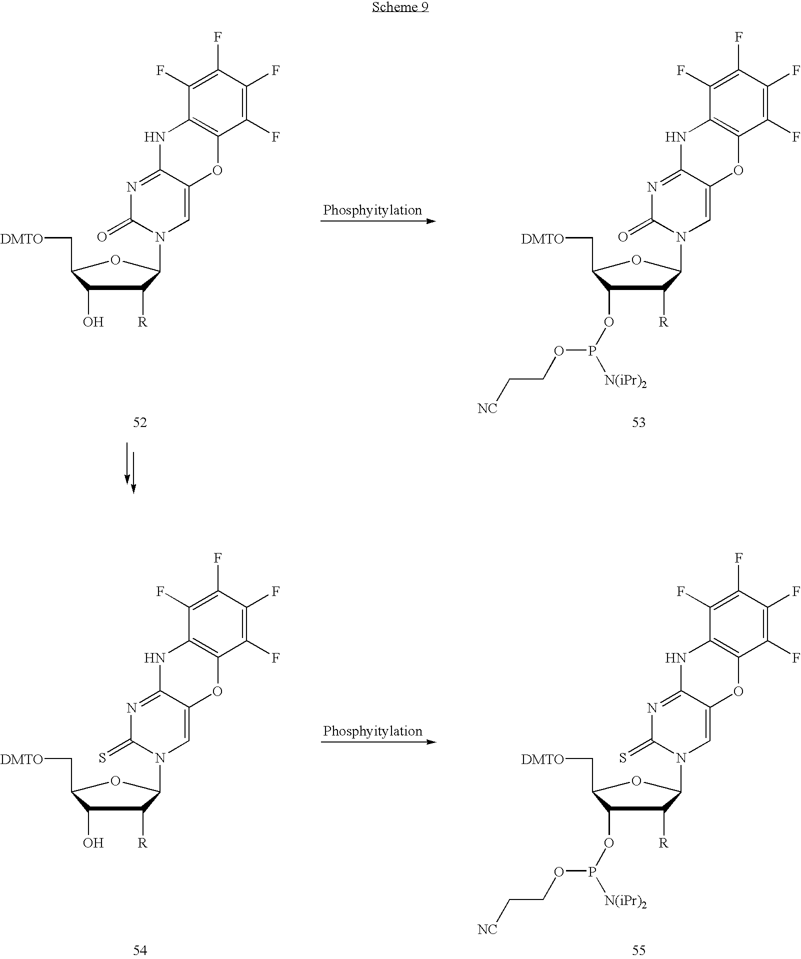

Compound 53 (R=H, X=H, Scheme 9). Compound 52 and the desired phosphoramidite are prepared according to the reported procedure in the literature (Wang et. al., Tetrahedron Lett., 1998, 39, 8385-8388).

### Example 64

Compound 55 (R=H, X=H, Scheme 9). Compound 52 is obtained according to the literature procedure (Wang et. al., Tetrahedron Let., 1998, 39, 8385-8388). The 2-thio analogue 55 of compound 52 is synthesized from compound 52 as reported in Examples 56-60.

### Example 65

Scheme 10 is the synthetic scheme for monomers and intermediates described in Examples 65-72.

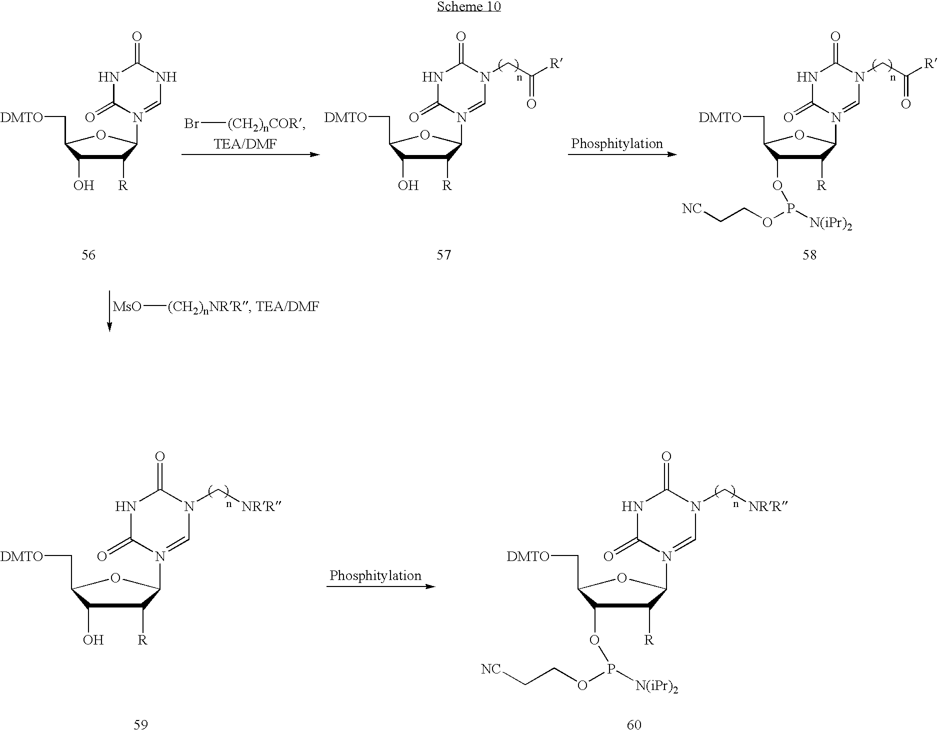

Compound 57 (R=H, R′=OEt, n=1, Scheme 10): Pseudouridine derivative 56 is prepared according to reported procedure (Grohar and Chow, Tetrahedron Lett., 1999, 40, 2049-2052). Compound 56 is stirred with one equivalent of ethylbromoacetate in anhydrous DMF in the presence of triethylamine to obtain compound 57.

### Example 66

Compound 58 (R=H, R′=OEt, n=1, Scheme 10): Phosphitylation of compound 57 under the conditions reported in Example 8 yields the phosphoramidate 58.

### Example 67

Compound 58 (R=H, R′=NH₂, n=1, Scheme 10): Compound 57 upon treatment with ammonia under anhydrous condition yields the corresponding amide, which is then subjected to phosphitylation as reported in Example 8 to obtain compound 58.

### Example 68

Compound 59 (R=H, R′, R″=Me, n=2, Scheme 10). Compound 56 is stirred with [2-(dimethylamino)ethyl]methanesulfonate in the presence of triethylamine in anhydrous DMF to obtain compound 59.

### Example 69

Compound 60 (R=H, R′, R″=Me, n=2, Scheme 10). Phosphitylation of compound 59 as reported in Example 8 yields compound 60.

### Example 70

Compound 58 (R=OTBDMS, R′=OEt, Scheme 10): Compound 56, where R=OTBDMS is prepared according to literature procedure (Gasparotto et. al., Nucleic Acids Res., 1992, 20, 5159-5166). The desired phosphoramidate 58 is obtained from compound 56 by following the procedures reported in Examples 65 and 66.

### Example 71

Compound 58 (R=OTBDMS, R′=NH₂, n=1, Scheme 10): Compound 56, where R=OTBDMS is prepared according to literature procedure (Gasparotto et. al., Nucleic Acids Res., 1992, 20, 5159-5166). The desired phosphoramidate 58 is obtained from compound 56 by following the procedures reported in Examples 65 and 67.

### Example 72

Compound 60 (R=OTBDMS, R′, R″=Me, n=2, Scheme 10): Compound 56, where R=OTBDMS is prepared according to literature procedure (Gasparotto et. al., Nucleic Acids Res., 1992, 20, 5159-5166). The desired phosphoramidate 58 is obtained from compound 56 by following the procedures reported in Examples 68 and 69.

### Example 73

Scheme 11 is the synthetic scheme for monomers and intermediates described in Examples 73-78.

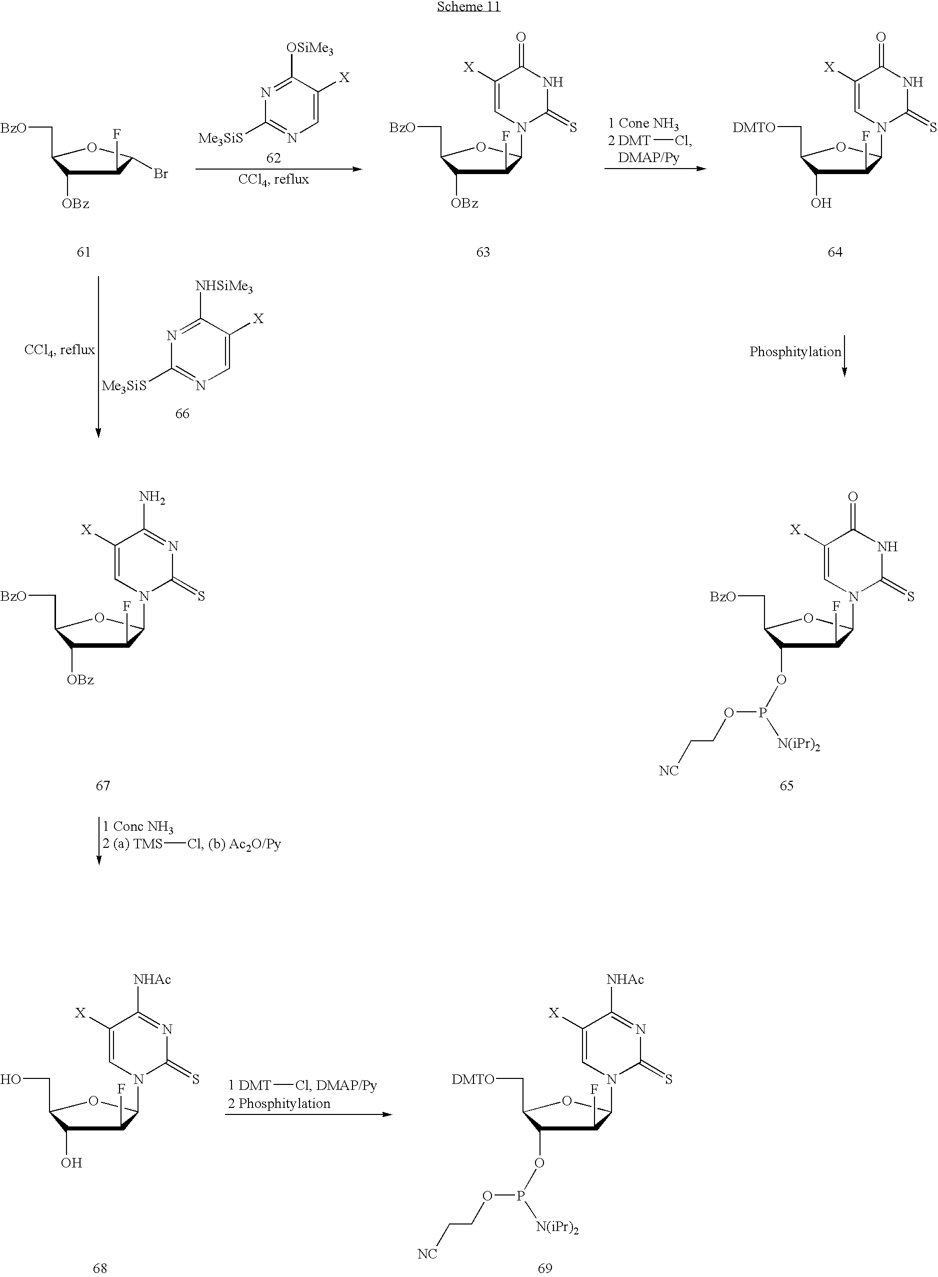

Compound 63 (X=Me, Scheme 11). Compound 61 is obtained from 1,3,5-tri-O-benzoyl-α-D-ribofuranose according to the reported procedure (Wilds and Damha, Nucleic Acids Res., 2000, 28, 3625-3635). A mixture of compound 61 (1 mmol) and 2-S(trimethylsilyl)-4-O-(trimethylsilyl)thymine (62, 1.2 mmol) in CCl₄ is allowed to reflux for 72 h as reported in the literature (Wilds and Damha, Nucleic Acids Res., 2000, 28, 3625-3635). The reaction is quenched with methanol and solid formed is filtered. Evaporation of the solution followed by flash column chromatography yields compound 63.

### Example 74

Compound 64 (X=Me, Scheme 11). Compound 63 is stirred with concentrated ammonia at ambient temperature to deprotect benzoyl groups from 3′ and 5′ hydroxyl groups. This after thorough drying over P₂O₅ is reacted with DMT-Cl in pyridine in the presence of DMAP to obtain compound 64.

### Example 75

Compound 65 (X=Me, Scheme 11). Phosphitylation of compound 64 as reported in Example 8 yields the phosphoramidite 65.

### Example 76

Compound 67 (X=Me, Scheme 11). A mixture of compounds 61 (1 mmol) and 5-methyl-2-S-(trimethylsilyl)-4-N-(trimethylsilyl)cytosine (66, 1.2 mmol) in CCl₄ is allowed to reflux for 72 h. The reaction is quenched with methanol and solid formed is filtered. Evaporation of the solution followed by flash column chromatography yields compound 67 (Wilds and Damha, Nucleic Acids Res., 2000, 28, 3625-3635).

### Example 77

Compound 68 (X=Me, Scheme 11). Compound 67 is stirred with concentrated aqueous ammonia to remove the benzoate. The product thus obtained is transiently protected with trimethylsilyl chloride in anhydrous pyridine and subsequently reacted with acetic anhydride to obtain compound 68.

### Example 78

Compound 69 (X=Me, Scheme 11). The phosphoramidite 69 is prepared from compound 68 in two steps as reported in Examples 74 and 75.

### Example 79

Scheme 12 is the synthetic scheme for monomers and intermediates described in Examples 79-82.

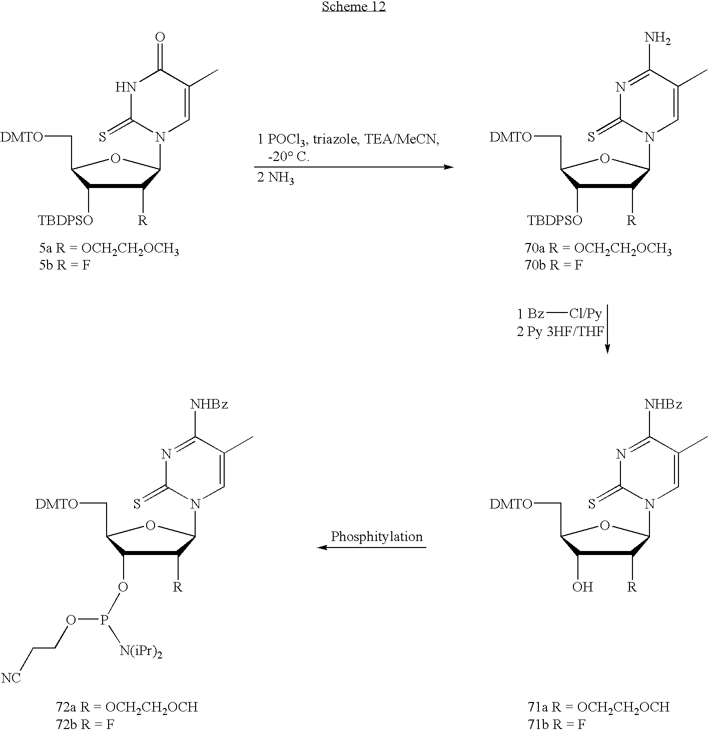

Compound 70a (R=OCH₂CH₂OCH₃, Scheme 12): Compound 5 (1.75 g, 3.07 mmol, obtained from Example 5, Example 1) was treated with DMT-Cl (1.35 g, 3.98 mmol) in the presence of DMAP (50 mg, 0.41 mmol) in anhydrous pyridine as reported in Example 2, to obtain compound 5a (as specified in Example 5). Compound 5a was purified by flash column chromatography; eluent: Hexane/EtOAc (3:1); yield: 2.6 g, (97.1%). ¹H NMR, o (DMSO-d₆): 7.97 (bs, 1H, exchangeable with D₂O), 7.62-7.59 (m, 2H), 7.47-7.10 (m, 17H), 6.99-6.97 (d, 1H, H1′, J=3.00 Hz), 6.86-6.80 (m, 4H), 4.24-4.10 (m, 2H), 4.063.97 (m, 1H), 3.72 (s, 6H), 3.64-3.60 (t, 1H), 3.31-3.20 (m, 4H), 3.17 (s, 3H), 3.02-2.95 (m, 1H), 1.36 (s, 3H), 0.91 (s, 9H).

Compound 5a (2.35 g, 2.69 mmol) was mixed with triazole (1.9 g, 27.53 mmol) and dried overnight over anhydrous P₂O₅ under vacuum. The mixture was suspended in anhydrous CH₃CN under argon and stirred at −20° C. TEA (3.8 mL, 27.26 mmol) was added into the stirring suspension and the stirring was continued for 20 minutes. While maintaining the bath temperature at −20° C., POCl₃ (0.75 mL, 8.06 mmol) was added into the reaction mixture drop-wise. The addition was completed in 20 min and the mixture was allowed stir at −20° C. for 2 h. Removed CH₃CN from the reaction mixture at low temperature under vacuum and the triazolide formed was extracted into ethylacetate, washed with water and saturated sodium bicarbonate solution. Evaporation of ethyl acetate gave a yellow solid. The solid thus obtained was dissolved in THF (10 mL), aqueous ammonia (10 mL) was added into the THF solution and stirred at ambient temperature for 40 min. Removed THF and ammonia from the reaction mixture and the residue in EtOAc (30 mL) was washed with water and sodium bicarbonate solution followed by evaporation of solvent to dryness. The cytidine derivative 70a was finally purified to obtain as a pale yellowish white solid by flash column chromatography; eluent: 3% MeOH in dichloromethane; yield: 2.25 g, (95.9%). ¹H NMR, 6 (CDCl₃-d6): 8.29-8.26 (d, 2H), 7.82 (s, 1H), 7.827.18 (m, 22H), 6.82-6.73 (m, 5H), 4.32-4.27 (m, 2H), 4.09-4.00 (m, 1H), 3.79 (bs, 7H), 3.55-3.35 (m, 4H), 3.30 (s, 3H), 3.10-3.06 (m, 1H), 1.42 (s, 3H), 0.99 (s, 9H).

### Example 80

Compound 71a (Scheme 12): Compound 70a (1.9 g, 2.18 mmol) was dissolved into a mixture of pyridine-dichloromethane (1:1, 10 mL) and stirred at −20° C. under argon. Benzoyl chloride (0.4 mL, 3.45 mmol) was added drop-wise into the stirring solution. The stirring was continued at −20° C. bath temperature for 1 h. Methanol was added into the reaction to quench excess benzoyl chloride. Removed pyridine and dichloromethane in vacuo. The residue was taken in EtOAc (30 mL) and washed with sodium bicarbonate solution followed by standard workup. The N⁴-benzoylated product 70a was purified by flash column chromatography; eluent: 20% EtOAc in Heaxane; yield: 1.41 g (66.4%, yellowish white solid). ¹H NMR, δ (CDCl₃-d₆): 8.29-8.26 (d, 2H), 7.82 (s, 1H), 7.827.18 (m, 22H), 6.82-6.73 (m, 5H), 4.32-4.27 (m, 2H), 4.09-4.00 (m, 1H), 3.79 (bs, 7H), 3.553.35 (m, 4H), 3.30 (s, 3H), 3.10-3.06 (m, 1H), 1.42 (s, 3H), 0.99 (s, 9H).

The compound thus obtained (1.34 g, 1.38 mmol) was dissolved in anhydrous THF (10 mL) under argon and stirred at ambient temperature. To the stirring solution TEA (0.45 mL, 3.23 mmol) was added followed by triethylamine trihydrofluoride (0.85 mL, 5.21 mmol). The reaction mixture was allowed to stir overnight under argon. TBF was removed from the reaction mixture and the residue taken in EtOAc (30 mL) was washed with saturated sodium bicarbonate (20 mL) and water (10 mL). Organic phase was evaporated to a solid mass. The desired N⁴-benzoylated product 71a was finally purified by flash silica gel column chromatography; eluent: 40% EtOAc in hexane; yield: 900 mg (88.9%, yellowish white solid.: ¹H NMR, δ (CDCl₃-d₆+D₂O): 8.30-8.27 (d, 2H), 8.13 (s, 1H), 7.53-7.26 (m, 12H), 6.88-6.84 (m, 4H), 6.49 (s, 1H), 4.53-4.46 (m, 1H), 4.32-4.26 (bm, 1H), 4.17-4.10 (m, 2H), 3.98-3.89 (bm, 1H), 3.80 (s, 6H), 3.65-3.47 (m, 4H), 3.40-3.39 (m, 3H), 1.46 (s, 3H). ¹³C NMR, 8 (CDCl₃): 179.5, 170.9, 158.8, 158.7, 156.1, 144.3, 136.9, 135.3, 135.2, 132.5, 130.2, 129.9, 128.2, 128.1, 128.0, 117.3, 113.3, 93.3, 86.9, 83.6, 83.4, 71.7, 71.6, 68.6, 61.2, 58.9, 55.3, 13.0.

### Example 81

Compound 72a (Scheme 12): Treatment of compound 71a (850 mg, 1.15 mmol) with 2-cyanoethyl tetraisopropylphosphorodiamidite (750 μL, 2.36 mmol) and tetrazole diisopropylammonium salt (200 mg, 1.17 mmol) as reported in Example 8 to obtain compound 72a. The amidite thus formed was purified by flash silica gel column chromatography; eluent: 20% EtOAc in Hexane; yield: 790 mg (73.1%) ³¹P NMR, 8 (CDCl₃-d6): 151.71, 150.74

### Example 82

Compound 72b (R=F, Scheme 12): Compound 5, where R=F, R′=H and R″=DMT, obtained from Example 11 is silylated with TBDMS-Cl in the presence of imidazole in anhydrous pyridine to obtain compound 5b. The desired phosphoramidate 72b is prepared from compound Sb as reported in Examples 79 (appropriate parts of the experimental procedure), 80 and 81.

### Example 83

Scheme 13 is the synthetic scheme for monomers and intermediates described in Examples 83-90.

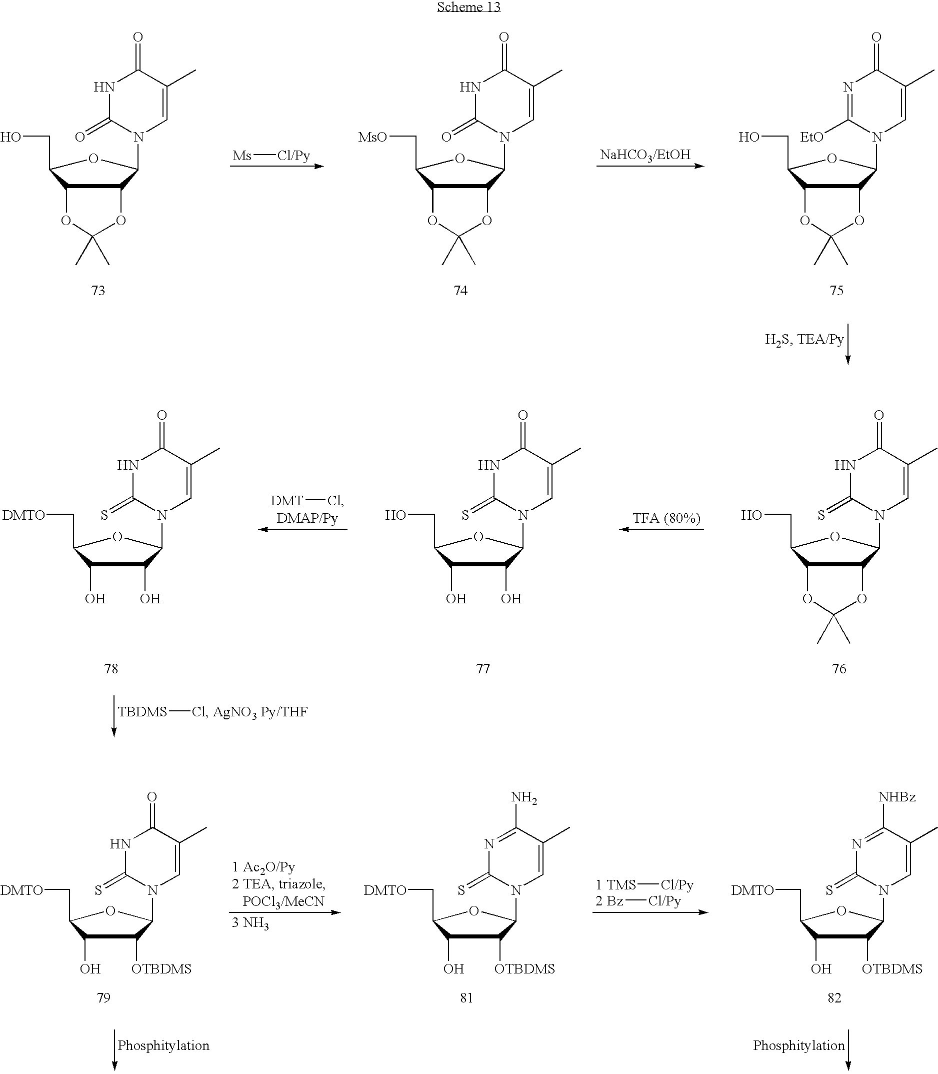

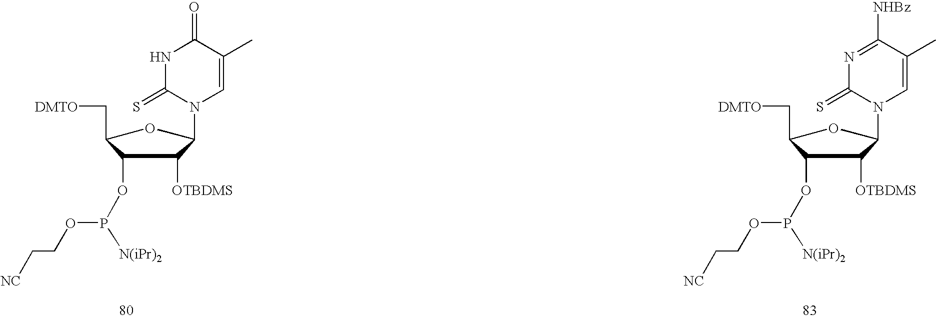

Compound 74 (Scheme 13): Compound 73 was prepared according to the literature procedure (Kumar and Walker, Tetrahedron, 1990, 46, 3101-10). Compound 73 (42.5 g, 142.62 mmol) was dissolved in pyridine-dichloromethane (1:1, 150 mL) and stirred at −20° C. under argon. Methanesulfonyl chloride (22 mL, 284.24 mmol) was added drop-wise into the stirring solution, the addition was completed in 10 min and the mixture was allowed to stir for 1 h at −20° C. Removed pyridine and dichloromethane in vacuo and the residue suspended in EtOAc (400 mL) was washed with water and saturated sodium bicarbonate solution. After removal of the ethyl acetate in vacuo, the residue was redissolved in dichloromethane (200 mL) and treated with activated charcoal, filtered through a column of celite and evaporated to a white solid, yield: 52.16 g (97.3%). ¹H NMR (200 MHz, DMSO-d₆): δ 11.40 (s, 1H, exchangeable with D₂O), 7.55 (s, 1H), 5.81 (s, 1H), 5.06-5.03 (m, 1H), 4.84-4.79 (m, 1H), 4.43-4.23 (m, 3H), 3.19 (s, 3H), 1.76 (s, 3H), 1.49 (s, 3H), 1.29 (s, 3H). ¹³C NMR (50 MHz, DMSO-d₆): δ 163.8, 150.3, 138.4, 113.5, 109.7, 92.2, 83.8, 83.3, 80.4, 69.4, 36.7, 26.9, 25.1, 11.9.

### Example 84

Compound 75 (Scheme 13): Compound 74 (47.5 g, 126.33 mmol) and NaHCO₃ (21.23 g, 252.71 mmol) were mixed in a 200 ML RB and dried over P₂O₅ under vacuum overnight. Absolute ethanol (200 proof, 200 mL) was added into the mixture under argon atmosphere and refluxed for 48 h under argon. The reaction mixture was cooled to room temperature and filtered through a sintered funnel, the solid residue was thoroughly washed with methanol, combined the washing and concentrated to 50 mL. Compound 75 was precipitated from the solution by adding diethyl ether (200 mL) in to the methanolic solution. The precipitate was filtered and dried over P₂O₅ under vacuum overnight to obtain a white solid, 28.54 g (69.3%). ¹H NMR (200 MHz, DMSO-d₆): δ 7.71 (s, 1H), 5.81-5.80 (d, 1H, H1′, J=2.60 Hz), 5.16 (s, 1H, exchangeable with D₂O), 4.92-4.87 (m, 1H), 4.77-4.72 (m, 1H), 4.39-4.28 (q, 2H), 4.11-4.09 (bm, 1H), 3.59 (bs, 2H), 1.78 (s, 3H), 1.49 (s, 3H), 1.33-1.26 (m, 6H). ¹³C NMR (50 MHz, DMSO-d₆): δ 170.8, 154.8, 135.2, 116.0, 113.4, 92.0, 86.3, 84.2, 80.3, 64.7, 61.2, 27.2, 25.4, 14.1, 13.5.

### Example 85

Compound 76 (Scheme 13): Compound 75 (6.15 g, 18.87 mmol) was dried over P₂O₅ under vacuum overnight and was treated with H₂S and triethylamine in anhydrous pyridine as reported in Example 3. After removing H₂S and pyridine the product was precipitated out from water, filtered, washed with water and diethyl ether to obtain the desired compound 76 as a white solid, 5.49 g (92.7%). ¹H NMR (200 MHz, DMSO-d₆): δ12.65 (s, 1H, exchangeable with D₂O), 7.88 (s, 1H), 6.90-6.89 (d, 1H, H1′, J=1.40 Hz), 5.34-5.29 (t, 1H, exchangeable with D₂O), 4.80 (bm, 2H), 4.10-4.09 (m, 1H), 3.69-3.62 (m, 2H), 1.81 (s, 3H), 1.50 (s, 3H), 1.28 (s, 3H). ¹³C NMR, (50 MHz, DMSO-d₆): δ 175.2, 160.7, 137.4, 115.8, 113.6, 92.7, 86.1, 84.4, 79.5, 27.3, 25.5, 12.6.

### Example 86

Compound 77 (Scheme 13): Compound 76 (5.1 g, 16.24 mmol) was stirred in 80% trifluoroacetic acid (60 mL) for 6 h. After removing the acid and water from the reaction, the residue was thoroughly washed with ethyl acetate followed by drying under vacuum over P₂O₅ to obtain compound 77 as a white solid, yield 3.85 g (86.5%). ¹H NMR (200 MHz, DMSO-d₆): δ 12.55 (s, 1H, exchangeable with D₂O), 8.11-8.10 (d, 1H, H6 J=1.20 Hz), 6.55-6.53 (d, 1H, H1′, J=3.40 Hz), 4.06-3.55 (m, 5H), 1.80-1.79 (d, 3H). ³C NMR, (50 MHz, DMSO-d₆): δ 175.1, 160.6, 137.1, 114.8, 92.5, 84.5, 74.4, 68.8, 59.8, 12.5.

### Example 87

Compound 78 (Scheme 13): Compound 77 (3.5 g, 12.77 mmol) was treated with DMTCl (4.76 g, 14.05 mmol) in the presence on DMAP (350 mg, 2.86 mmol) in anhydrous pyridine as reported in Example 2 to obtain the desired compound. The compound 78 was purified by flash silica gel column chromatography; eluent: 4% methanol in dichloromethane; yield: 4.37 g, 59.4 g. ¹H NMR (200 MHz, CDCl₃): δ 7.93-7.92 (d, 1H, J=1 Hz), 7.42-7.19 (m, 9H), 6.87-6.81 (m, 4H), 6.48-6.47 (d, 11H, H1′, J=2 Hz), 4.49-4.41 (m, 2H), 4.26-4.23 (m, 1H), 3.79 (s, 6H), 3.63-3.40 (m, 2H), 1.45-1.44 (d, 3H, J=0.4 Hz).

### Example 88

Compound 79 (Scheme 13): Compound 79 is obtained from compound 78 and TBDMSCl as reported in Example 37.

### Example 89

Compound 80 (Scheme 13): Phosphitylation of compound 79 as reported in Example 8 yields the desired phosphoramidate 80.

### Example 90

Compound 83 (Scheme 13): Compound 83 is obtained from compound 79 as reported in Examples 79 (appropriate parts of experimental procedure), 80 and 81.

### Example 91

Scheme 14a is the synthetic scheme for monomers and intermediates described in Examples 91-104.

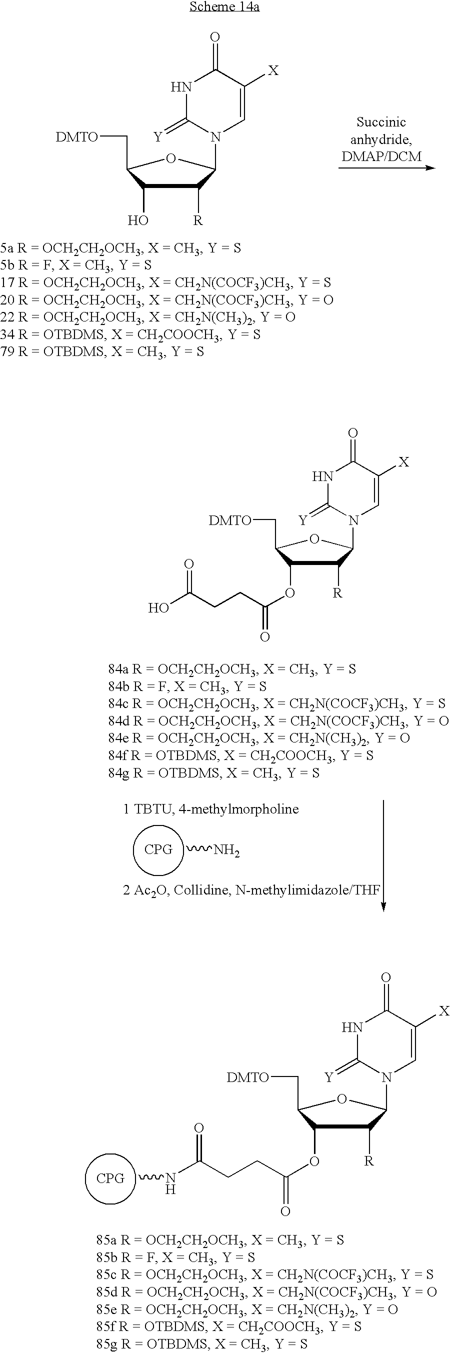

Compound 84a (R=OCH₂CH₂OCH₃, Scheme 14a): Compound 5a (1 mmol) is mixed with succinic anhydride (2 mmol) and dimethlyaminopyridine (1 mmol), and is dried over P₂O₅ in vacuo overnight. Dichloromethne (0.9 mL) is added into the mixture and stirs at ambient temperature for 8 h. The reaction mixture is diluted with excess dichloromethane and the organic layer is subjected ice cold aqueous citric acid wash (10% solution) and brine. The organic phase is dried over anhydrous Na₂SO₄ and concentrated to dryness to yield the succinic acid derivative 84a.

### Example 92

Compound 85a (R=OCH₂CH₂OCH₃, Scheme 14a): Compound 84a (1 mmol) is dried over P₂O₅ under vacuum overnight. Anhydrous DMF is added into the dried 84a and mixed with 2-(1H-benzotriazole-1-yl)-1,1,3,3-tetramethyluronium tetrafluoroborate (TBTU, 1 mmol) and 4-methylmorpholine (2 mmol) with vortexing to give a clear solution. Calculated amount of CPG (118.9 μmol/g, particle size 80/120, mean pore diameter 569 Å) is added into the clear solution and allows to shake on a shaker at ambient temperature for 18 h. An aliquot of the support is withdrawn and washed with DMF, CH₃CN and diethylether, and dries in vacuo. Loading capacity is determined by following standard procedure. Functionalized CPG is then washed with DMF, CH₃CN, diethylether and dried in vacuo. Unfunctionalized sites on the CPG are capped with acetic anhydride/collidine/N-methylimidazole in THF (2 mL Cap A and 2 mL Cap B solutions from Perspective Biosystems Inc.) and allows to shake on a shaker for 2 h. The CPG is filtered, washed with CH₃CN followed by diethlether, and dries in vacuo. The final loading capacity of 85a is determined after capping.

### Example 93

Compound 85b (Scheme 14a): The desired solid support 85b is obtained from its corresponding precursor 5b as reported in Examples 91 and 92.

### Example 94

Compound 85c (Scheme 14a): The desired solid support 85c is obtained from its corresponding precursor 17 as reported in Examples 91 and 92.

### Example 95

Compound 85d (Scheme 14a): The desired solid support 85d is obtained from its corresponding precursor 20 as described in Examples 91 and 92.

### Example 96

Compound 85e (Scheme 14a): The desired solid support 85e is obtained from its corresponding precursor 22 as described in Examples 91 and 92.

### Example 97

Compound 85 g (Scheme 14a): The desired solid support 85f is obtained from its corresponding precursor 34 as described in Examples 91 and 92.

### Example 98

Compound 85f (Scheme 14a): The desired solid support 85f is obtained from its corresponding precursor 79 as described in Examples 91 and 92.

### Example 99

Scheme 14b is the synthetic scheme for monomers and intermediates described in Examples 99-104.

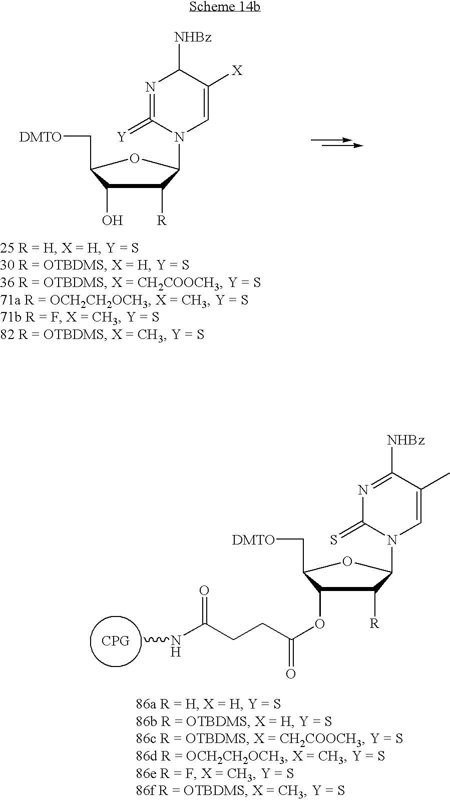

Compound 86a (Scheme 14b): The desired solid support 86a is obtained from its corresponding precursor 25 as described in Examples 91 and 92.

### Example 100

Compound 86bh (Scheme 14b): The desired solid support 86b is obtained from its corresponding precursor 30 as described in Examples 91 and 92.

### Example 101

Compound 86c (Scheme 14b): The desired solid support 86c is obtained from its corresponding precursor 36 as described in Examples 91 and 92.

### Example 102

Compound 86d (Scheme 14b): The desired solid support 86d is obtained from its corresponding precursor 71a as described in Examples 91 and 92.

### Example 103

Compound 86e (Scheme 14b): The desired solid support 86e is obtained from its corresponding precursor 71b as described in Examples 91 and 92.

### Example 104

Compound 86f (Scheme 14b): The desired solid support 86f is obtained from its corresponding precursor 82 as described in Examples 91 and 92.

### Example 105

Scheme 14c is the synthetic scheme for monomers and intermediates described in Examples 105-107.

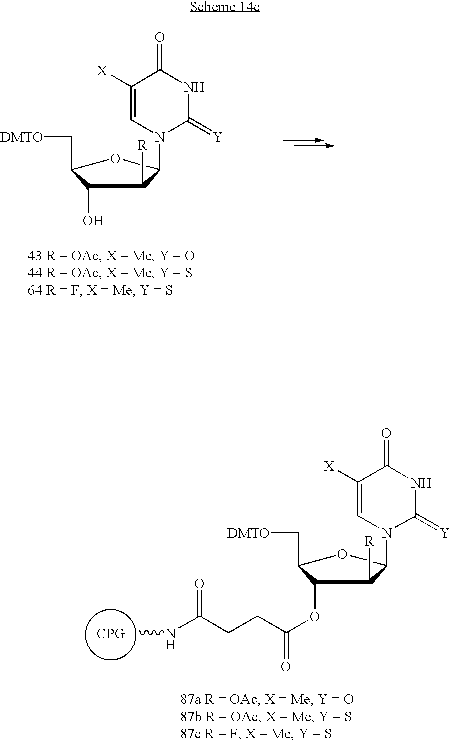

Compound 87a (Scheme 14c): The desired solid support 87a is obtained from its corresponding precursor 43 as described in Examples 91 and 92.

### Example 106

Compound 87b (Scheme 14c): The desired solid support 87b is obtained from its corresponding precursor 44 as described in Examples 91 and 92.

### Example 107

Compound 87c (Scheme 14c): The desired solid support 87c is obtained from its corresponding precursor 64 as described in Examples 91 and 92.

### Example 108

Scheme 15 is the synthetic scheme for monomers and intermediates described in Examples 108-119 and 121-124.

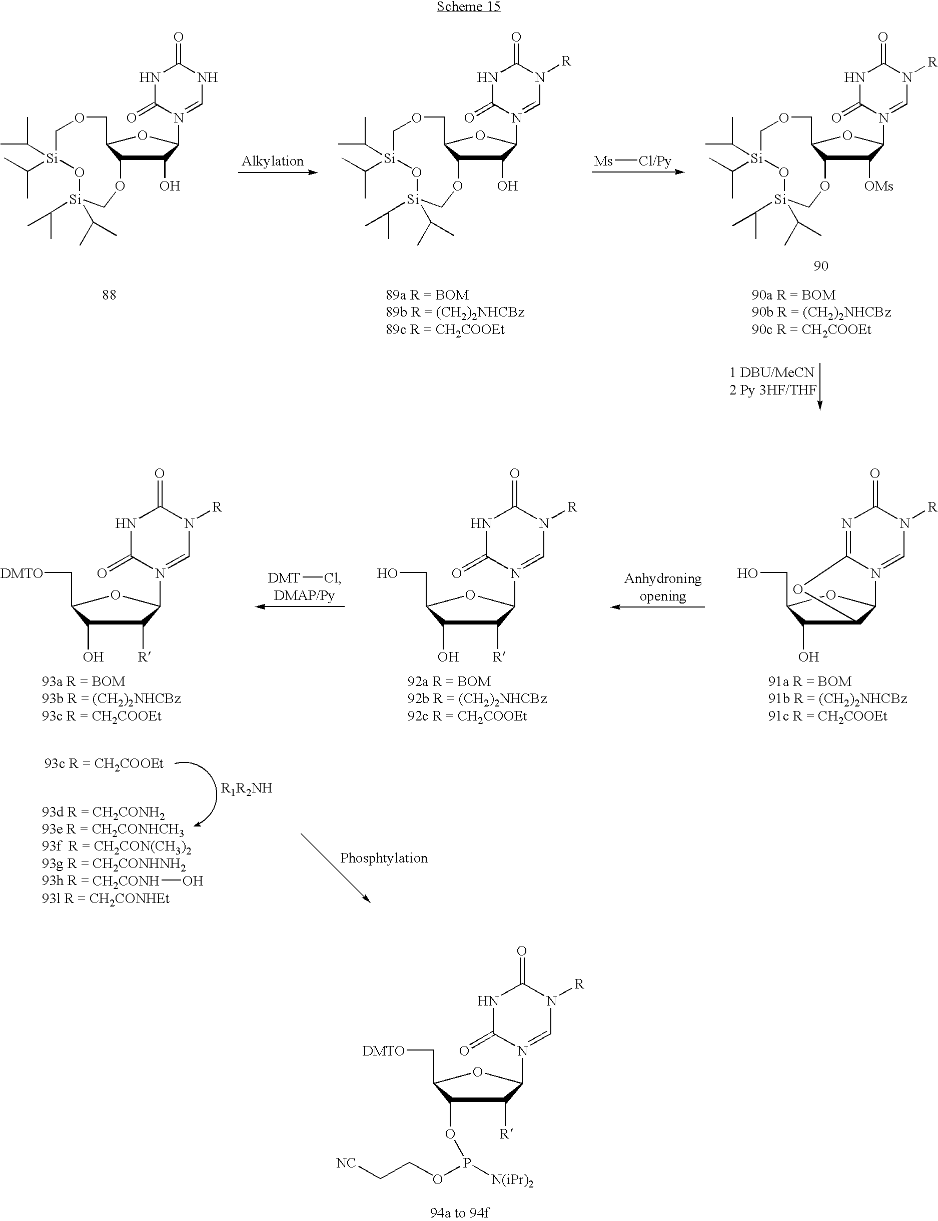

Compound 89a (R=BOM, Scheme 15): Compound 88 is prepared according to the literature procedure (Nucleosides Nucleotides, 1985, 4, 613-24). Compound 88 (1 mmol) is stirred with BOM-CI in dichloromethane in the presence of TEA to obtain compound 89a.

### Example 109

Compound 90a (R=BOM, Scheme 15): Compound 89a is stirred in pyridine with methanesulfonyl chloride at 0° C. for 1 hr to obtain compound 90a.

### Example 110

Compound 91a (R=BOM, Scheme 15): Compound 90a is treated with DBU in MeCN to obtain the corresponding sugar protected anhydro derivative. Treatment of the protected nucleoside thus obtained with pyridinium trihydrogen fluoride in anhydrous THF yields compound 91a.

### Example 111

Compound 92a (R=BOM, R′=OCH₂CH₂OCH₃, Scheme 15): The compound 91a (1 mmol) with 2 equivalent of (CH₃OCH₂CH₂O)₃B in the presence of PTSA yields 2′-O-metohxyethyl-pseudouriidne 92a.

### Example 112

Compound 93a (R=BOM, R′=OCH₂CH₂OCH₃, Scheme 15): Compound 92a is stirred with DMT-Cl in anhydrous pyridine in the presence of DMAP as described in Example 2 to obtain compound 93a.

### Example 113

Compound 94a (R=H R′=OCH₂CH₂OCH₃, Scheme 15): Catalytic reduction of compound 93a followed by basic hydrolysis gives the corresponding Ni deprotected nucleoside (Macor et. al., Tetrahedron Let., 1977, 38, 1673). Phosphitylation of compound, obtained from the reductive hydrolysis, as described in Example 8 yields compound 94a.

### Example 114

Compound 89b (R=CH₂CH₂NHCbz, Scheme 15): Compound 88 is stirred with Ncarbobenzyloxyethanolamine-O-mesylate [(CBz)HNCH₂CH₂OSO₂Me] in the presence of base to obtain compound 89b. The mesylate is prepared from Ncarbobenzyloxyethanolamine according to standard procedure.

### Example 115

Compound 92b (R=CH₂CH₂NHCbz, R′=OCH₂CH₂OCH₃, Scheme 15): Compound 92b as defined is obtained from compound 89b as described in Examples 109, 110 and 111.

### Example 116

Compound 93b (R=(CH₂)₂NHCOCF₃, R′=OCH₂CH₂OCH₃, Scheme 15): 5′-hydroxyl function of compound 92b is protected as its DMT derivative as described in Example 2. Compound thus obtained is treated with 10 molar excess of ammonium formate in the presence of 10% activated Pd-C in EtOAc for 10 min. The side chain free amino group thus formed is stirred with ethyltrifluoroacete in the presence of TEA in dichloromethane to obtain compound 93b.

### Example 117

Compound 94b (R=(CH₂)₂NHCOCF₃, R′=OCH₂CH₂OCH₃, Scheme 15): Phosphitylation of compound 93b with 2-Cyanoethyl tetraisopropylphosphrodiamidite as reported in Example 8 yields compound 94b.

### Example 118

Compound 89c (R=CH₂CO₂Et, Scheme 15): Compound 88 is stirred with ethylbromoacetate in the presence of DIEA in DCM to obtain compound 89c.

### Example 119

Compound 93c (R=CH₂CO₂Et, R′=OCH2CH₂OCH₃, Scheme 15): Compound 93c as defined is obtained from compound 89c according to the procedure reported in Examples 109 to 112.

### Example 120

Compound 94c (R=CH₂CO₂Et, R′=OCH₂CH₂OCH₃, Scheme 1): Compound 93c from Example 119 is phosphitylated as described in Example 8 to obtain the phosphoramidite 94c.

### Example 121

Compound 93d to 93i (R=CH₂COY, R′=OCH₂CH₂OCH₃, Scheme 15): Compound 93c obtained from Example 119 is treated with:

(a) ammonia to obtain compound 93d (Y=NH₂);

(b) methylamine to obtain compound 93e (Y=NHMe);

(c) dimethylamine to obtain compound 93f (Y=NMe₂);

(d) hydrazine to obtain compound 93 g (Y=NH—NH₂);

(e) hydroxylamine to obtain compound 93 h (Y=NH—OH);

(f) ethylamine to obtain compound 93i (Y=NHEt).

### Example 122

Compound 94d (R=CH₂CONH₂, R′=OCH₂CH₂OCH₃, Scheme 15): Phosphitylation of compound 93d as described in Example 8 yields the phosphoramidate 94d.

### Example 123

Compound 94e (R=CH₂CONHCH₃, R′=OCH₂CH₂OCH₃, Scheme 15): Phosphitylation of compound 93e as described in Example 8 yields the phosphoramidate 94e.

### Example 124

Compound 94f (R=CH₂CON(CH₃)₂, R′=OCH₂CH₂OCH₃, Scheme 15): Phosphitylation of compound 93f as described in Example 8 yields the phosphoramidate 94e.

### Example 125

Scheme 16 is the synthetic scheme for monomers and intermediates described in Example 125.

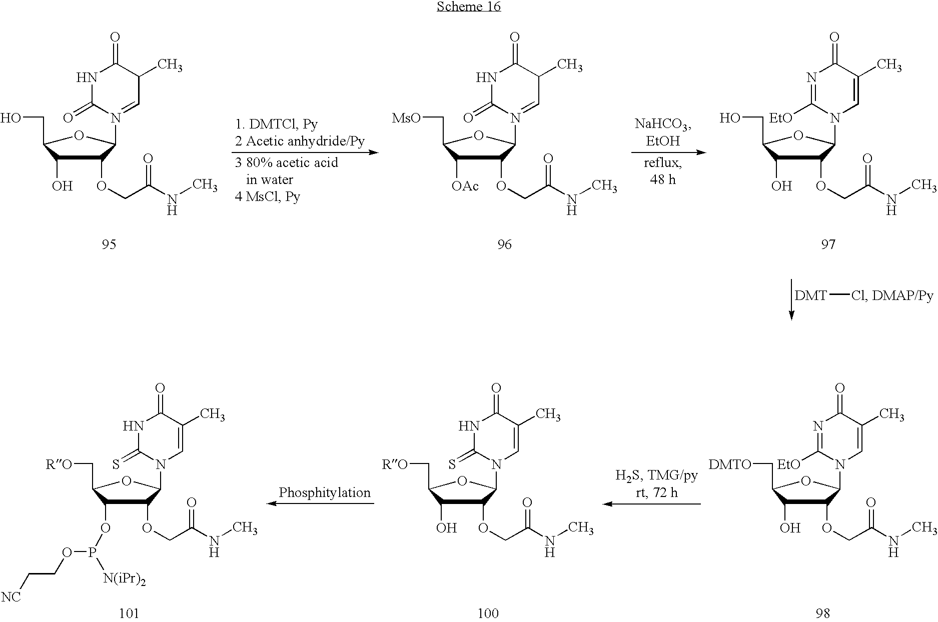

Compound 101 (Scheme 16): Compound 95 is prepared according to the procedure described in the literature (U.S. Pat. No. 6,147,200). Tritylation at 5′-O— position of compound 95 with DMT-Cl in pyridine at room temperature, then acetylation at 3′-O-positon with acetic anhydride in pyridine yields 5′-O-DMT-3′-O-acetyl derivative. Detritylation with 80% acetic acid followed by treatment with methanesulfonyl chloride in pyridine yields compound 96. Compound 101 is prepared from compound 96 according to the procedure described for the synthesis of compound 6 from compound 2 in Example 1.

### Example 126

Scheme 17 is the synthetic scheme for monomers and intermediates described in Example 126.

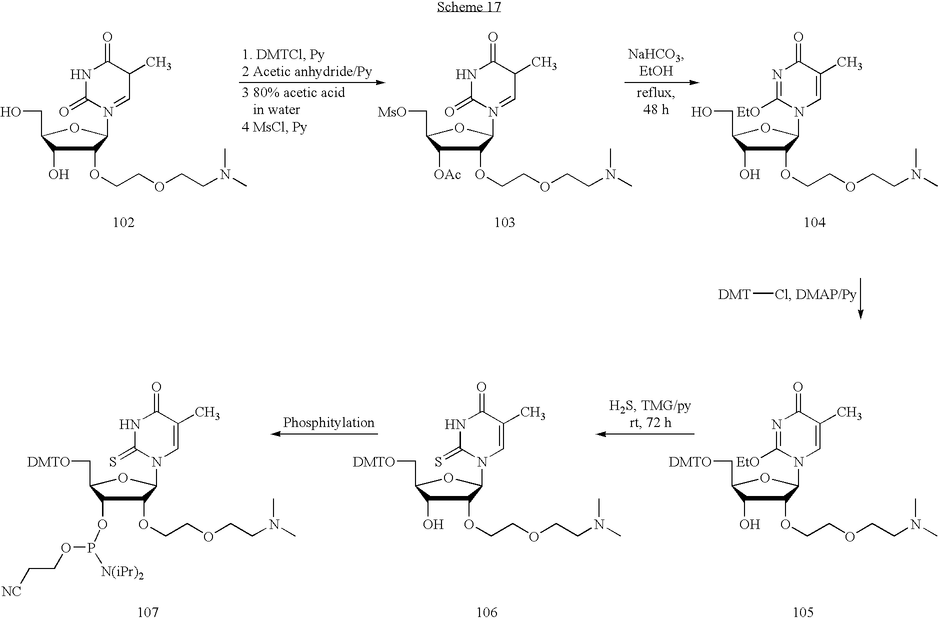

Compound 107 (Scheme 17): Compound 102 is prepared according to the procedure described in the literature (U.S. Pat. No. 6,043,352). Tritylation at 5′-O-position of compound 102 with DMT-Cl in pyridine at room temperaturet, followed by acetylation at 3′-O-positon with acetic anhydride in pyridine yields 5′-O-DMT-3′-O-acetyl derivative. Detritylation with 80% acetic acid followed by treatment with methanesulfonyl chloride in pyridine yields compound 103. Compound 107 is prepared from compound 103 according to the procedure described for the synthesis of compound 6 from compound 2 in Example 1.

### Example 127

Scheme 18 is the synthetic scheme for monomers and intermediates described in Example 127.

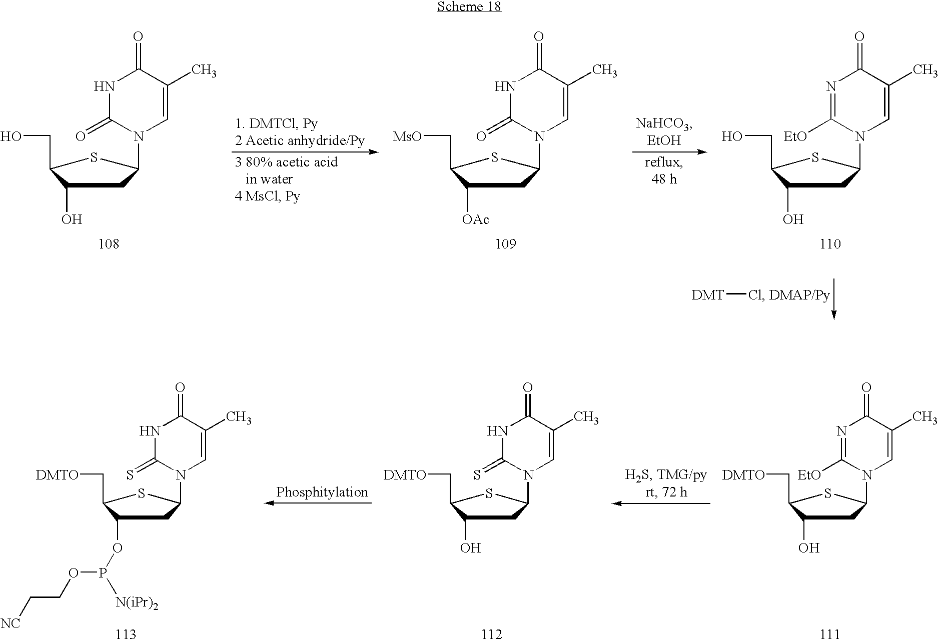

Compound 113 (Scheme 18): Compound 108 is prepared according to the procedure reported (Secrist, J, A. et al. J. Med. Chem. 1991, 56, 2361-2366, Tiwari, K. N. et. al. Nucleosides, Nucleotides 1995, 14, 675-686). Tritylation at 5′-O— position of compound 102 with DMT-Cl in pyridine at room temperature, then acetylation at 3′-O-positon with acetic anhydride in pyridine yields 5′-O-DMT-3′-O-acetyl derivative. Detritylation with 80% acetic acid followed by treatment with methanesulfonyl chloride in pyridine yields compound 109. Compound 113 is prepared from compound 109 according to the procedure described for the synthesis of compound 6 from compound 2 in Example 1.

### Example 128

Scheme 19 is the synthetic scheme for monomers and intermediates described in Example 128.

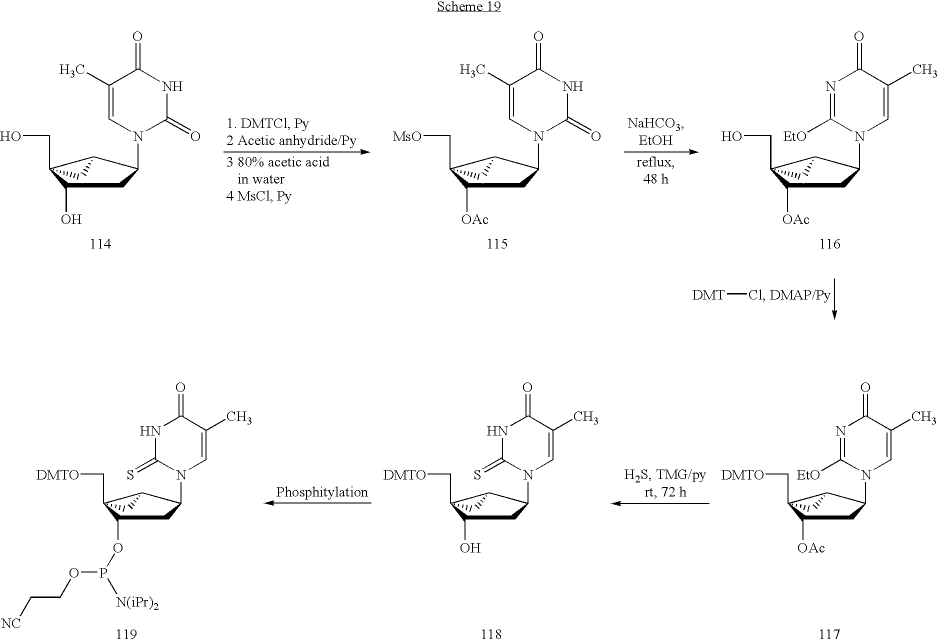

Compound 119 (Scheme 19): Compound 114 is prepared according to the procedure reported (Ezzitouni, A. et. al. J. Org. Chem. 1997, 62, 4870-4873). Tritylation at 5′-Oposition of compound 114 with DMT-Cl in pyridine at rt, then acetylation at 3′-O-positon with acetic anhydride in pyridine yields 5′-O-DMT-3′-O-acetyl derivative. Detritylation with 80% acetic acid followed by treatment with methanesulfonyl chloride in pyridine yield compound 115. Compound 119 is prepared from compound 115 according to the procedure described for the synthesis of compound 6 from compound 2 in Example 1.

### Example 129

Scheme 20 is the synthetic scheme for monomers and intennediates described in Example 129.

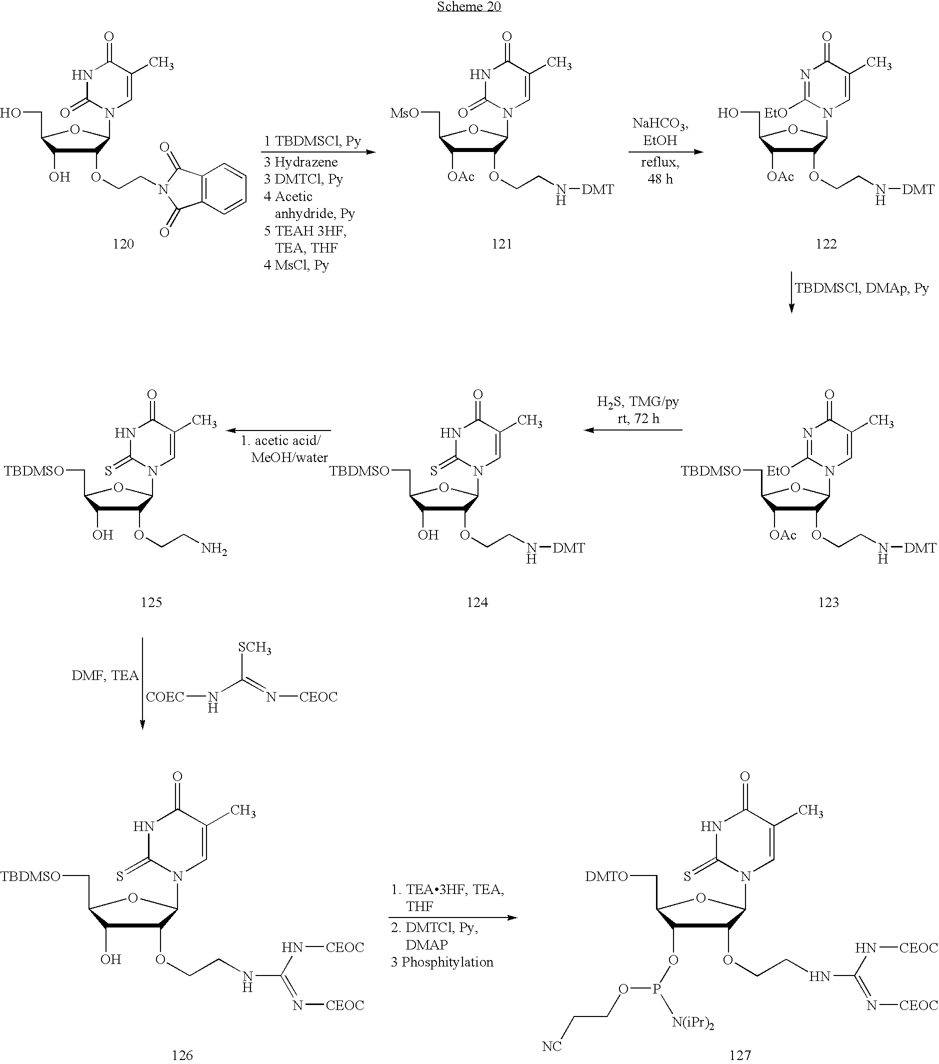

Compound 127 (Scheme 20): Compound 120 is prepared according to the procedure reported (Manoharan M. et. al. J. Org. Chem. 1999, 64, 6468-6472). Silylation of compound 120 with TBDMS-Cl yield 5′-O-TBDMS derivative which on refluxing with hydrazine with methanol give 2′-O-[2-(amino)ethyl derivative, then amino group at 2′ side chain is protected with DMT group by reacting with DMT-Cl in pyridine then acetylation of 3′ hydroxyl group with acetic anhydride in pyridine yield 5′-O-TBDMS-3′-O-acetyl-2′-O-[2-(DMT-amino)ethyl-5-methyl uridine. This is then desilylated with triethylamine trihydofluoride and triethylamine in THF, then treatment with methanesulfonyl chloride in pyridine yields 121. Compound 121 is refluxed in ethanol in presence of NaHCO₃ to yield compound 122, which is subsequently treated with TBDMS-Cl in pyridine to get compound 123. A saturated solution of H₂S in pyridine and tetramethyl guanidine is added to compound 123 and keep at room temperature to get 124. Compound 124 is treated with acetic acid in water to get compound 125. The compound 125 on treatment with N,N′-bis-CEOC-2-methyl-2-thiopseudourea (prepared as reported in U.S. patent application Ser. No. 09/612,531, filed Jul. 7, 2000, the specification of which is incorporated herein by reference) in DMF and TEA at room temperature to yield compound 126. Desilylation of compound 126 with TEA.3HF and TEA in THF, then tritylation at 5′-position followed by phosphitylation at 3′-postion yields compound 127.

### Example 130

Scheme 21 is the synthetic scheme for monomers and intermediates described in Examples 130-132.

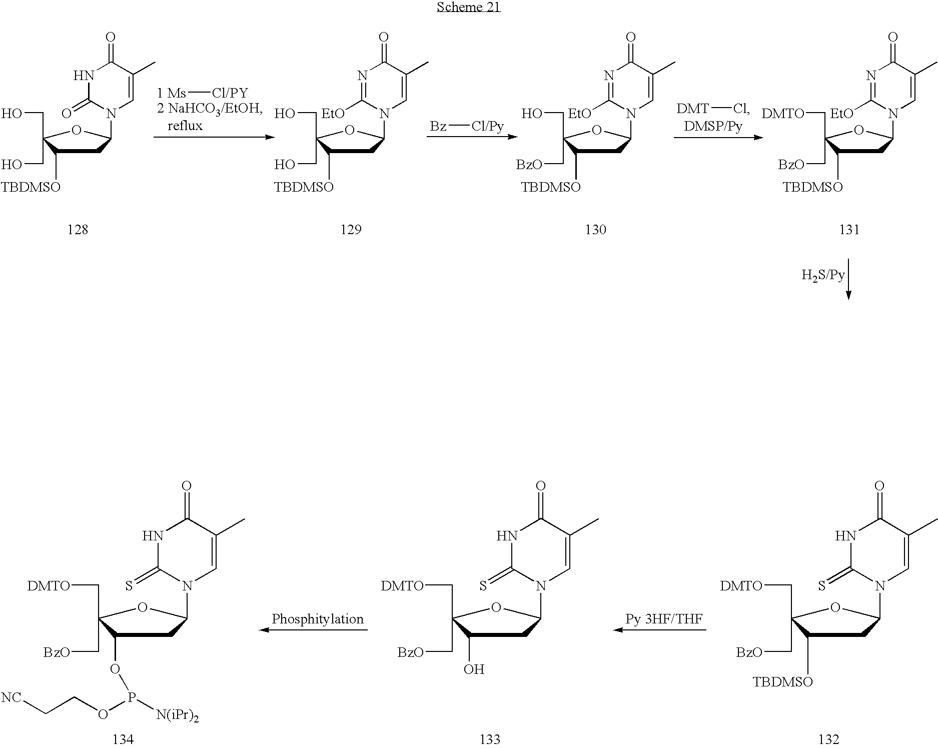

Compound 129 (Scheme 21): Compound 128 is prepared according to the literature procedure (Thrane et. al., Tetrahedron, 1995, 51, 10389-10402). Mesylation of compound 128 with mehtanesulfonyl chloride and subsequent treatment with NaHCO₃ in absolute ethanol as described in Example 1 yields compound 129.

### Example 131

Compound 130 (Scheme 21): Benzoylation of compound 129 with benzoyl chloride in pyridine as reported in the literature yields compound 130 (Thrane et. al., Tetrahedron, 1995, 51, 10389-10402).

### Example 132

Compound 134 (Scheme 21): Compound 134 is prepared from compound 130 as described in Example 2, 3 and 8 for the synthesis of compound 6 from compound 3 (Scheme 1).

### Example 133

Scheme 22 is the synthetic scheme for monomers and intermediates described in Examples 133-136.

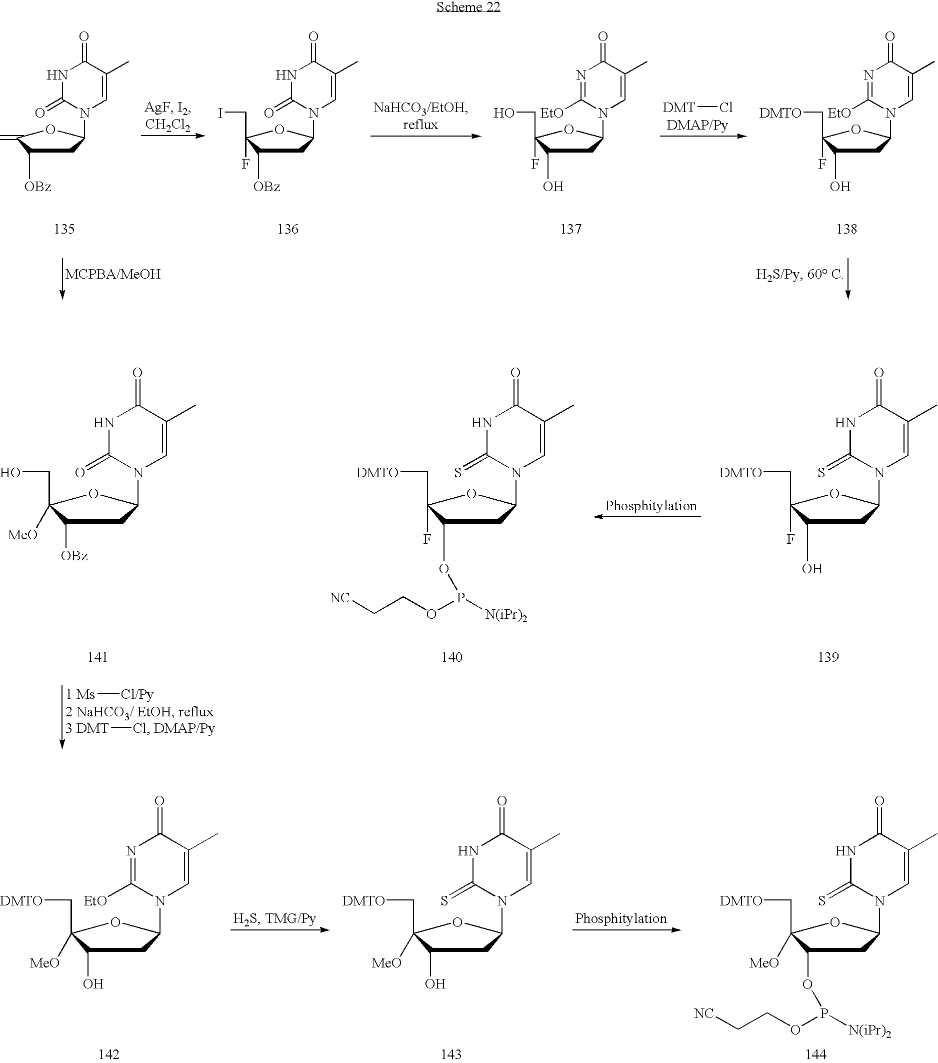

Compound 136 (Scheme 22): Compounds 135 is prepared according to the literature reports (Han et. al., Bull. Korean Chem. Soc., 2000, 21, 321-327). Compound 136 is obtained from compound 135 according to literature procedure (Guillerm et. al., Bioorg. Med. Chem. Lett., 1995, 5, 1455-1460).

### Example 134

Compound 140 (Scheme 22): Compound 140 is prepared from compound 136 as described in Examples 10, 11 and 12 for the synthesis of 5′-O-DMT-2′-deoxy-2′-fluoro-2-thio-5-methyluridine 3′-phosphoramidite (6, Example 1).

### Example 135

Compound 141 (Scheme 22): Compounds 135 is prepared according to the literature reports (Han et. al., Bull. Korean Chem. Soc., 2000, 21, 321-327). Compound 141 is obtained from compound 135 according to the procedure reported in the literature (Maag et. al., J. Med. Chem., 1992, 35, 1440-1451).

### Example 136

Compound 144 (Scheme 22): The desired phosphoramidate 144 is prepared from compound 140 as described in Examples 1, 2, 3 and 8 for the synthesis of compound 6 from compound 1 (Scheme 1)

### Example 137

Scheme 23 is the synthetic scheme for monomers and intermediates described in Examples 137-139.

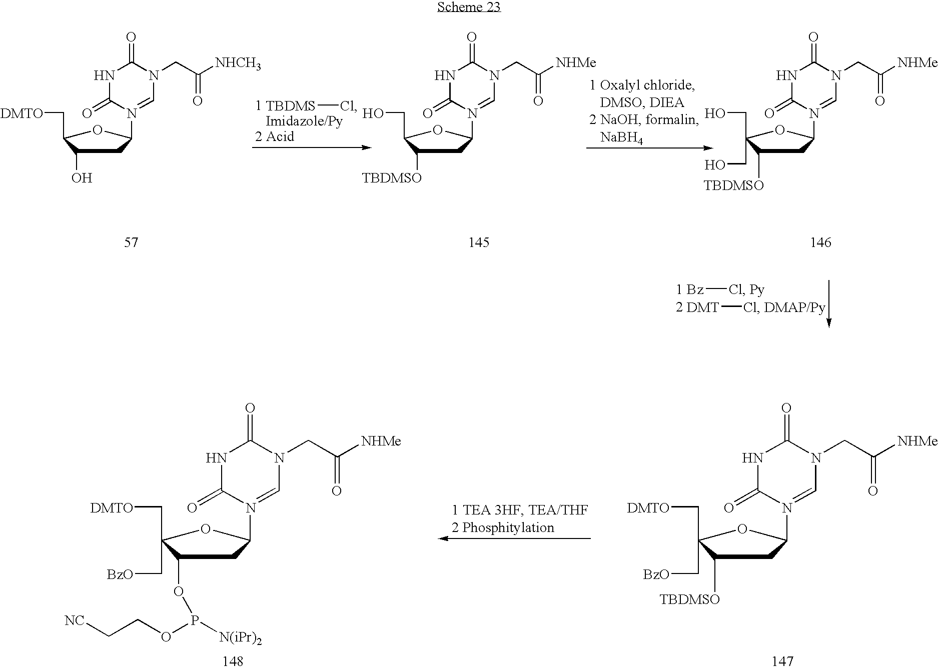

Compound 145 (Scheme 23): Compound 57 is stirred with 1.2 equivalent of TBDMS-Cl and 4 equivalent of imidazole in anhydrous pyridine for 6 h. The compound thus obtained is treated with acetic acid to obtain compound 145.

### Example 138

Compound 147 (Scheme 23): Compound 146 is obtained from compound 145 by following a literature procedure (Thrane et. al., Tetrahedron, 1995, 51, 10389-10402).

### Example 139

Compound 148 (Scheme 23): Treatment of compound 147 with triethylamine trihydrofluoride in the presence of triethylamine in THF and subsequent phosphitylation as described in Example 8 yields compound 148.

### Example 140

Scheme 24 is the synthetic scheme for monomers and intermediates described in Examples 140-144.

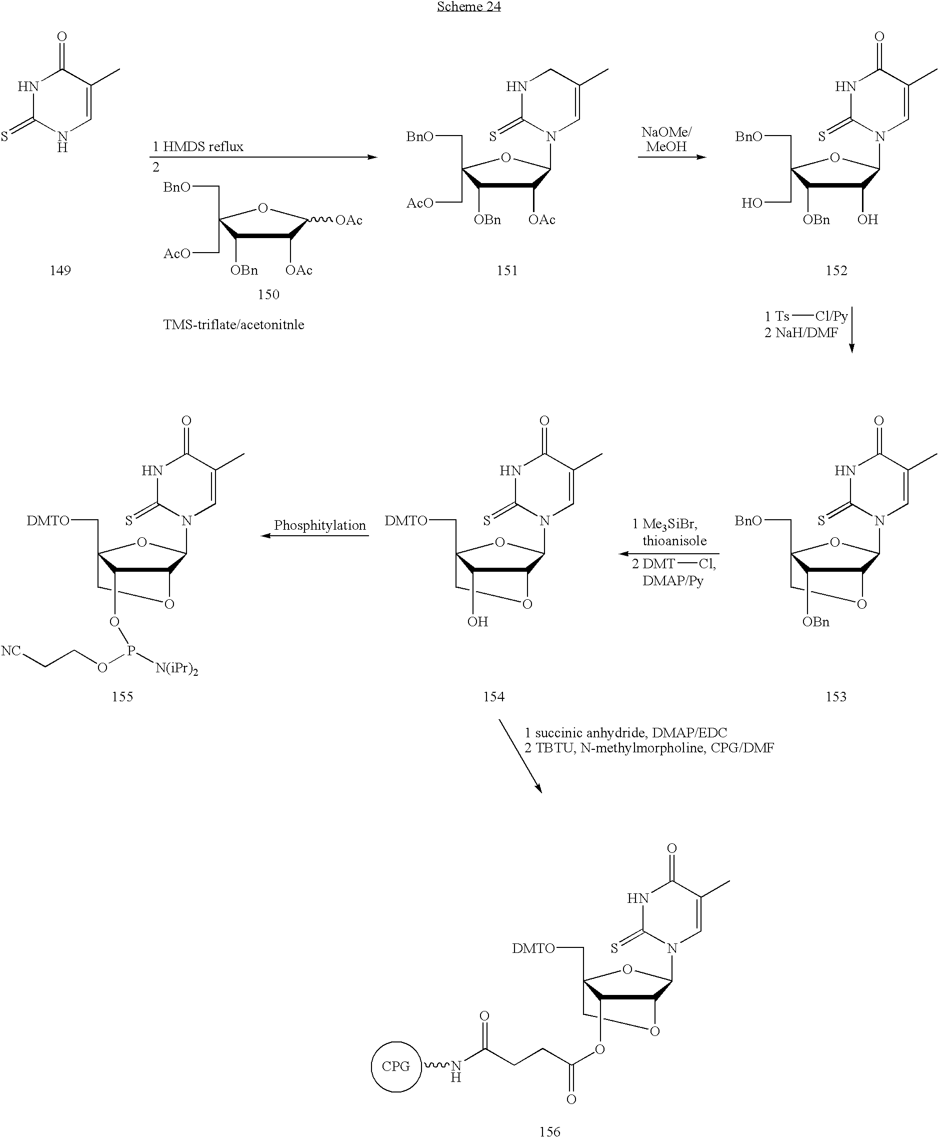

Compound 151 (Scheme 24): Compound 150 is prepared as reported in the literature (Koshkin et. al., Tetrahedron, 1998, 54, 3607-3630). 2-Thio-5-methyluracil (149) is refluxed in HMDS to obtain its corresponding dimethylsilylated derivative. The silylated derivative thus obtained is reacted with compound 150 according to a literature procedure (Koshkin et. al., Tetrahedron, 1998, 54, 3607-3630) to obtain compound 151.

### EXAMPLE 141

Compound 153 (Scheme 24): The desired compound 153 is prepared from compound 151 as reported by Koshikin et. al. (Tetrahedron, 1998, 54, 3607-3630).

### Example 142

Compound 154 (Scheme 24): Treatment of compound 153 with trimethylsilylbromide in the presence of thioanisole (Fujii et. al., Chem. Pharm. Bull., 1987, 35, 3880) removes the benzyl protection from the sugar moiety. The unprotected nucleoside thus obtained is reacted with DMT-Cl in the presence of DMAP as described in Example 2 yields compound 154.

### Example 143

Compound 155 (Scheme 24): Phsophitylation of compound 154 as described in Example 8 yields compound 155.

### Example 144

Compound 156 (Scheme 24): Controlled pore glass support is conjugated to 3′-hydroxyl function of compound 154 as described in Examples 91 and 92 gives the desired solid support 156.

### Example 145

Scheme 25 is the synthetic scheme for monomers and intermediates described in Examples 145-147, 165, and 166.

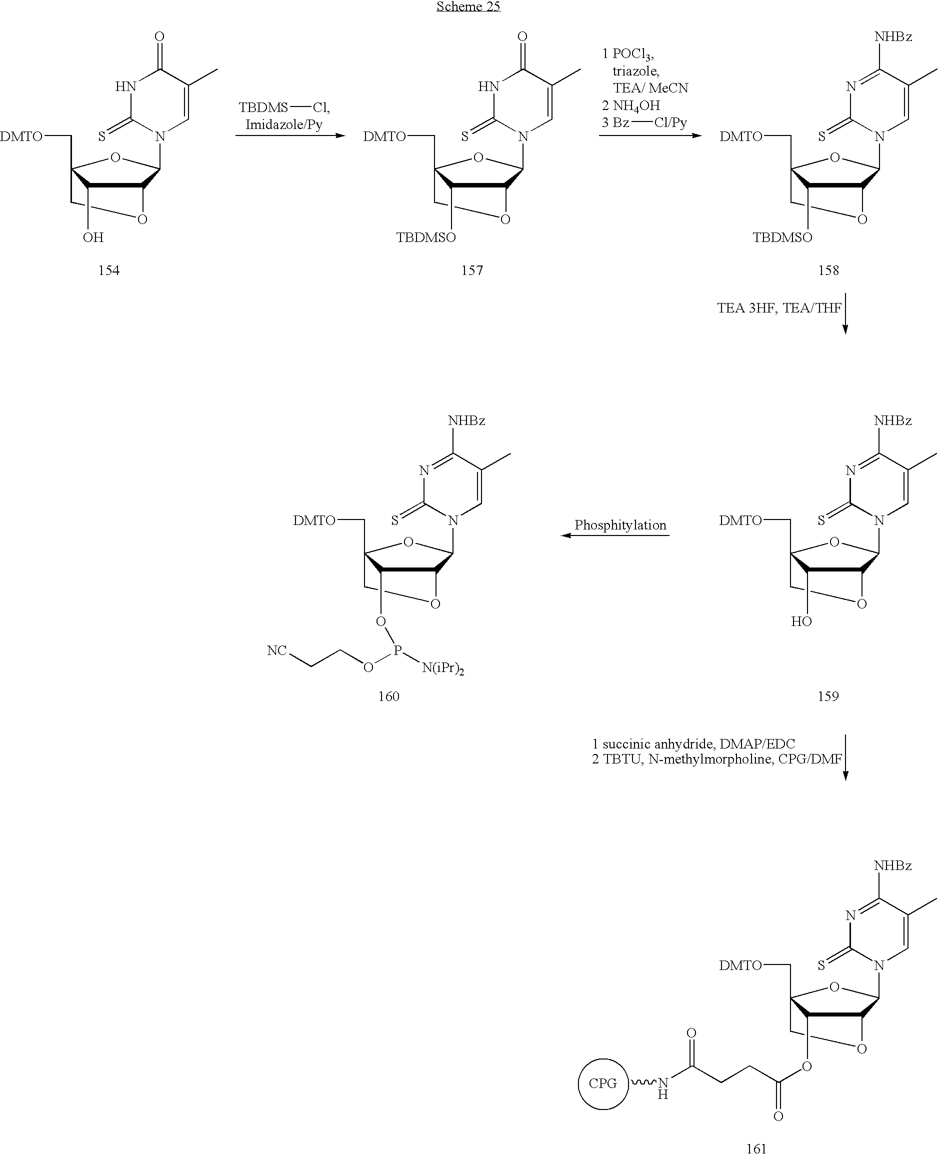

Compound 157 (Scheme 25): Treatment of compound 154 with TBDMS-Cl in the presence of imidazole in anhydrous pyridine as described in Example 15 gives compound 157.

### Example 146

Compound 160 (Scheme 25): Compound 160 is prepared from compound 157 as described in Examples 79 (appropriate parts of the experimental procedure), 80 and 81.

### Example 147

Compound 161 (Scheme 25): The desired solid support is obtained from compound 159 as described in Examples 91 and 92. Compound 159 is prepared from compound 157 as described in Examples 79 (appropriate parts of the experimental procedure) and 80.

### Example 148

Scheme 26 is the synthetic scheme for monomers and intermediates described in Examples 148-152.

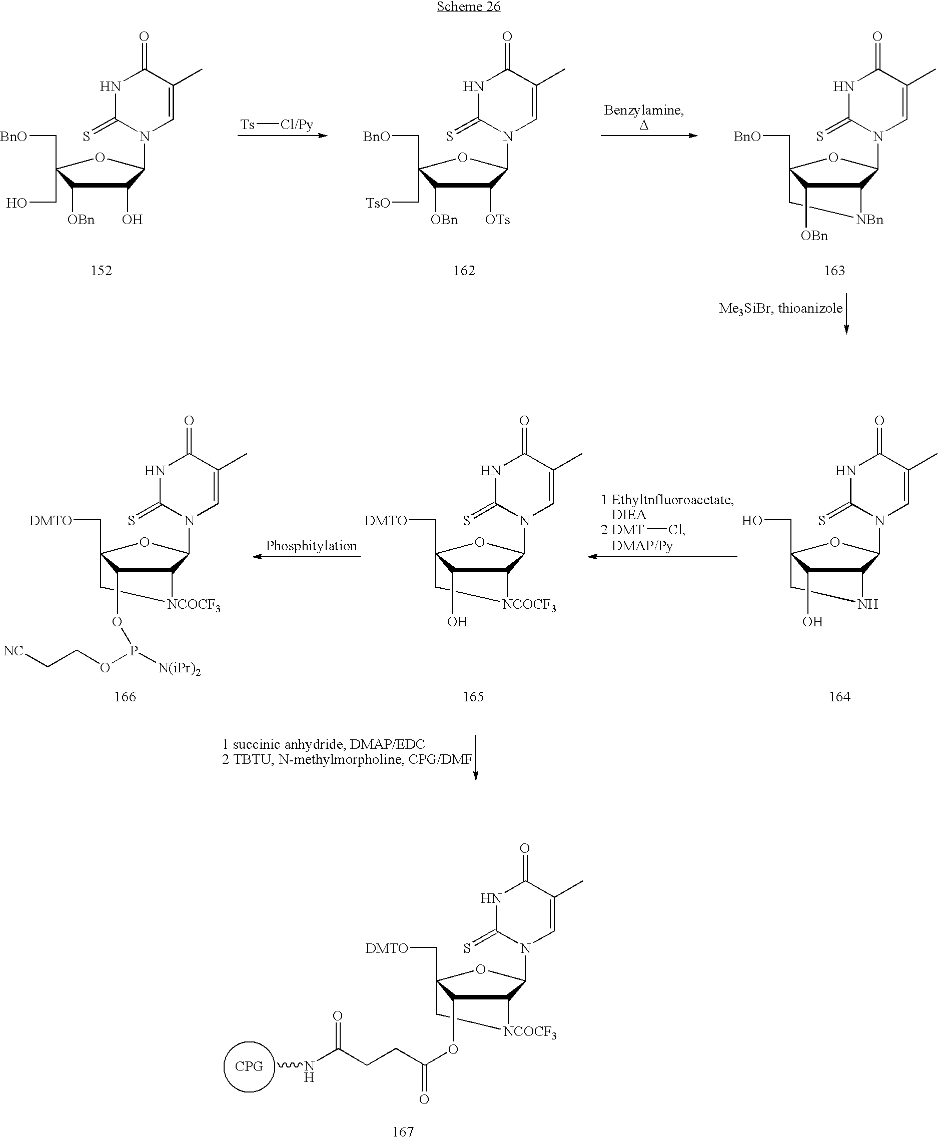

Compound 163 (Scheme 26): Compound 152 is prepared from compound 151 (Scheme 24) as reported in the literature (Koshkin et. al., Tetrahedron, 1998, 54, 3607-3630). The desired nucleoside 163 is prepared from compound 152 as reported in the literature (Singh et. al., J. Org. Chem., 1998, 63, 10035-10039).

### Example 149

Compound 164 (Scheme 26): Treatment of compound 163 with trimethylsilylbromide in the presence of thioanisole (Fujii et. al., Chem. Pharm. Bull., 1987, 35, 3880) yields compound 164.

### Example 150

Compound 165 (Scheme 26): Compound 165 is prepared from compound 164 as reported in the literature (Singh et. al., J. Org. Chem., 1998, 63, 10035-10039).

### Example 151

Compound 166 (Scheme 26): The phosphoramidite 166 is obtained from compound 165 according to the literature procedure (Singh et. al., J. Org. Chem., 1998, 63, 10035-10039).

### Example 152

Compound 167 (Scheme 26): Compound 167 is prepared from compound 165 as described in Examples 91 and 92.

### Example 153

Scheme 27 is the synthetic scheme for monomers and intermediates described in Examples 153-155.

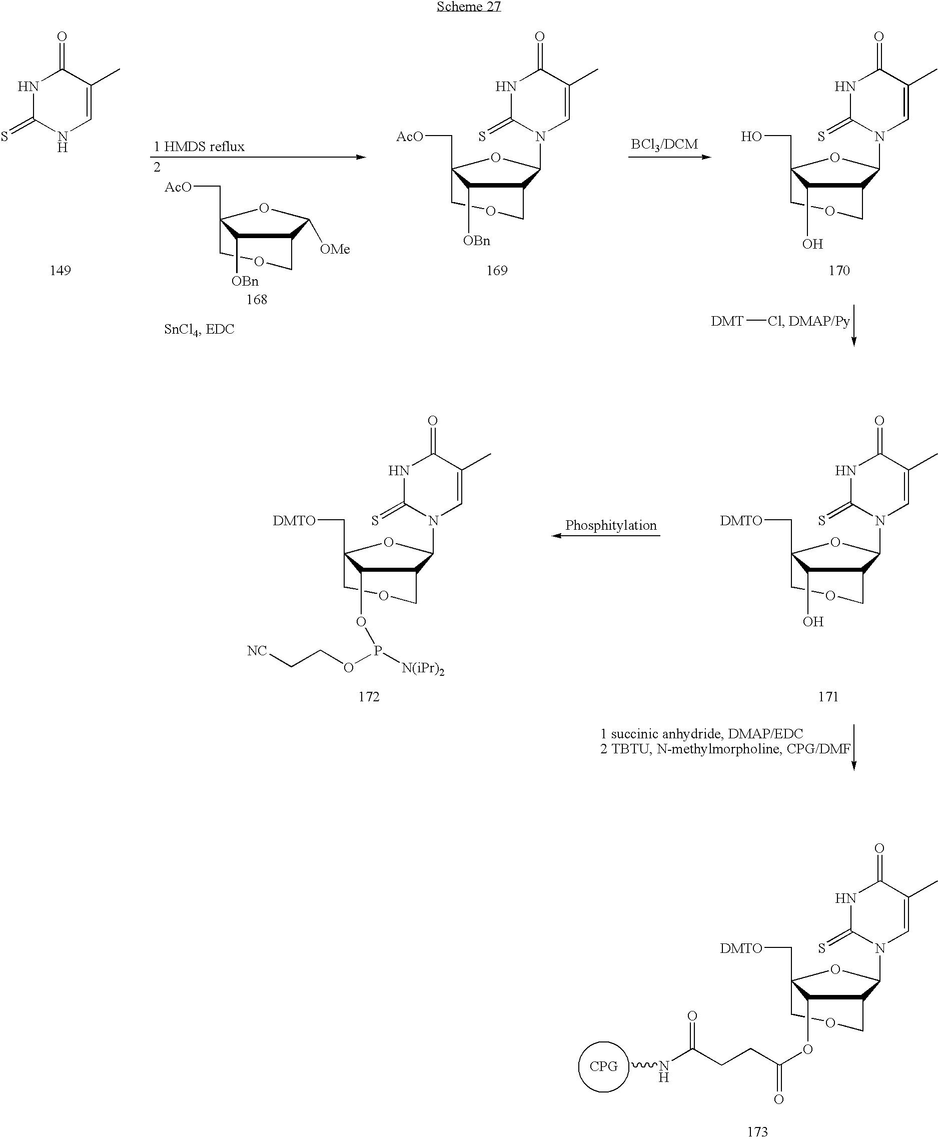

Compound 171 (Scheme 27): Compound 168 is prepared as reported in the literature (Wang et. al., Tetrahedron, 1999, 55, 7707-7724). The desired compound 171 is prepared from compound 168 and compound 149 according to the procedures reported by Wang et. al., (Tetrahedron, 1999, 55, 7707-7724).

### Example 154

Compound 172 (Scheme 27): Phosphitylation of compound 171 as described in Example 8 yields compound 172.

### Example 155

Compound 173 (Scheme 27): Controlled pore glass support is conjugated to 3′-hydroxyl function of compound 171 as described in Examples 91 and 92 gives the desired solid support 173.

### Example 156

Scheme 28 is the synthetic scheme for monomers and intermediates described in Examples 156-158.

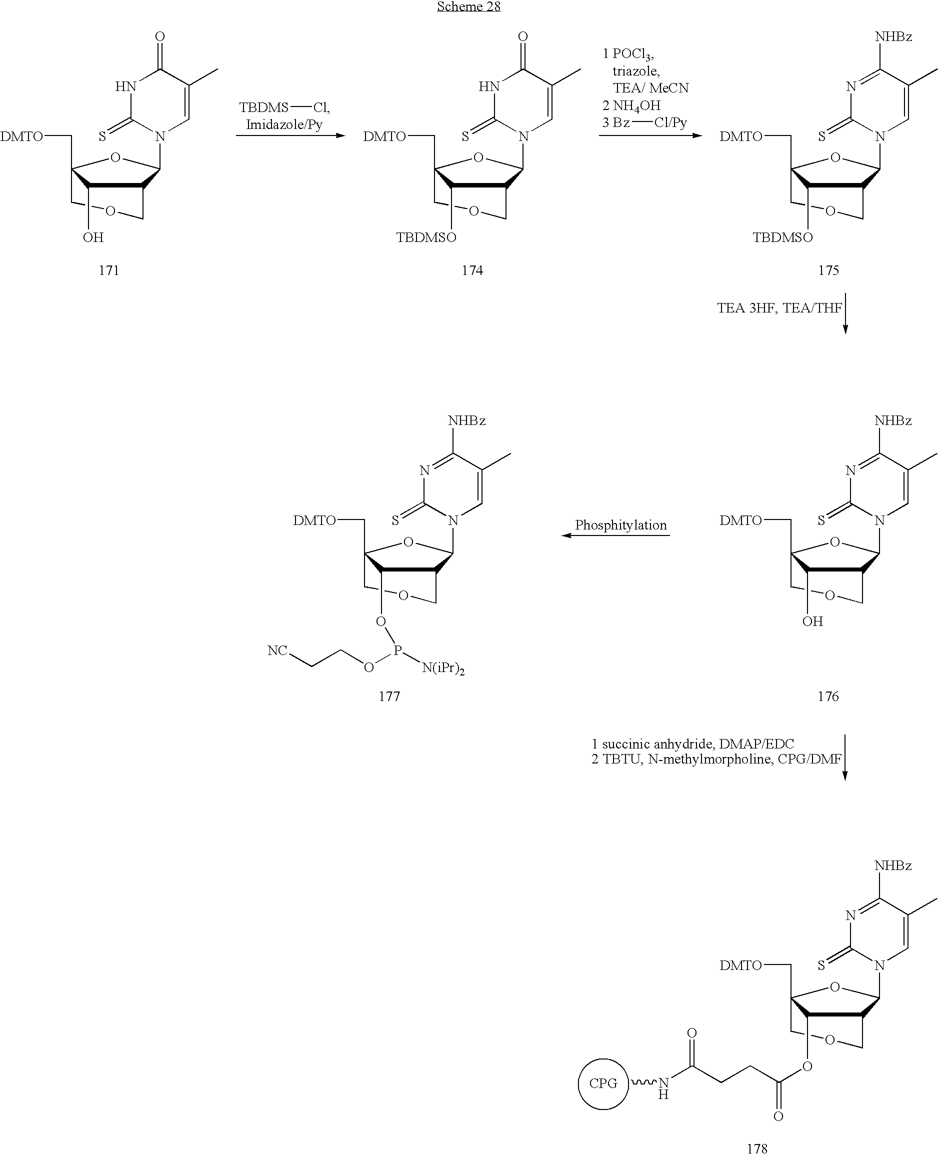

Compound 176 (Scheme 28): The desired compound 176 is prepared from compound 171 (obtained from Example 152) as described in Examples 79 (appropriate parts of the experimental procedure) and 80.

### Example 157

Compound 177 (Scheme 28): Phosphitylation of compound 176 as described in Example 8 yields compound 177.

### Example 158

Compound 178 (Scheme 28): Controlled pore glass support is conjugated to 3′-hydroxyl function of compound 176 as described in Examples 91 and 92 gives the desired solid support 178.

### Example 159

Scheme 29 is the synthetic scheme for monomers and intermediates described in Examples 159-163 and 186.

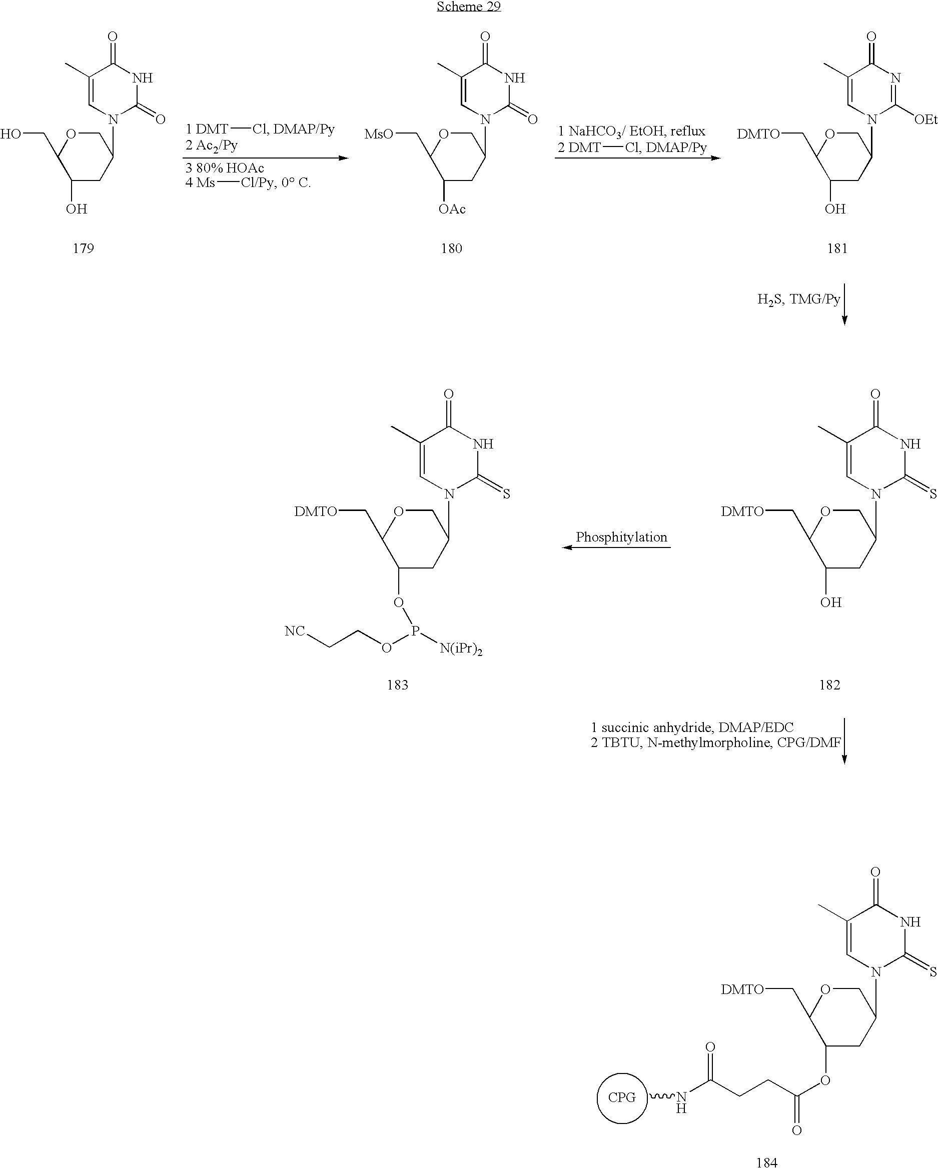

Compound 180 (Scheme 29): Compound 179 is prepared as reported in the literature (Wouters and Herdewijn, Bioorg. Med. Chem. Lett., 1999, 9, 1563-1566). Compound 179 is reacted with DMT-Cl in the presence of DMAP as described in Example 2 to obtain DMT derivative. Treatment of the DMT derivative compound 179 with acetic anhydride in anhydrous pyridine in the presence of DAMP gives acetylation at the secondary hydroxyl function. After acetylation, the DMT group is removed from the primary hydroxyl group by stirring in 80% aqueous acetic acid. Treatment of the product obtained with methanesulfonyl chloride in anhydrous pyridine at 0° C. yields the desired compound 180.

### Example 160

Compound 181 (Scheme 29): Compound 180 is refluxed in absolute ethanol in the presence of anhydrous NaHCO₃ as described in Example 1 (appropriate parts of the experimental procedure). The 2-ethoxy derivative thus forms is reacted with DMT-Cl in the presence of DMAP as described in Example 2 to yield compound 181.

### Example 161

Compound 182 (Scheme 29): Compound 181 is treated with H₂S in the presence of TMG in pyridine as described in Example 3 yields compound 182.

### Example 162

Compound 183 (Scheme 29): Phosphitylation of compound 182 as described in Example 8 yields the desired phosphoramidite 183.

### Example 163

Compound 184 (Scheme 29): Controlled pore glass (CPG) support is conjugated to 3′-hydroxyl function of compound 182 as described in Examples 91 and 92 gives the desired solid support 184.

### Example 164

Scheme 30 is the synthetic scheme for monomers and intermediates described in Example 164.

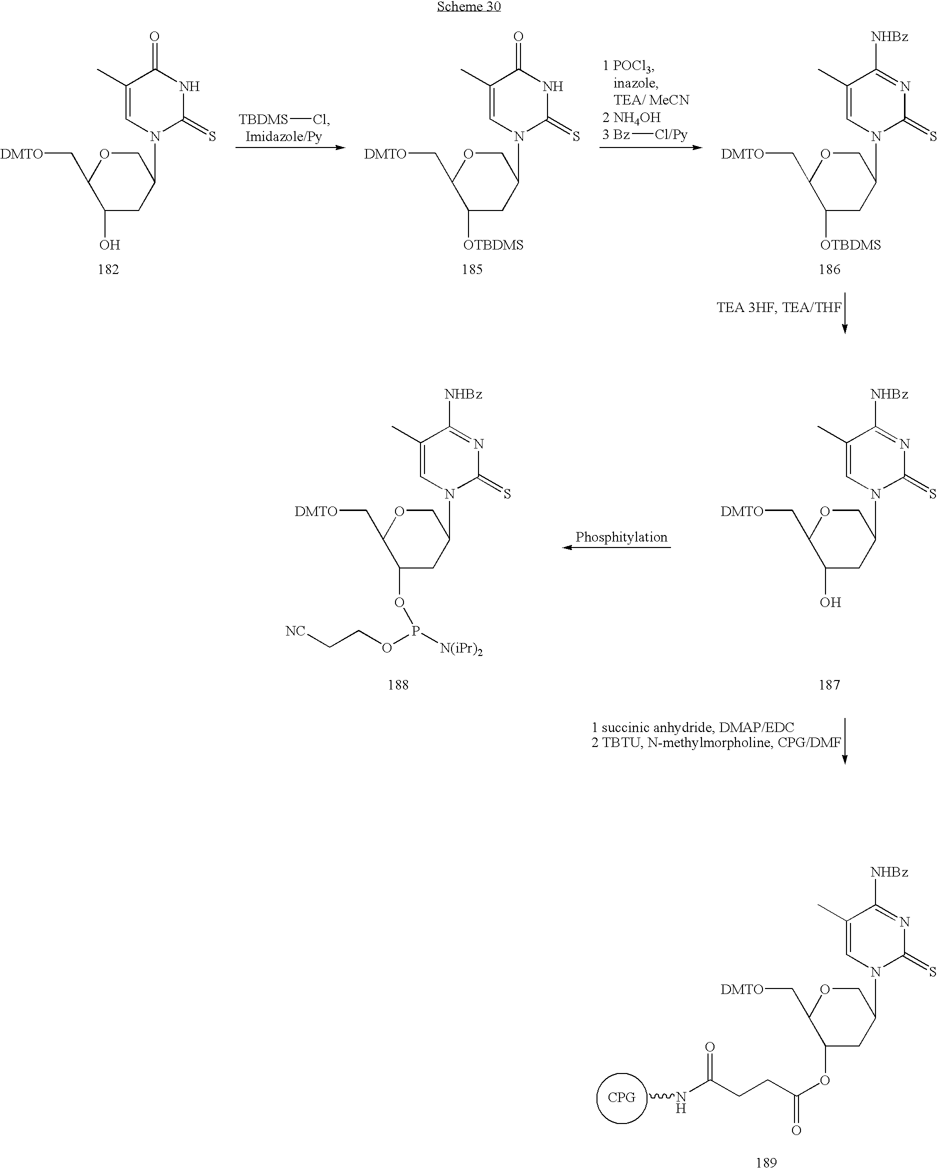

Compound 185 (Scheme 30): Treatment of compound 182 with TBDMS-CL in the presence of imidazole in anhydrous pyridine as described in Example 15 gives compound 185.

### Example 165

Compound 188 (Scheme 25): Compound 188 is prepared from compound 185 as described in Examples 79 (appropriate parts of the experimental procedure), 80 and 81.

### Example 166

Compound 189 (Scheme 25): The desired solid support 189 is obtained from compound 187 as described in Examples 91 and 92. Compound 187 is prepared from compound 185 as described in Examples 79 (appropriate parts of the experimental procedure) and 80.

### Example 167

Schemes 31a and 31b are the synthetic scheme for monomers and intermediates described in Examples 167-169.

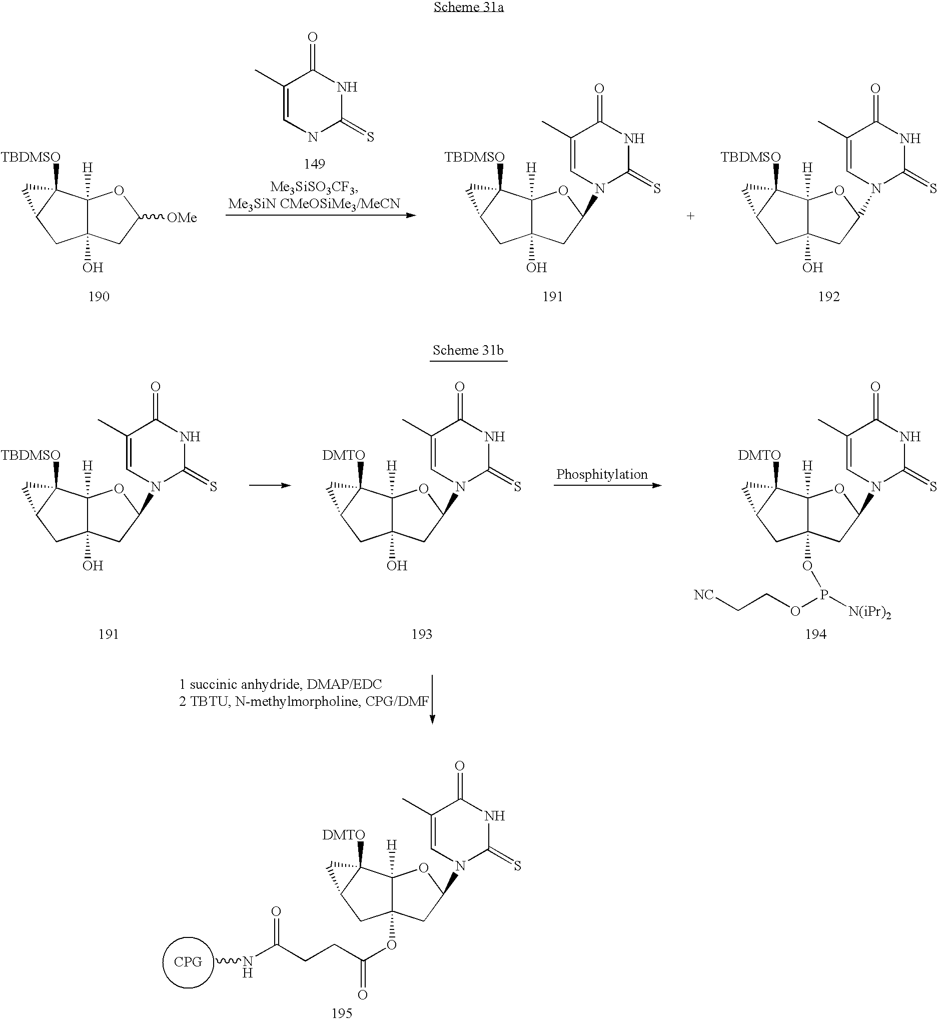

Compound 191 (Scheme 31A): Compound 190 is prepared as reported in the literature (Steffens and Leumann, Helv. Chim. Acta, 1997, 80, 2426-2439). Compound 191 is prepared from compounds 190 and 149 according to the reported procedure by Steffens and Leumann (Helv. Chim. Acta, 1997, 80, 2426-2439). The two stereo isomers formed are separated by flash column chramotography.

### Example 168

Compound 194 (Scheme 31b): Compound 194 is prepared from compound 191 as reported by by Steffens and Leumann (Helv. Chim. Acta, 1997, 80, 2426-2439).

### Example 169

Compound 195 (Scheme 31b): The desired solid support 195 is obtained from compound 193 as described in Examples 91 and 92. Compound 193 is prepared from compound 191 according to the literature procedure (Steffens and Leumann, Helv. Chim. Acta, 1997, 80, 2426-2439).

### Example 170

Scheme 32 is the synthetic scheme for monomers and intermediates described in Examples 170-173.

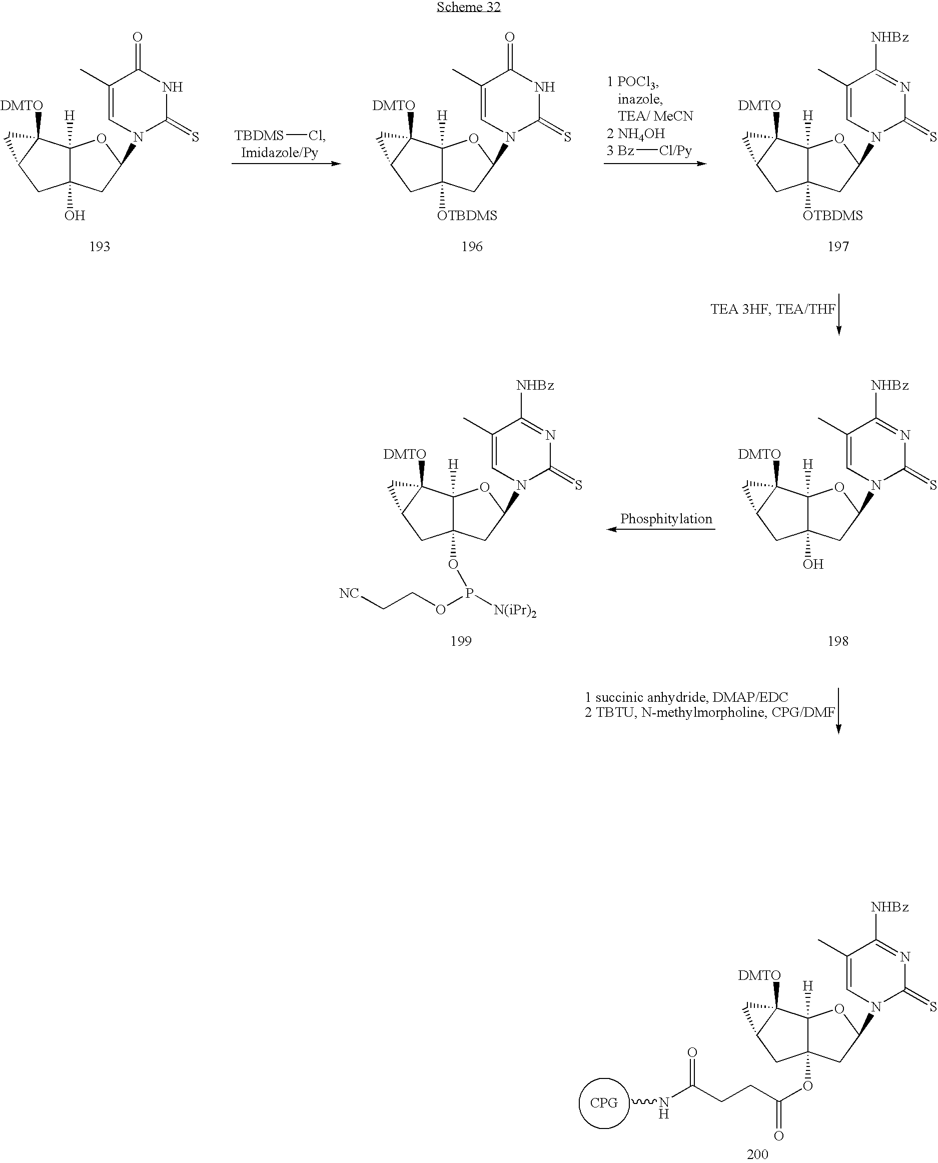

Compound 196 (Scheme 32): Treatment of compound 193 with TBDMS-Cl in the presence of imidazole in anhydrous pyridine as described in Example 15 gives compound 196.

### Example 171

Compound 198 (Scheme 32): Compound 198 is prepared from compound 196 as described in Examples 79 (appropriate parts of the experimental procedure) and 80.

### Example 172

Compound 199 (Scheme 32): Phosphitylation of compound 198 yields the desired phosphoramidite 199.

### Example 173

Compound 200 (Scheme 32): The desired solid support 200 is prepared from compound 198 in two steps as described in Examples 91 and 92.

### Example 174

Schemes 33a and 33b is the synthetic scheme for monomers and intermediates described in Examples 174-176.

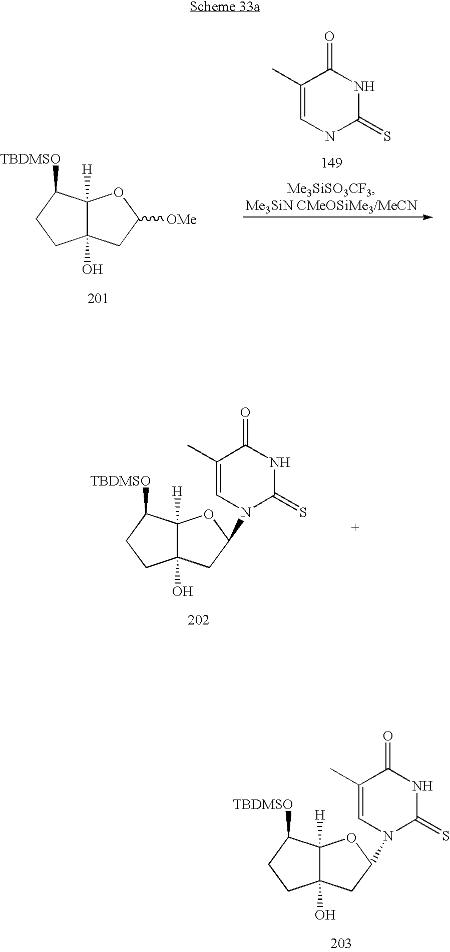

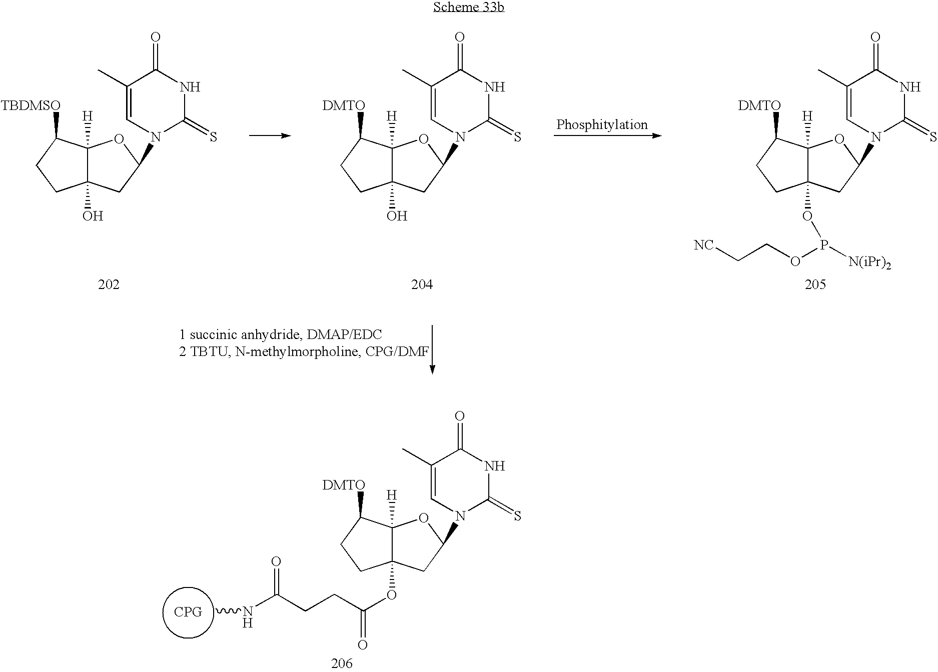

Compound 202 (Scheme 33A): Compound 201 is prepared as reported in the literature (Steffens and Leumann, Helv. Chim. Acta, 1997, 80, 2426-2439). Compound 202 is prepared from compounds 201 and 149 according to the reported procedure by Steffens and Leumann (Helv. Chim. Acta, 1997, 80, 2426-2439). The two stereo isomers formed are separated by flash column chramotography.

### Example 175

Compound 205 (Scheme 33b): Compound 205 is prepared from compound 202 as reported by by Steffens and Leumann (Helv. Chim. Acta, 1997, 80, 2426-2439).

### Example 176

Compound 206 (Scheme 33b): The desired solid support 206 is obtained from compound 204 as described in Examples 91 and 92. Compound 204 is prepared from compound 202 according to the literature procedure (Steffens and Leumann, Helv. Chim. Acta, 1997, 80, 2426-2439).

### Example 177

Scheme 34 is the synthetic scheme for monomers and intermediates described in Examples 177-180.

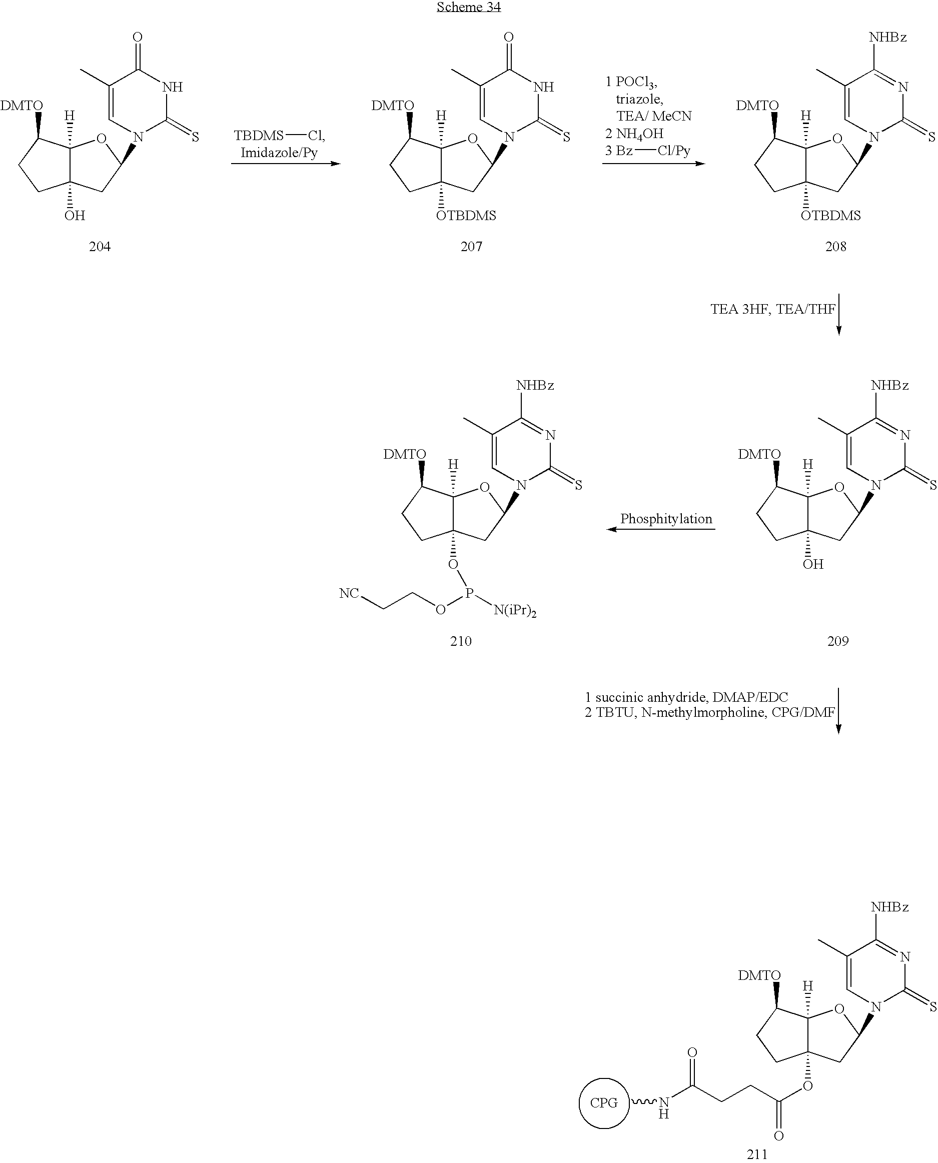

Compound 207 (Scheme 34): Treatment of compound 204 with TBDMS-Cl in the presence of imidazole in anhydrous pyridine as described in Example 15 gives compound 207.

### EXAMPLE 178

Compound 209 (Scheme 34): Compound 209 is prepared from compound 207 as described in Examples 79 (appropriate parts of the experimental procedure) and 80.

### Example 179

Compound 210 (Scheme 34): Phosphitylation of compound 209 yields the desired phosphoramidite 210.

### Example 180

Compound 211 (Scheme 34): The desired solid support 211 is prepared from compound 209 in two steps as described in Examples 91 and 92.

### EXAMPLE 181

Scheme 35 is the synthetic scheme for monomers and intermediates described in Examples 181-185 and 187.

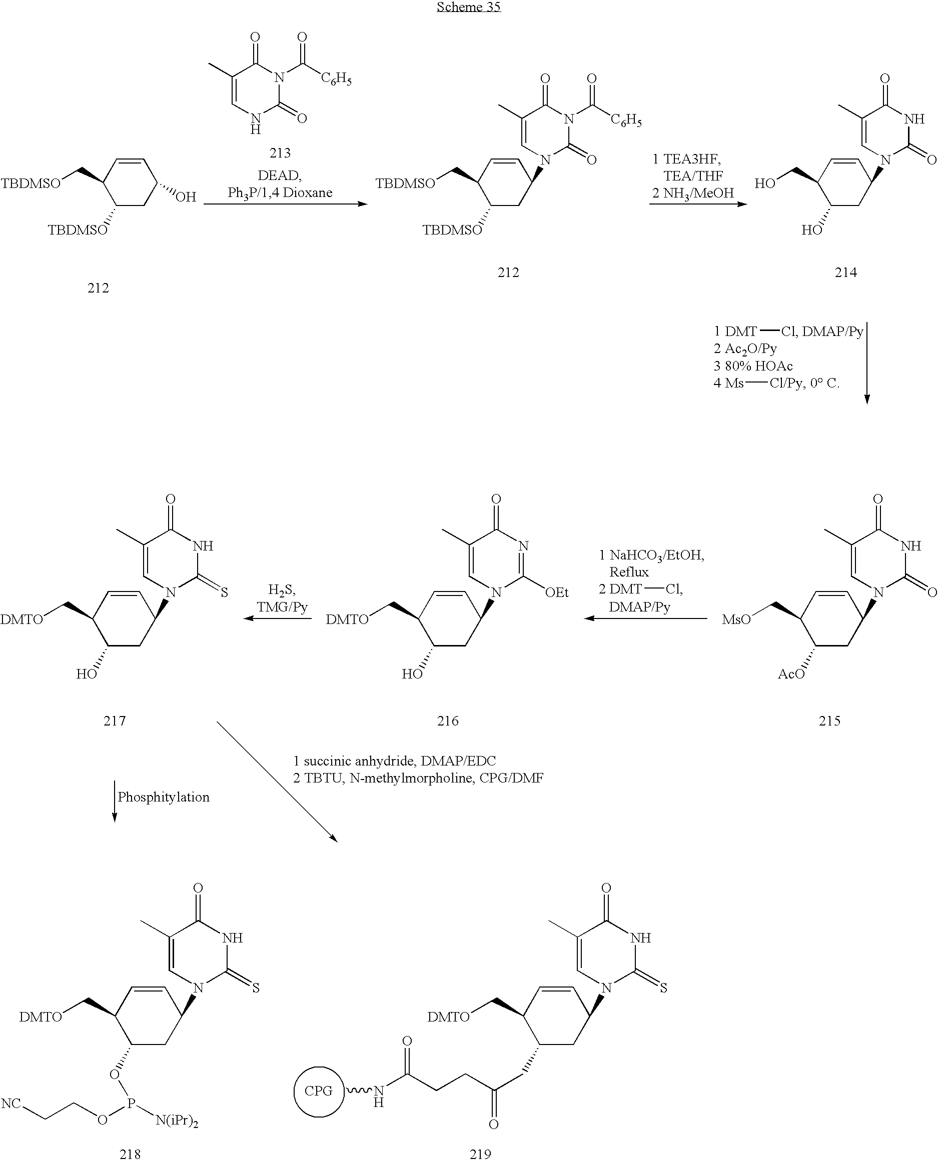

Compound 213 (Scheme 35): Compound 212 is prepared as reported in the literature (Wang and Herdewijn, J. Org. Chem., 1999, 64, 7820-7827). N3-Benzoylthymine is prepared as reported in the literature (Song, et. al., J. Med. Chem., 2001, 44, 3985-3993). Reaction of compound 212 with compound 213 in the presence of DEAD and Ph₃P as reported in the literature (Song, et. al., J. Med. Chem., 2001, 44, 3985-3993) yields compound 213.

### Example 182

Compound 214 (Scheme 35): Desilylation of compound 213 as described in Example 80 (appropriate parts of the experimental procedure). The desilylated product thus obtained is treated with methanolic ammonia to obtain the desired compound 214.

### Example 183

Compound 215 (Scheme 35): The desired compound 215 is prepared from compound 214 in 4 steps as described in Example 155 for the synthesis of compound 180.

### Example 184

Compound 216 (Scheme 35): Compound 215 is refluxed in absolute ethanol in the presence of anhydrous NaHCO₃ as described in Example 1 (appropriate parts of the experimental procedure). The 2-ethoxy derivative thus forms is reacted with DMT-Cl in the presence of DMAP as described in Example 2 to yield compound 216.

### Example 185

Compound 217 (Scheme 35): Compound 216 is treated with H₂S in the presence of TMG in pyridine as described in Example 3 yields compound 217.

### Example 186

Compound 218 (Scheme 29): Phosphitylation of compound 217 as described in Example 8 yields the desired phosphoramidite 218.

### Example 187

Compound 219 (Scheme 35): Controlled pore glass (CPG) support is conjugated to 3′-hydroxyl function of compound 217 as described in Examples 91 and 92 gives the desired solid support 219.

### Example 188

Scheme 36 is the synthetic scheme for monomers and intermediates described in Examples 188-190.

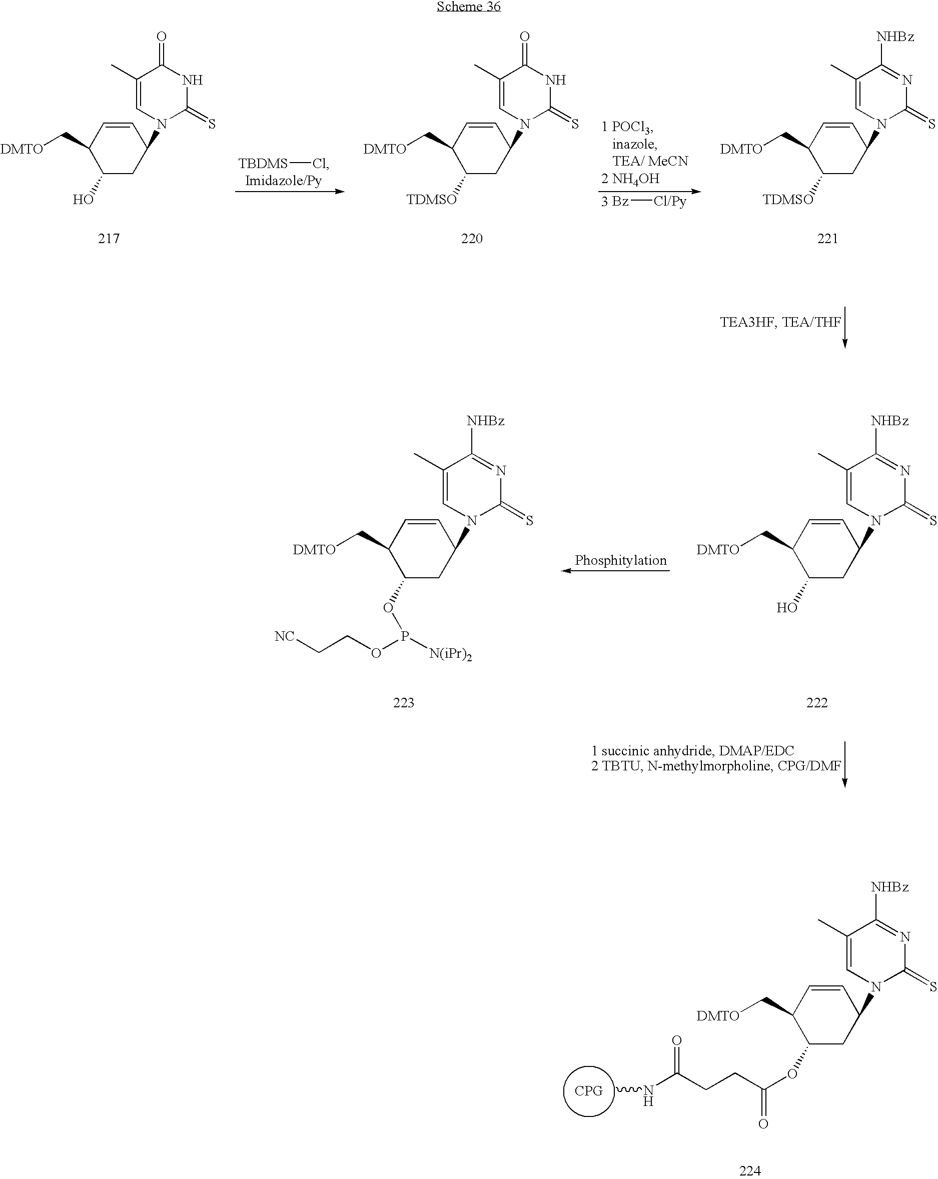

Compound 220 (Scheme 36): Treatment of compound 217 with TBDMS-Cl in the presence of imidazole in anhydrous pyridine as described in Example 15 gives compound 220.

### Example 189

Compound 223 (Scheme 36): Compound 223 is prepared from compound 220 as described in Examples 79 (appropriate parts of the experimental procedure), 80 and 81.

### Example 190

Compound 224 (Scheme 36): The desired solid support 224 is obtained from compound 222 as described in Examples 91 and 92. Compound 222 is prepared from compound 220 as described in Examples 79 (appropriate parts of the experimental procedure) and 80.

### Example 191

Scheme 37 is the synthetic scheme for monomers and intermediates described in Examples 191-195.

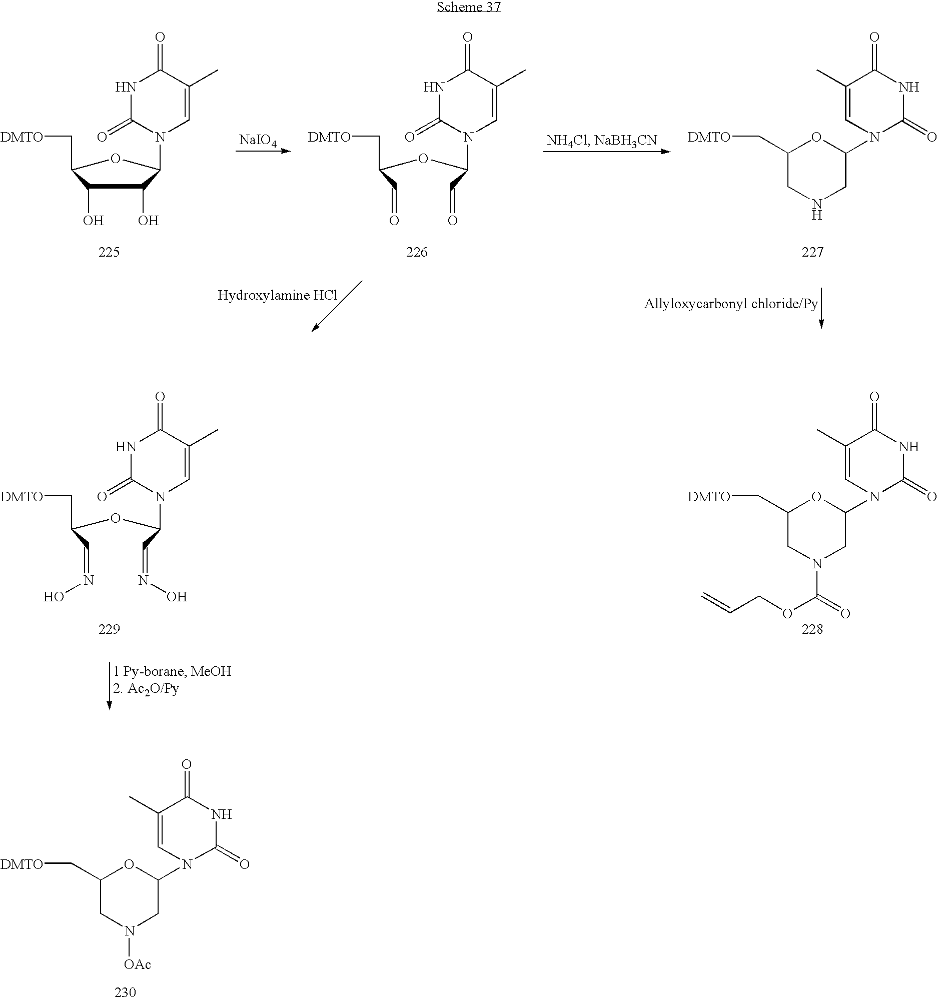

Compound 226 (Scheme 37): NaIO₄ oxidation of 5′-O-DMT-5-methylurdine yields the desired dialdehyde 226.

### Example 192

Compound 227 (Scheme 37): Compound 226 is treated with one molar equivalent of ammonium chloride in the presence of excess NaBH₃CN in methanol to obtain compound 226.

### Example 193

Compound 228 (Scheme 37): Compound 227 upon treatment with allylchloroformate in anhydrous pyridine at 0° C. (Corey and Suggs, J. Org. Chem., 1973, 38, 3223) yields the desired compound 228.

### Example 194

Compound 229 (Scheme 37): Compound 229 is obtained from compound 226 according to the reported procedure (Tronchet, et. al., Tetrahedron Lett., 1991, 32, 4129-32).

### Example 195

Compound 230 (Scheme 37): Reduction of compound 229 as reported in the literature (Tronchet, et. al., Nucleosides Nucleotides, 1993, 12, 615-629) and subsequent treatment with acetic anhydride in anhydrous pyridine yields the desired compound 230.

### Example 196

Scheme 38 is the synthetic scheme for monomers and intermediates described in Examples 196-201.

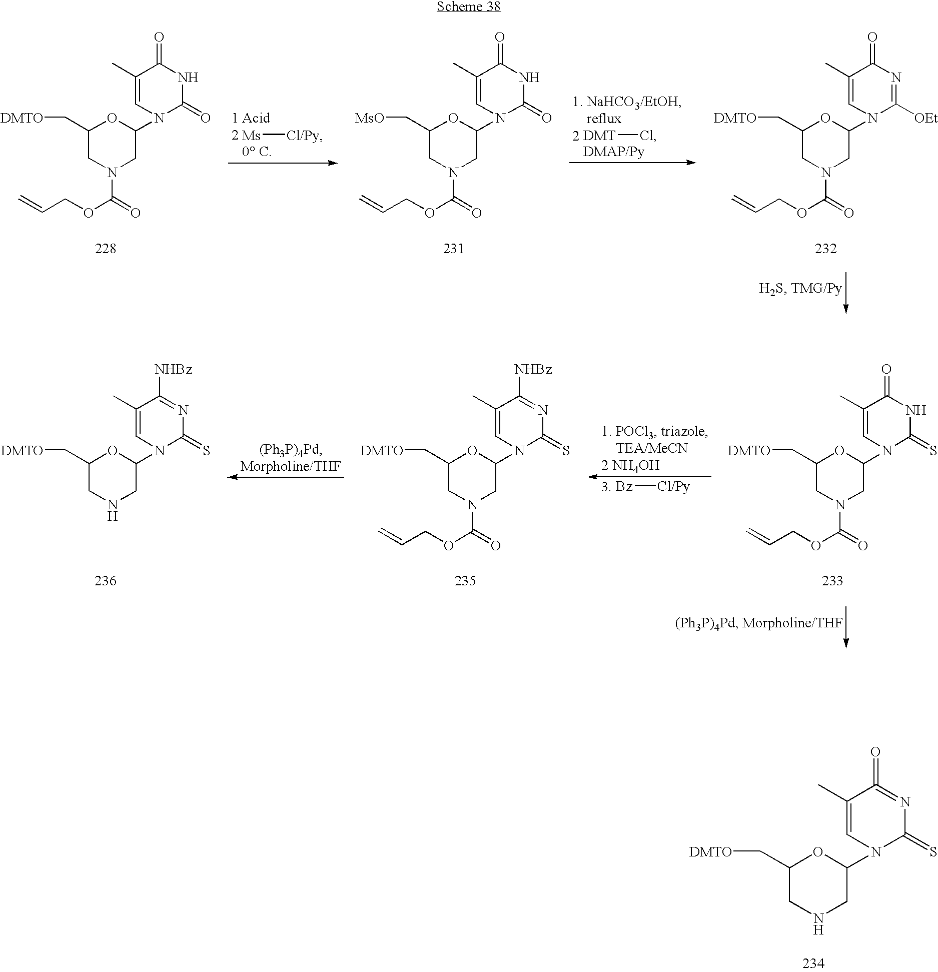

Compound 231 (Scheme 38): Acid treatment of compound. 228 gives the corresponding hydroxy compound. The free hydroxyl thus formed is converted into its methane sulfonate 231 by reacting with Ms-Cl in pyridine at 0° C.

### Example 197

Compound 232 (Scheme 38): Compound 231 is refluxed in absolute ethanol in the presence of anhydrous NAHCO₃ as described in Example 1 to obtain the corresponding 2-ethoxy derivative. The ethoxy derivative formed is treated with DMT-Cl in the presence of DMAP as described in Example 2 to obtain compound 232.

### Example 198

Compound 233 (Scheme 38): Compound 232 is converted to the desired 2-thio analogue 233 by reacting with H₂S in the presence of TMG in anhydrous pyridine as described in Example 3.

### Example 199

Compound 234 (Scheme 38): Compound 233 is treated with 10 molar excess of morpholine and catalytic amount of tetrakistriphenylphosphine palladium(0) in anhydrous THF (Kunz and Waldmann, Angew. Chem. Int. Ed. Engl., 1984, 23, 71-72) to obtain the desired compound 234.

### Example 200

Compound 235 (Scheme 38): Compound 235 is prepared from compound 233 as described in Example 79 (second part of the procedure) and Example 80 (first part of the procedure).

### Example 201

Compound 236 (Scheme 38): The allylcarbamate protection of compound 235 is removed as described in Example 193 to obtain the desired compound 236.

### Example 202

Scheme 39 is the synthetic scheme for monomers and intermediates described in Examples 202-205.

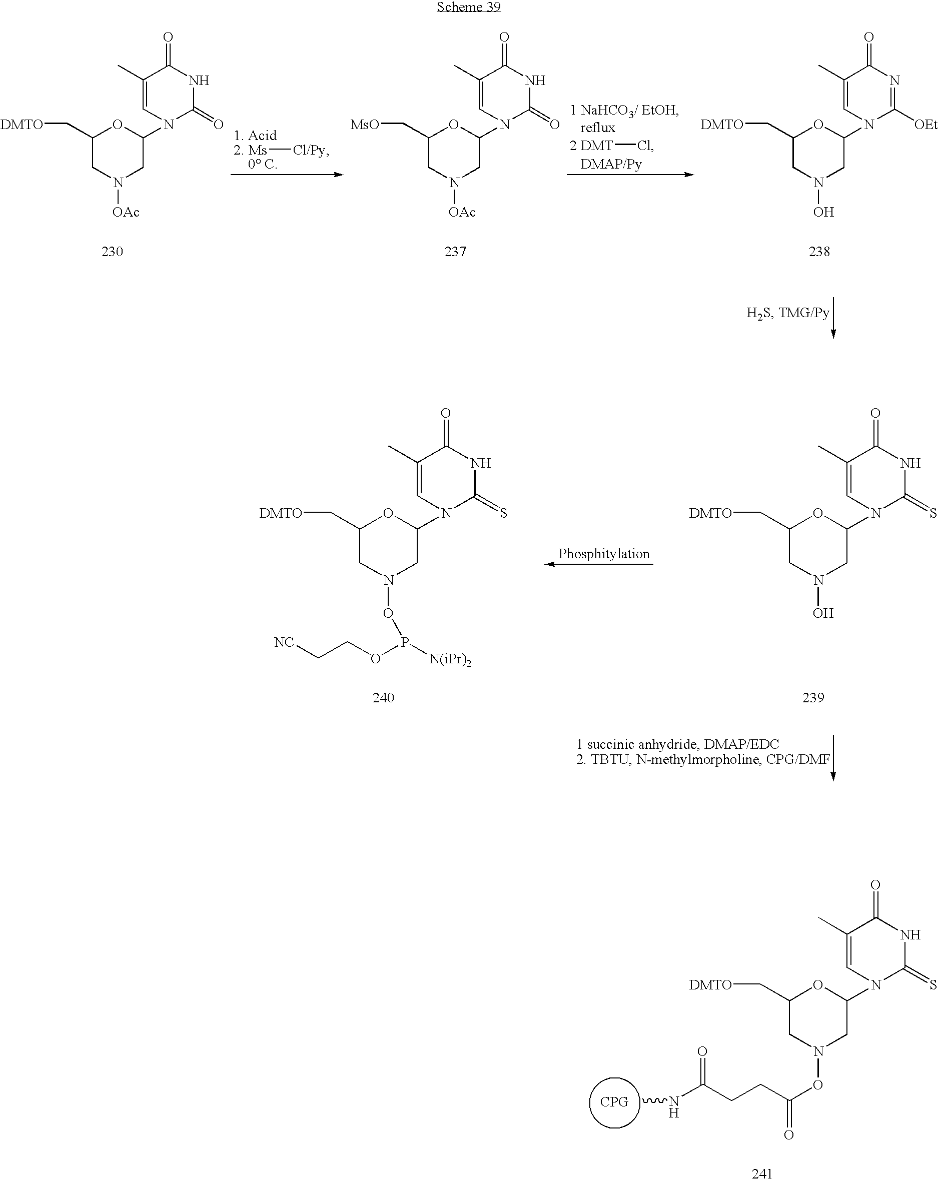

Compound 237 (Scheme 39): Compound 237 is prepared from compound 230 as described in Example 196.

### Example 203

Compound 239 (Scheme 39): Compound 239 is prepared from compound 237 according to the procedure described in Examples 197 and 198

### Example 204

Compound 240 (Scheme 39): Phosphitylation of compound 239 as described in Example 8 yields the desired phosphoramidite 240.

### Example 205

Compound 241 (Scheme 39): Conjugation of compound 139 to control pore glass (CPG) support as described in Examples 91 and 92 yields the desired solid support 241.

### Example 206

Scheme 40 is the synthetic scheme for monomers and intermediates described in Examples 206-208.

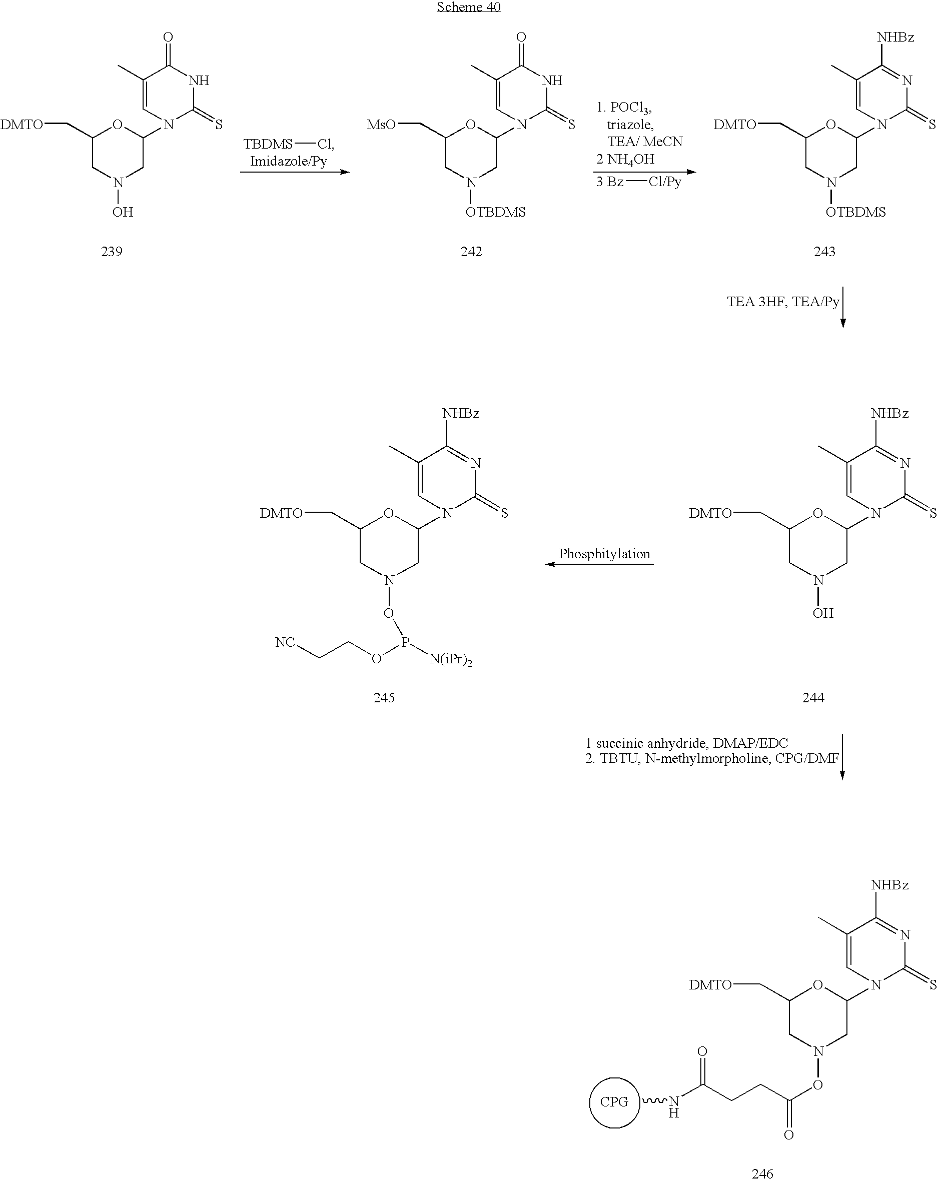

Compound 242 (Scheme 40): Treatment of compound 239 with TBDMS-Cl in the presence of imidazole in anhydrous pyridine as described in Example 15 gives compound 242.

### Example 207

Compound 245 (Scheme 40): Compound 245 is prepared from compound 241 as described in Examples 79 (appropriate parts of the experimental procedure), 80 and 81.

### Example 208

Compound 246 (Scheme 40): The desired solid support 246 is obtained from compound 244 as described in Examples 91 and 92. Compound 244 is prepared from compound 242 as described in Examples 79 (appropriate parts of the experimental procedure) and 80.

### Example 209

Synthesis of 2′-O-MOE-2-thio modified Oligonucleotides. A 0.1 M solution of the amidite 6 (R=OCH₂CH₂OCH₃, X=CH₃) in anhydrous acetonitrile was used for the synthesis of modified oligonucleotides. The oligonucleotides were synthesized on functionalized controlled pore glass (CPG) on an automated solid phase DNA synthesizer. CPG functionalized with 2′-O-MOE-2-thio modified nucleosides were used wherever necessary. For incorporation of 2′-O-MOE-2-thio phosphoramidite solutions were delivered in two portions, each followed by a 5 min coupling wait time. All other steps in the protocol supplied by the manufacturer were used without modification. Oxidation of the internucleotide phosphite to the phosphate was carried out using 10% tertbutylhydroperoxide in acetonitrile with 10 min waiting time. The Beaucage reagent (0.1 M in acetonitrile) was used as a sulfurizing agent. Oligonucleotides were synthesized DMT on mode. The coupling efficiencies were more than 97%. After completion of the synthesis, the solid support was suspended in aqueous ammonium hydroxide (30 wt %, 2 mL for 2 micromole synthesis) and kept at room temperature for 2 h. The supernatant was decanted, the CPG was washed with additional 1 mL of aqueous ammonia. Combined ammonia solution was heated at 55° C. for 6 h. Concentrated the solution to half of the volume. Adjusted the pH of the solution to 8 and the crude oligonucleotides were purified by high performance liquid chromatography (HPLC, C-4 column, Waters, 7.8×300 mm, A=100 mM ammonium acetate, B=acetonitrile, 5-60% of B in 55 min, flow 2.5 mL min-1, X 260 nm). Fractions containing the full length oligonucleotides were pooled together and pH of the solution was adjusted to 4.2 with acetic acid and kept at room temperature for 24 h. An aliquot was withdrawn and analyzed by HPLC on C-4 column (condition same as above) to asses the completion of the detritylation reaction. Neutralized the solution with ammonia and desalted by HPLC on a C-4 column to yield 2′-modified oligonucleotides in 30-40% isolated yield. The oligonucleotides were characterized by ESMS and HPLC and Capillary Gel Electrophoresis assessed their purity.

TABLE 1
HPLC and Mass Spectral Analysis of the 2′-O-MOE-2-thio oligonucleotides
used for Tm analysis

| | | | | HPLC |
|---|---|---|---|---|
| Seq. | | | | Retention |
| ID | | Mass | | Time, |
| No. | Sequences | Calcd | Found | min.^{a} |
| 1 | 5’ T*oCoCoAoGoGoT*oGoT*oCoCoGoCoAo | 5194.1 | 5193.2 | 24.30 |
| 2 | T*oC3’ | 5776.6 | 5775.98 | 32.04 |
| | 5’GoCoGoT*oT*oT*oT*oT*oT*oT*oT*o | | | |
| | T*oT*oGoCoG 3’ | | | |

^{a}Water C-4, 3.9 × 300 mm, A = 50 mM triethylammonium acetate, pH 7, B = acetonitrile, 5 to 60% B in 55 min, flow 1.5 mL min⁻¹, λ = 260 nm, T* = 2'-O-[2-(methoxy)ethyl}-2-thio-5-methyluridine

### Example 210

Evaluation of Hybridization of 2′-O-MOE-2-thiopyrimidine Modified Oligonucleotides to Complementary RNA and DNA by Thermal Denaturation Studies.

Thermal denaturation studies of duplex of oligonucleotides containing 2′-OMOE-2-thio moiety and complementary RNA have shown 3.2° C. per modification (Table 2) Tm enhancement compared to 2′-deoxy oligonucleotide phosphodiesters. This translates into 4° C. per modification increase in Tm per modification compared to 2′-deoxy oligonucleotide phosphorothioates. The 2′-O-MOE-2-thio modified oligonucleotides showed 2° C. per modification higher Tm compared to 2′-O-MOE modified oligonucleotides. Table 3 shows Tm value of modified olgonucleotide 2 against complementary DNA. This data suggest that 2′-O-MOE-2-thio modified oligonucleotide form less stable duplex with DNA than RNA.

TABLE 2
Effect of the 2’-O-MOE-2-thio and 2’-O-MOE modification on duplex stability
against complementary RNA targets

| Seq. | | | ΔTm/ |
|---|---|---|---|
| ID | | Tm | modification |
| No. | sequence | ° C. | ° C. |
| 1 | 5’ T*oCoCoAoGoGoT*oGoT*oCoCoGoCoAoT*oC 3’ | 74.1 | 2.92 |
| 2 | 5’ GoCoGoT*oT*oT*oT*oT*oT*oT*oT*oT*oT*oGoC | 82.90 | 3.43 |
| | oG 3’ | | |
| 3 | 5’ ToCoCoAoGoGoToGoToCoCoGoCoAoToC 3’ | 62.4 | |
| 4 | 5’ T^{&}oCoCoAoGoGoT^{&}oGoT^{&}oCoCoGoCoAoT^{&}oC 3’ | 65.9 | 0.88 |
| 5 | 5’ GoCoGoToToToToToToToToToToGoCoG 3’ | 48.50 | |
| 6 | 5’ GoCoGoT^{&}oT^{&}oT^{&}oT^{&}oT^{&}oT^{&}oT^{&}oT^{&}oT^{&}oT^{&}o | 60.0 | 1.15 |
| | G^{&}oCoG 3’ | | |

T* = 2’-O-[2-(methoxy)ethyl}-2-thio-5-methyluridine, T^{&} = 2’-O-[2(methoxy)ethyl}-5-methyluridine, o = P = O

TABLE 3
Effect of the 2’-O-MOE-2-thio and 2’-O-MOE modifications on duplex stability
against complementary DNA targets

| Seq. | | | |
|---|---|---|---|
| ID | | Tm | ΔTm/unit |
| No. | sequence | ° C. | ° C. |
| 2 | 5’ GoCoGoT*oT*oT*oT*oT*oT*oT*oT*oT*oT*oGoCo | 73.5 | 1.93 |
| | G3’ | | |
| 5 | 5’ GoCoGoToToToToToToToToToToGoCoG 3’ | 54.2 | |
| 6 | 5’ GoCoGoT^{&}oT^{&}oT^{&}oT^{&}oT^{&}oT^{&}oT^{&}oT^{&}oT^{&}oT^{&}o | 42.4 | −1.12 |
| | G^{&}oCoG 3’ | | |

T* = 2’-O-[2-(methoxy)ethyl]-2-thio-5-methyluridine, T^{&} = 2’-O-[2-(methoxy)ethyl]-5-methyluridine o = P = O

### Example 211

2′-O-MOE-2-thio modified antisense oligonucleotides for in vitro and in vivo evaluation: Oligonucleotide Gapmers targeted to Mouse p38 alpha, PTEN and Mouse TRADD and hemimer targeted to m-A-raf with 2′-O-MOE-2-thio modifications are synthesized (Table 4). Fully modified oligonucleotides with 2′-O-MOE-2-thio modifications (Table 4) are also synthesized for evaluating their efficacy in non RNase H mediated antisense applications. The efficacy of these antisense oligonucleotides to reduce the messages is evaluated in vitro and in vivo.

TABLE 4
Oligonucleotides with 2’-O-MOE-2-thio and 2’-O-MOE modifications for in vitro and
in vivo evaluation

| Seq. | | |
|---|---|---|
| ID | | |
| No. | sequence | Target |
| 7 | 5’ A^{&}sG^{&}sG^{&}sT*sGsCsTsCsAsGsGsAsCsTsCsC*sA&sT*sT*sT* 3’ | p38 alpha |
| 8 | 5’ A^{&}oG^{&}oG^{&}oT*oG&sCsTsCsAsGsGsAsCsTsCsC*oA^{&}oT*oT*oT* 3’ | p38 alpha |
| 9 | 5’C*sT*sC*sC*sA&sGsCsGsCsCsTsCsCsAsCsC*sA^{&}sG^{&}sG^{&}sC*3’ | TRADD |
| 10 | 5’C*oT*oC*oC*oA^{&}sGsCsGsCsCsTsCsCsAsCsC*oA^{&}oG^{&}oG^{&}oC*3’ | TRADD |
| 11 | 5’ C*sT*sG&s C*sT*sAs GsCsCs TsCsTs GsGsAs T*sT*sT*s G^{&}sA^{&}3’ | PTEN |
| 12 | 5’ C*oT*oG&o C*oT*sAs GsCsCs TsCsTs GsGsAs T*oT*oT*o G^{&}oA^{&} | PTEN |
| | 3’ | |
| 13 | 5’ CsCsGs GsTsAs CsCsCs C*sA^{&}sG^{&}s G&sT*sT*s C*sT*sT*s C*sA^{&} | m-Aaf |
| | 3’ | |
| 14 | 5’ CsCsGs GsTsAs CsCsCs C*oA^{&}oG^{&}o G^{&}oT*oT*o C*oT*oT*o | m-Aaf |
| | C*oA^{&} 3’ | |
| 15 | 5’ A^{&}sT*sA^{&}sG^{&}sT*sT*sT*sC*sA^{&}sC*sC*sT*sA^{&}sG^{&}sA^{&}sG^{&}s A^{&}s | PTEN |
| | A^{&}sA^{&}sG^{&} 3’ | |
| 16 | 5’ A^{&}oT*oA^{&}oG^{&}oT*oT*oT*oC*oA^{&}oC*oC*oT*oA^{&}oG^{&}o | PTEN |
| | A^{&}oG^{&}oA^{&}oA^{&}oA^{&}oG^{&} 3’ | |
| 17 | 5’ PEF TTT TTT TTT TTT TTT T*T*T*T* 3’ | Nuclease |
| | | Stability |

T* = 2’-O-[2-(methoxy)ethyl]-2-thio-5-methyluridine, C* = 2’-O-[2-(methoxy)ethyl]-2-thio-5-methylcytidine, T^{&} = 2’-O-[2-(methoxy)ethyl]-5-methyluridine, A^{&} = 2’-O-[2-(methoxy)ethyl]-adenosine, G^{&} = 2’-O-[2-(methoxy)ethyl[guanosine, C^{&} = 2’-O-]2-(methoxy)ethyl]-5-methylcytidine, o = P = O, s = P = S

What is claimed:

1. A compound of formula I: 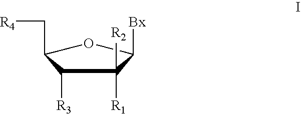
wherein:
R₁, R₂, and R₃ are selected such that:
R₃ is hydroxyl or protected hydroxyl, R₂ is H and R₁ is a sugar substituent group;
or R₃ is hydroxyl or protected hydroxyl, R₁ is H and R₂ is a sugar substituent group;
or R₂ is H, R₁ is hydroxyl or protected hydroxyl, and R₃ is a sugar substituent group;
R₄ is hydroxyl or protected hydroxy;
Bx has one of formulas II, III, IV, V, VI or VII: 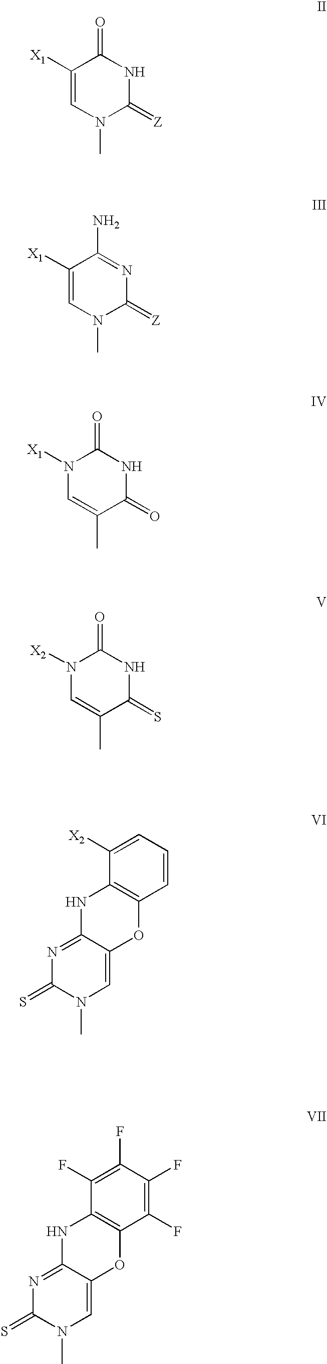
wherein:
X₁ is CH₂COOCH₃, CH₂COOCH₂CH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C—CH, CH₂C(═Y)NRᵤRᵥ, or CH₂NRᵤRᵥ;
X₂ is H, CH₃, CH₂COOCH₃, CH₂COOCH₂CH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C≡CH, CH₂C(═Y)NRᵤRᵥ, or CH₂NRᵤRᵥ;
Yis S, O, or NH;
Z is S or O;
n is an integer from 1 to 10;
each Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached; and
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
with the proviso that:
when Bx has formula II and Z is 0 then R₁ is not H, OH, OCH₃, OAc, protected hydroxyl or halogen; and
when Bx has formula II and Z is S then R₁ is not H, OH or protected hydroxyl;
when Bx has formula III, Z is O and X₁ is CH₂COOCH₃ then R₁ is not H;
when Bx has formula R₁, Z is O, X₁ is CH₂NH₂ then R₁ is not halogen; and
when Bx has formula III, Z is O and X₁ is CH₂C(═O)NRᵤRᵥ then at least one of Rᵤ and Rᵥ is not —(CH₂)₂NH₂.

2. The compound of claim 1 wherein R₂ is H and R₃ is OH.

3. The compound of claim 2 wherein Bx is a structure of formula II.

4. The compound of claim 3 wherein R₁ is F, OCH₂CH₂OCH₂CH₂N(CH₃)₂ or OCH₂CH₂OCH₃.

5. The compound of claim 4 wherein X₁ is CH₂N(CH₃)₂, CH₂C(═O)NH₂, CH₂N(COCF₃)CH₃, CH₂CO₂CH₃, CH₂CO₂CH₂CH₃, or CH₂NH(CH₃).

6. The compound of claim 5 wherein Z is O.

7. The compound of claim 5 wherein Z is S.

8. The compound of claim 2 wherein Bx is a structure of formula III.

9. The compound of claim 8 wherein R₁ is F or OCH₂CH₂OCH₃.

10. The compound of claim 9 wherein X₁ is CH₂N(CH₃)₂.

11. The compound of claim 10 wherein Z is S.

12. The compound of claim 2 wherein Bx is a structure of formula IV.

13. The compound of claim 12 wherein X₁ is CH₂N(CH₃)₂.

14. The compound of claim 2 wherein Bx is a structure of formula V.

15. The compound of claim 14 wherein X₂ is CH₂CO₂CH₃, CH₂CO₂CH₂CH₃, or CH₂CONH₂.

16. The compound of claim 2 wherein Bx is a structure of formula VII.

17. The compound of claim 1 wherein R₁ is H and R₃ is OH.

18. The compound of claim 17 wherein R₂ is F.

19. The compound of claim 18 wherein Bx is a structure of formula II.

20. The compound of claim 19 wherein Z is S.

21. The compound of claim 1 wherein R₂ is H and R₁ is OH.

22. The compound of claim 21 wherein Bx is a structure of formula II.

23. The compound of claim 22 wherein Z is S.

24. The compound of claim 23 wherein R₃ is OH.

25. The compound of claim 1 wherein said sugar substituent group is C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—Rₐ), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Ra), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol or a polyether of the formula (—O-alkyl)ₘₐ;
wherein each Rₐ is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;
or said sugar substituent group has one of formula Iₐ or IIₐ: 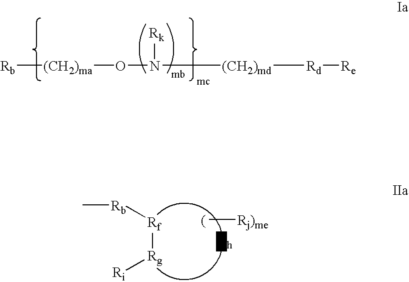
wherein:
R_{b} is O, S or NH;
R_{d} is a single bond, O, S or C(═O);
Rₑ is C₁-C₁₀ alkyl, N(Rₖ)(Rₘ), N(Rₖ)(R.ₙ, N═C(Rₚ)(R_{q}), N═C(Rₚ)(Rᵣ) or has formula IIIₐ; 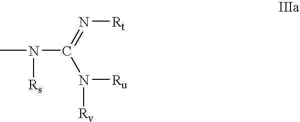
Rₚ and R_{q} are each independently hydrogen or C₁-C₁₀ alkyl;
Rᵣ is —Rₓ—R_{y};
each Rₛ, Rₜ, Rᵤ and Rᵥ is, independently, hydrogen, C(O)R_{w}, substituted or unsubstituted C₁-C₁₀alkyl, substituted or unsubstituted C₂-C₁₀alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₀ alkyl, substituted or unsubstituted C₂-C₁₀alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;
or Rₘ and Rₙ, together, are a nitrogen protecting group, are joined in a ring structure that optionally includes an additional heteroatom selected from N and O or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;
ma is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

26. The compound of claim 25 wherein said sugar substituent group is O(CH₂)₂OCH₃, O(CH₂)₂SCH₃, O(CH₂)₂ON(CH₃)₂, O(CH₂)₂O(CH₂)₂N(CH₃)₂, OCH₂C(═O)N(H)CH₃, OCH₃, O(CH₂)₂NH₂, O(CH₂)₂N(CH₃)₂, O(CH₂)₃NH₂, O(CH₂)₃N(H)CH₃, CH₂CH═CH₂, O(CH₂)₂S(O)CH₃, or fluoro.

27. An oligomeric compound comprising a plurality of nucleosides linked by internucleoside linking groups wherein at least one of said nucleosides is one of formulas VIII, IX or X: 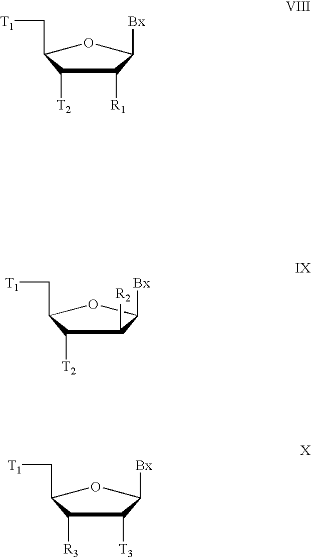
each T₁, T₂ and T₃ is, independently, hydroxyl, a protected hydroxyl or an internucleoside linking group covalently attaching a nucleoside, oligonucleoside, oligonucleotide or an oligomeric compound wherein at least one of T₁, T₂ and T₃ is an internucleoside linking group covalently attaching a nucleoside, oligonucleoside, oligonucleotide or an oligomeric compound;
each R₁, R₂ and R₃ is a sugar substituent group;
each Bx has one of formulas II, III, IV, V, VI or VII: 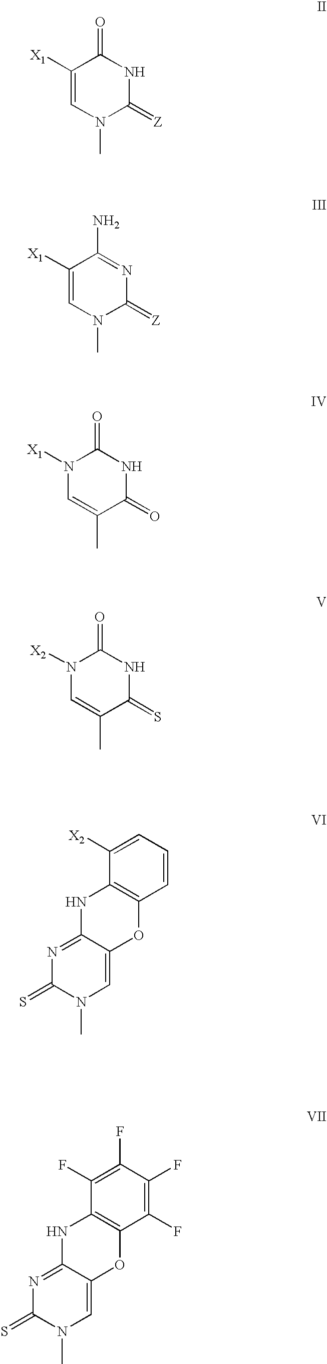
wherein:
X₁ is CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C═CH, CH₂C(═Y)NRᵤRᵥ, or CH₂NRᵤRᵥ;
X₂ is H, CH₃, CH₂COOCH₃, CH₂NHCH₂COOH, CH₂CH(OH)CH₂NRᵤRᵥ, CH₂NHCH₂C(═Y)NRᵤRᵥ, (CH₂))NHC(═Y)NRᵤRᵥ, CH₂C═CH, CH₂C(═Y)NRᵤRᵥ, or CH₂NRᵤRᵥ;
Y is S, O, or NH;
Zis S or O;
n is an integer from 1 to 10;
each Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached; and
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
with the proviso that:
when Bx is formula II and Z is O then R₁ is not H, OH, OCH₃, OAc, protected hydroxyl or halogen; and
when Bx is formula II and Z is S then R₁ is not H, OH or protected hydroxyl;
when Bx is formula III, Z is O and X₁ is CH₂COOCH₃ then R₁ is not H;
when Bx is formula III, Z is O, X₁ is CH₂NH₂ then R₁ is not halogen; and
when Bx is formula III, Z is O and X₁ is CH₂C(═O)NRᵤRᵥ then at least one of Rᵤ and Rᵥ is not —(CH₂)₂NH₂.

28. The oligomeric compound of claim 27 wherein essentially each of said internucleoside linking groups contains a phosphorus atom.

29. The oligomeric compound of claim 28 wherein each of said phosphorus containing internucleoside linking groups is, independently, selected from the group consisting of phosphodiester, phosphorothioate, chiral phosphorothioate, phosphorodithioate, phosphotriester, aminoalkylphosphotriester, methyl phosphonate, alkyl phosphonate, 5′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, selenophosphate and boranophosphate.

30. The oligomeric compound of claim 27 wherein essentially each of said internucleoside linking groups is a non-phosphorus containing internucleoside linking group.

31. The oligomeric compound of claim 30 wherein each of said non-phosphorus containing internucleoside linking groups is, independently, selected from the group consisting of morpholino, siloxane, sulfide, sulfoxide, sulfone, formacetyl, thioformacetyl, methylene formacetyl, thioformacetyl, sulfamate, methyleneimino, methylenehydrazino, sulfonate, sulfonamide, and amide.

32. The oligomeric compound of claim 31 wherein each of said non-phosphorus containing internucleoside linking groups is, independently, selected from the group consisting of CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂—.

33. The oligomeric compound of claim 27 comprising phosphorus and non-phosphorus containing internucleoside linking groups.

34. The oligomeric compound of claim 27 comprising a gapmer, hemimer or inverted gapmer.

35. The oligomeric compound of claim 27 comprising from about 8 to about 80 linked nucleosides.

36. The oligomeric compound of claim 27 comprising from about 8 to about 50 linked nucleosides.

37. The oligomeric compound of claim 27 comprising from about 12 to about 30 linked nucleosides.

38. The oligomeric compound of claim 27 wherein at least one of said monomeric subunits having a heterocyclic base moiety of formulas II, III, IV, V or VI comprises an arabinosy moiety.

39. The oligomeric compound of claim 27 wherein said sugar substituent group is C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—Rₐ), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH2), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol or a polyether of the formula (—O-alkyl)ₘₐ;
wherein each Rₐ is, independently, hydrogen, a protecting group or substituted or unsubstituted alkyl, alkenyl, or alkynyl wherein the substituent groups are selected from haloalkyl, alkenyl, alkoxy, thioalkoxy, haloalkoxy or aryl as well as halogen, hydroxyl, amino, azido, carboxy, cyano, nitro, mercapto, a sulfide group, a sulfonyl group and a sulfoxide group;
or said sugar substituent group has one of formula Iₐ or IIₐ: 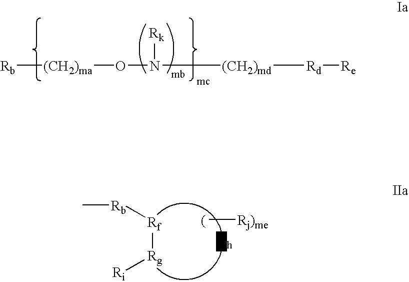
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ; 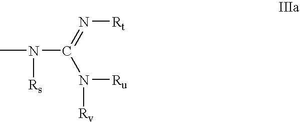
Rₚ and R_{q} are each independently hydrogen or C₁-C₁₀ alkyl;
Rᵣ is —Rₓ—R_{y};
each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester;
or Rₘ and Rₙ, together, are a nitrogen protecting group, are joined in a ring structure that optionally includes an additional heteroatom selected from N and O or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRk or CN;
ma is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

40. The oligomeric compound of claim 39 wherein said sugar substituent group is —O—CH₂CH₂OCH₃, —O(CH₂)₂ON(CH₃)₂, —O—(CH₂)₂—O—(CH₂)₂—N(CH₃)₂, —O—CH₃, —OCH₂CH₂CH₂NH₂, —CH₂—CH═CH₂ or fluoro.

41. An oligomeric compound having one of formulas XII or XIII: 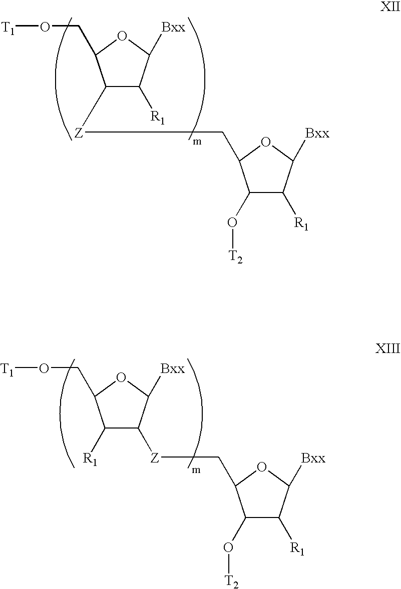
wherein:
each T₁ and T₂ is, independently, hydroxyl, a protected hydroxyl or an internucleoside linking group covalently attaching a nucleoside, oligonucleoside, oligonucleotide or an oligomeric compound;
each R₁ is a sugar substituent group;
m is from about 8 to about 80;
each Bxx is a heterocyclic base moiety having one of formulas II, III, IV, V, VI or VII: 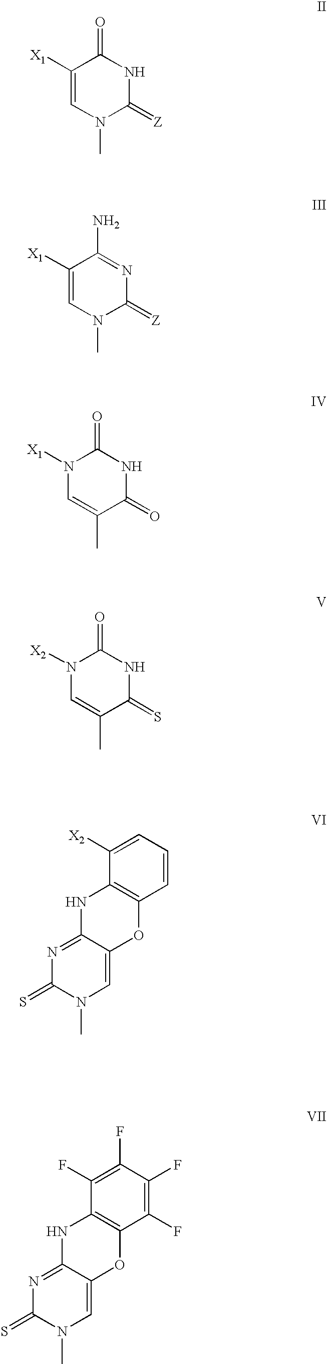
wherein:
X₁ is CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C—CH, CH₂C(═Y)NRᵤRᵥ, or CH₂NRₓRᵥv;
X₂ is H, CH₃, CH₂COOCH₃, CH₂NHCH₂COOH, CH₂CH(OH)CH₂NRᵤRᵥ, CH₂NHCH₂C(═Y)NRᵤRᵥ, (CH₂)ₙNHC(═Y)NRᵤRᵥ, CH₂C—CH, CH₂C(═Y)NRᵤRᵥ, or CH₂NRₓRᵥ;
Y is S, O, or NH;
Z is S or O;
n is an integer from 1 to about 10;
each Rᵤ and Rᵥ, is, independently, hydrogen, C(O)R_{w}, substituted or unsubstituted C₁-C₁₀ alkyl, substituted or unsubstituted C₂-C₁₀ alkenyl, substituted or unsubstituted C₂C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached; and
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
with the proviso that:
when Bx is formula II and Z is O then R₁ is not H, OH, OCH₃, OAc, protected hydroxyl or halogen; and
when Bx is formula II and Z is S then R₁ is not H, OH or protected hydroxyl;
when Bx is formula III, Z is O and X₁ is CH₂COOCH₃ then R₁ is not H;
when Bx is formula III, Z is O, X₁ is CH₂NH₂ then R₁ is not halogen; and
when Bx is formula III, Z is O and X₁ is CH₂C(═O)NRᵤRᵥ then at least one of Rᵤ and Rᵥ is not —(CH₂)₂NH₂.

42. The oligomeric compound of claim 41 wherein said internucleoside linking group is a phosphorus-containing internucleoside linking group.

43. The oligomeric compound of claim 42 wherein said internucleoside linking group is selected from the group consisting of phosphorothioate, chiral phosphorothioate, phosphorodithioate, phosphotriester, aminoalkylphosphotriester, methyl phosphonate, alkyl phosphonate, 5′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, selenophosphate and boranophosphate.

44. The oligomeric compound of claim 43 wherein said internucleoside linking group is an amide, methylene (methylimino) or a formacetal.

45. The oligomeric compound of claim 41 wherein said heterocyclic base moieties in addition to said at least one having one of formulas II, III, IV, V, VI or VII are, independently, selected from the group consisting of adeninyl, guaninyl, thyminyl, cytosinyl, uracilyl, 5-methylcytosinyl (5-me-C), 5-hydroxymethyl cytosinyl, xanthinyl, hypoxanthinyl, 2-aminoadeninyl, alkyl derivatives of adeninyl and guaninyl, 2-thiouracilyl, 2-thiothyminyl, 2-thiocytosinyl, 5-halouracilyl, 5-halocytosinyl, 5-propynyl uracilyl, 5-propynyl cytosinyl, 6-azo uracilyl, 6-azo cytosinyl, 6-azo thyminyl, 5-uracilyl (pseudouracil), 4-thiouracilyl, 8-substituted adeninyls and guaninyls, 5-substituted uracilyls and cytosinyls, 7-methylguaninyl, 7-methyladeninyl, 8-azaguaninyl, 8-azaadeninyl, 7-deazaguaninyl, 7-deazaadeninyl, 3-deazaguaninyl and 3-deazaadeninyl.

46. The oligomeric compound of claim 41 wherein m is from about 8 to about 50.

47. The oligomeric compound of claim 41 wherein m is from about 12 to about 30.

48. A pharmaceutical composition comprising at least one oligomeric compound of claim 41.
